SEQUENCE LISTING (75 sequences)

SEQ ID NO: 1 - protein, 11 aa, synthetic construct
QTSQGVGSDLH

SEQ ID NO: 2 - protein, 7 aa, synthetic construct
HTSSVED

SEQ ID NO: 3 - protein, 5 aa, synthetic construct
QVLQF

SEQ ID NO: 4 - protein, 5 aa, synthetic construct
AHILF

SEQ ID NO: 5 - protein, 17 aa, synthetic construct
WIKPQYGAVNFGGGFRD

SEQ ID NO: 6 - protein, 13 aa, synthetic construct
DRSYGDSSWALDA

SEQ ID NO: 7 - protein, 103 aa, synthetic construct [sequence omitted]

SEQ ID NO: 8 - protein, 122 aa, synthetic construct [sequence omitted]

SEQ ID NO: 9 - protein, 241 aa, synthetic construct
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGSGGGSGGGSGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIK

SEQ ID NO: 10 - protein, 649 aa, synthetic construct
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGSGGGSGGGSGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 16 aa, synthetic construct
GGGSGGGSGGGSGGGS

SEQ ID NO: 12 - protein, 17 aa, synthetic construct
SSGGGGSGGGGSGGGGS

SEQ ID NO: 13 - protein, 4 aa, synthetic construct
GGGS

SEQ ID NO: 14 - protein, 5 aa, synthetic construct
GGGGS

SEQ ID NO: 15 - protein, 21 aa, Homo sapiens
LCYLLDGILFIYGVILTALFL

SEQ ID NO: 16 - protein, 27 aa, Homo sapiens
FWVLVVVGGVLACYSLLVTVAFIIFWV

SEQ ID NO: 17 - protein, 28 aa, Homo sapiens
MFWVLVVVGGVLACYSLLVTVAFIIFWV

SEQ ID NO: 18 - protein, 22 aa, Homo sapiens
MALIVLGGVAGLLLFIGLGIFF

SEQ ID NO: 19 - protein, 21 aa, Homo sapiens
IYIWAPLAGTCGVLLLSLVIT

SEQ ID NO: 20 - protein, 23 aa, Homo sapiens
IYIWAPLAGTCGVLLLSLVITLY

SEQ ID NO: 21 - protein, 24 aa, Homo sapiens
IYIWAPLAGTCGVLLLSLVITLYC

SEQ ID NO: 22 - protein, 27 aa, Homo sapiens
IISFFLALTSTALLFLLFFLTLRFSVV

SEQ ID NO: 23 - protein, 21 aa, Homo sapiens
PFFFCCFIAVAMGIRFIIMVA

SEQ ID NO: 24 - protein, 10 aa, synthetic construct
GGGSSGGGSG

SEQ ID NO: 25 - protein, 12 aa, synthetic construct
ESKYGPPCPPCP

SEQ ID NO: 26 - protein, 12 aa, synthetic construct
ESKYGPPCPSCP

SEQ ID NO: 27 - protein, 22 aa, synthetic construct
ESKYGPPCPPCPGGGSSGGGSG

SEQ ID NO: 28 - protein, 39 aa, synthetic construct
IEVMYPPPYLDNEKSNGTIIHVKGKHLCPSPLFPGPSKP

SEQ ID NO: 29 - protein, 48 aa, synthetic construct
AKPTTTPAPRPPTPAPTIASQPLSLRPEACRPAAGGAVHTRGLDFACD

SEQ ID NO: 30 - protein, 45 aa, synthetic construct
TTTPAPRPPTPAPTIASQPLSLRPEACRPAAGGAVHTRGLDFACD

SEQ ID NO: 31 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 229 aa, synthetic construct
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 41 aa, Homo sapiens
RSKRSRLLHSDYMNMTPRRPGPTRKHYQPYAPPRDFAAYRS

SEQ ID NO: 37 - protein, 41 aa, Homo sapiens
RSKRSRGGHSDYMNMTPRRPGPTRKHYQPYAPPRDFAAYRS

SEQ ID NO: 38 - protein, 42 aa, Homo sapiens
KRGRKKLLYIFKQPFMRPVQTTQEEDGCSCRFPEEEEGGCEL

SEQ ID NO: 39 - protein, 42 aa, Homo sapiens
ALYLLRRDQRLPPDAHKPPGGGSFRTPIQEEQADAHSTLAKI

SEQ ID NO: 40 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 41-43 - skipped (3 entries)

SEQ ID NO: 44 - DNA, 10008 bp, synthetic construct
  source 1..10008: mol_type = other DNA
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgctcctgctcgtgacatccctcctgttgtgcgaacttccacaccccgccttcctgctgatccctcgcgcccacttggtgcagtcgggtactgcgatgaagaagcccggagcatccgtgagagtgtcgtgccaaaccagcggatacacctttaccgctcacatcctgttctggttccgccaagccccgggccggggtctggaatgggtcggatggatcaagccgcagtacggcgcagtgaatttcggcgggggattccgggatcgggtcactctgacccgcgatgtgtacagagagatcgcctatatggacatcaggggactgaagcctgatgacaccgctgtgtactactgcgcccgggaccggtcgtacggggactcctcatgggccctggatgcctggggacagggcaccactgtggtggtgtccgcgggggggggatccgggggtggcagcgggggcggctccggtggtggaagctacattcacgtcacccagtccccttcgtcactttccgtgagcattggggatcgcgtgaccatcaactgtcagacgtcacagggcgtgggctcggacctccattggtaccagcataagccgggaagggccccgaagctcctgatccatcacacctcatccgtggaggacggagtcccctcccggttctccggctcgggattccacacttccttcaacctgaccattagcgacctccaagcggacgacattgccacttactactgccaagtgctgcagttcttcggacggggcagcagactgcacatcaaggaatccaaatatggaccgccctgcccaccctgtccggccccggaattcgagggtggcccgtctgtgttcctgtttccacccaaacctaaggacactctgatgatcagccgcacccctgaggtaacctgcgtggtggtggacgtgtcccaggaagatcccgaggtccagttcaattggtacgtggacggcgtggaagtgcacaatgccaagaccaagcccagagaggaacagttccaaagcacctaccgggtggtgtccgtgctgaccgtgctgcaccaggactggctgaacggcaaagagtacaagtgcaaggtgtccaacaagggcctgcccagcagcatcgagaaaaccatcagcaaggccaagggccagccccgcgagccccaggtgtacacactgccccccagccaggaagagatgaccaagaaccaggtgtccctgacctgcctggtcaagggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtctctgagcctgagcctgggcaagatggccctgatcgtgctgggcggagtggccggactgctgctgtttatcggcctgggcatcttcttcaagcggggcagaaagaagctgctgtacatcttcaagcagcccttcatgcggcccgtgcagaccacccaggaagaggacggctgcagctgccggttccccgaggaagaggaaggcggctgcgagctgggaggcggcagagtgaagttcagccggtccgccgacgcccctgcctaccagcagggccagaaccagctgtacaacgagctgaacctgggcaggcgggaggaatacgacgtgctggacaagcggagaggccgggaccctgagatgggcggcaagcccaggcggaagaaccctcaggaaggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctaggggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 45 - protein, 24 aa, synthetic construct
LEGGGEGRGSLLTCGDVEENPGPR

SEQ ID NO: 46 - protein, 354 aa, Homo sapiens
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 47 - protein, 323 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 48-49 - skipped (2 entries)

SEQ ID NO: 50 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 51 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTYDALHMQALPPR

SEQ ID NO: 52 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLFQGLSTATKDTFDALHMQALPPR

SEQ ID NO: 53 - protein, 112 aa, synthetic construct
RVKFSRSADAPAYQQGQNQLYNELNLGRREEYDVLDKRRGRDPEMGGKPRRKNPQEGLYNELQKDKMAEAFSEIGMKGERRRGKGHDGLFQGLSTATKDTFDALHMQALPPR

SEQ ID NO: 54 - protein, 112 aa, synthetic construct
RVKFSRSADAPAYQQGQNQLYNELNLGRREEYDVLDKRRGRDPEMGGKPRRKNPQEGLFNELQKDKMAEAYSEIGMKGERRRGKGHDGLFQGLSTATKDTFDALHMQALPPR

SEQ ID NO: 55 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTFDALHMQALPPR

SEQ ID NO: 56 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTYDALHMQALPPR

SEQ ID NO: 57 - protein, 561 aa, Human betaherpesvirus 5
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 58 - protein, 902 aa, synthetic construct
MVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 59 - protein, 407 aa, Human betaherpesvirus 5
MVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQ

SEQ ID NO: 60 - skipped

SEQ ID NO: 61 - protein, 496 aa, Human betaherpesvirus 5
MGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 62 - protein, 496 aa, synthetic construct
MGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEATMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 63 - protein, 496 aa, synthetic construct
MGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTAPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 64 - protein, 496 aa, synthetic construct
MGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEATMPVTAPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 65 - protein, 22 aa, synthetic construct
MLLLVTSLLLCELPHPAFLLIP

SEQ ID NO: 66 - protein, 15 aa, synthetic construct
GGGGSGGGGSGGGGS

SEQ ID NO: 67 - DNA, 22 bp, synthetic construct
  source 1..22: mol_type = other DNA
gcctcaataaagcttgccttga

SEQ ID NO: 68 - DNA, 21 bp, synthetic construct
  source 1..21: mol_type = other DNA
ggcgccactgctagagatttt

SEQ ID NO: 69 - DNA, 30 bp, synthetic construct
  source 1..30: mol_type = other DNA
  modified_base 1: mod_base = OTHER; note = 5' Fluorescein
  modified_base 30: mod_base = OTHER; note = 3' Iowa Black FQ quencher
aagtagtgtgtgcccgtctgttgtgtgact

SEQ ID NO: 70 - DNA, 20 bp, synthetic construct
  source 1..20: mol_type = other DNA
gactgtagtccaggaatatg

SEQ ID NO: 71 - DNA, 18 bp, synthetic construct
  source 1..18: mol_type = other DNA
tgtttcctgccctgtctc

SEQ ID NO: 72 - DNA, 24 bp, synthetic construct
  source 1..24: mol_type = other DNA
  modified_base 1: mod_base = OTHER; note = 5' cyanine 5
  modified_base 24: mod_base = OTHER; note = 3' Iowa Black FQ quencher
cttggtagcagttcatgtagccag

SEQ ID NO: 73 - protein, 121 aa, synthetic construct
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVS

SEQ ID NO: 74 - protein, 335 aa, Homo sapiens
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 75 - protein, 5 aa, synthetic construct
  VARIANT 1..5: note = This sequence may encompass 1-5 residues
GGGGG

(19) United States
(12) Patent Application Publication
(10) Pub. No.: US 2025/0249102 A1
(43) Pub. Date: Aug. 7, 2025

(54) CMV-HIV SPECIFIC CHIMERIC ANTIGEN RECEPTOR T CELLS

(71) Applicant: City of Hope, Duarte, CA (US)

(72) Inventors: Xiuli Wang, Temple City, CA (US); John Zaia, Duarte, CA (US); Lihua Elizabeth Budde, Duarte, CA (US); Stephen J. Forman, Duarte, CA (US); Don J. Diamond, Duarte, CA (US)

(21) Appl. No.: 18/856,156

(22) PCT Filed: Apr. 13, 2023
(86) PCT No.: PCT/US2023/065749

§ 371 Date: Oct. 11, 2024

Related U.S. Application Data
(60) Provisional application No. 63/330,726, filed on Apr. 13, 2022.

(51) Int. Cl. A61K 40/46; A61K 40/11; A61K 40/31; C07K 14/725; C07K 16/08; C07K 16/10

(52) U.S. Cl. CPC A61K 40/46; A61K 40/11; A61K 40/31; C07K 14/7051; C07K 16/089; C07K 16/1045; C07K 2317/33; C07K 2317/622

(57) ABSTRACT

This disclosure relates, inter alia, to compositions comprising and methods making and using T cells expressing both a chimeric antigen receptor (CAR) targeted to HIV and a T cell receptor targeted to cytomegalovirus (CMV).

### CLAIM OF PRIORITY

This application claims the benefit of U.S. Provisional Application Ser. No. 63/330,726, filed on Apr. 13, 2022.

### TECHNICAL FIELD

This disclosure relates to T cells expressing both a chimeric antigen receptor (CAR) targeted to HIV and a T cell receptor targeted to cytomegalovirus (CMV).

### BACKGROUND

Combination antiretroviral therapy (ART) achieves undetectable plasma viremia¹ but fails to cure HIV infection due to the persistence of a latent virus reservoir containing replication-competent HIV-1.² Alternative cellular immune strategies of adoptive immunotherapy have been proposed using either expansion of endogenous HIV-specific T cells or autologous T cells or natural killer (NK) redirected to HIV-infected cells.³⁻⁵ The first-generation of HIV-CAR T cells was developed almost 25 years ago by engineering the extracellular domain of the CD4 receptor on the surface of T cells.^{6, 7} These CD4-based CAR T cells were tested in three clinical trials in HIV-seropositive individuals^{4, 8-10}, but this strategy was aborted due to negligible clinical efficacy. Several factors may explain the lack or minimal antiviral efficacy, such as limited CAR activity in the absence of an intracellular co-stimulatory signaling domain, low expansion and minimal persistence of CAR T cells due to insufficient exposure to HIV antigens because CAR-recipients remained on ART, or the susceptibility of CD4-CAR-expressing T cells to HIV infection. Of note, a low level of these first-generation CD4-CAR T cells was detected a decade later in these recipients, suggesting that long-term persistence is possible.¹¹ Since then, CARs have been optimized by adding the co-stimulatory domains CD28 or 4-1BB, and in the setting of B-cell malignancies, have shown improved efficacy and persistence with promising therapeutic results.¹²⁻¹⁵ In addition, a series of broadly neutralizing antibodies (bNAbs) directed to the HIV-1 envelope glycoprotein gp120 were identified in HIV-infected non-progressors and used to develop bNAbs-derived CAR T cells that efficiently kill gp120-expressing cells in vitro.^{16, 17} To limit the emergence of resistance to HIV, these bNAb-based CAR T cells should be effective against nearly all strains of HIV. Notably, Huang et al.¹⁸ isolated a bNAb named N6 that potently neutralizes 98% of HIV-1 isolates including 16 of 20 that evolved to circumvent common mechanisms of resistance.

It may require high CAR T cell number to achieve therapeutic benefit, as currently tested in a CD4-based CAR T cell clinical trial [NCT03617198]. Another approach to enhance CAR T cell persistence involves the engagement of viral antigens as stimulatory factors for CAR T cells.¹⁹⁻²³

### SUMMARY

The present disclosure is based, at least in part, on the discovery that T cells expressing a chimeric antigen receptor (CAR) specific for HIV and a T cell receptor specific for cytomegalovirus (CMV) (“CMV-HIV T cells”), in some cases, together with a CMV vaccine (e.g., one or more CMV antigens or a nucleic acid encoding one or more CMV antigens) can be used to treat subjects infected with HIV. Some of the main issues facing HIV patients still today is CAR T cell activation and maintaining persistence in vivo. HIV patients taking antiretroviral drugs face these issues and so far, there's no data showing methods effective ways to expand and maintain CAR T cells in such a patient, as the cells disappear functionally. Furthermore, isolating and expanding a population of CMV specific T cells presents additional difficulties for numerous reasons. For example, this specific T cell population is typically very low in subjects, so manufacturing enough CMV CAR T cells to administer to a subject can be very difficult. In the present application, the compositions and methods described herein result in increased expansion and persistence of CAR T cells, which include a CMV-HIV T cell.

Described herein, inter alia, are nucleic acid molecules encoding a chimeric antigen receptor, wherein the chimeric antigen receptor comprises: an scFv that binds HIV Env; a spacer domain; a transmembrane domain; a costimulatory domain; and a CD3ζ signaling domain.

In various embodiments: the scFv comprises: a VL domain comprising: a light chain CDR1 comprising QTSQGVGSDLH (SEQ ID NO:1), a light chain CDR2 comprising HTSSVED (SEQ ID NO:2), a light chain CDR3 comprising QVLQF (SEQ ID NO:3); and a VH domain comprising: a heavy chain CDR1 comprising AHILF (SEQ ID NO:4), a heavy chain CDR2 comprising WIKPQYGAVNFGGGFRD (SEQ ID NO:5), and a heavy chain CDR3 comprising DRSYGDSSWALDA (SEQ ID NO:6); the scFv comprises: (a) a light chain variable domain that is at least 90%, 95% or 98% identical to: YIHVTQSPSSLSVSIGDRVTINCQTSQGVGSDLHWYQHKPGRAPKLLIHHTSSVEDGVPSR FSGSGFHTSFNLTISDLQADDIATYYCQVLQFFGRGSRLHIK (SEQ ID NO:7); and (b) a heavy chain variable domain that is at least 90%, 95% or 98% identical to: RAHLVQSGTAMKKPGASVRVSCQTSGYTFTAHILFWFRQAPGRGLEWVGWIKPQYGAVN FGGGFRDRVTLTRDVYREIAYMDIRGLKPDDTAVYYCARDRSYGDSSWALDAWGQGTTVV VSA (SEQ ID NO:8); the scFV comprises: a light chain variable domain comprising YIHVTQSPSSLSVSIGDRVTINCQTSQGVGSDLHWYQHKPGRAPKLLIHHTSSVEDGVPSR FSGSGFHTSFNLTISDLQADDIATYYCQVLQFFGRGSRLHIK (SEQ ID NO:7); and a heavy chain variable domain comprising RAHLVQSGTAMKKPGASVRVSCQTSGYTFTAHILFWFRQAPGRGLEWVGWIKPQYGAVN FGGGFRDRVTLTRDVYREIAYMDIRGLKPDDTAVYYCARDRSYGDSSWALDAWGQGTTVV VSA (SEQ ID NO: 9); and the scFv comprises a VL domain that is 95% identical to YIHVTQSPSSLSVSIGDRVTINCQTSQGVGSDLHWYQHKPGRAPKLLIHHTSSVEDGVPSR FSGSGFHTSFNLTISDLQADDIATYYCQVLQFFGRGSRLHIK (SEQ ID NO:7) and includes the following CDR sequences: QTSQGVGSDLH (VL-CDR1; SEQ ID NO:1), HTSSVED (VL-CDR2; SEQ ID NO:2), and QVLQF (VL-CDR3; SEQ ID NO:3); and a VH domain that is 95% identical to RAHLVQSGTAMKKPGASVRVSCQTSGYTFTAHILFWFRQAPGRGLEWVGWIKPQYGAVN FGGGFRDRVTLTRDVYREIAYMDIRGLKPDDTAVYYCARDRSYGDSSWALDAWGQGTTVV VSA (SEQ ID NO:8) and includes the following CDR sequences AHILF (VH-CDR1; SEQ ID NO:4) WIKPQYGAVNFGGGFRD (VH-CDR2; SEQ ID NO:5), and DRSYGDSSWALDA (VH-CDR3; SEQ ID NO:6).

Described herein are nucleic acid molecules encoding a chimeric antigen receptor, wherein the chimeric antigen receptor comprises: a scFv comprising or consisting of RAHLVQSGTAMKKPGASVRVSCQTSGYTFTAHILFWFRQAPGRGLEWVGWIKPQYGAVN FGGGFRDRVTLTRDVYREIAYMDIRGLKPDDTAVYYCARDRSYGDSSWALDAWGQGTTVV VSAGGGSGGGSGGGSGGGSYIHVTQSPSSLSVSIGDRVTINCQTSQGVGSDLHWYQHKP GRAPKLLIHHTSSVEDGVPSRFSGSGFHTSFNLTISDLQADDIATYYCQVLQFFGRGSRLHI K (SEQ ID NO:9); a spacer comprising a sequence selected from the group consisting of: SEQ ID NOs: 24-34; a transmembrane domain comprising a sequence selected from the group consisting of SEQ ID NOs: 15-23; a costimulatory domain comprising a sequence selected from the group consisting of SEQ ID NOs: 36-40, and a CD3ζ signaling domain comprising SEQ ID NO: 35.

In the case of any of the nucleic acid molecules: the spacer region comprises an amino acid sequence selected from the group consisting of SEQ ID NOs: 24-34 or a variant thereof having 1, 2, 3, 4, or 5 amino acid substitutions; the transmembrane domain selected from the group consisting of: a CD4 transmembrane domain, a CD8 transmembrane domain, a CD28 transmembrane domain, and a CD3ζ transmembrane domain; the costimulatory domain selected from the group consisting of: a 28 costimulatory domain, a 41-BB costimulatory domain, an OX40 costimulatory domain, and a 2B4 costimulatory domain; the chimeric antigen receptor comprises the amino acid sequence

(SEQ ID NO: 10)
RAHLVQSGTAMKKPGASVRVSCQTSGYTFTAHILFWFRQAPGRGLEWVG
WIKPQYGAVNFGGGFRDRVTLTRDVYREIAYMDIRGLKPDDTAVYYCAR
DRSYGDSSWALDAWGQGTTVVVSAGGGSGGGSGGGSGGGSYIHVTQSPS
SLSVSIGDRVTINCQTSQGVGSDLHWYQHKPGRAPKLLIHHTSSVEDGV
PSRFSGSGFHTSFNLTISDLQADDIATYYCQVLQFFGRGSRLHIKESKY
GPPCPPCPAPEFEGGPSVFLFPPKPKDTLMISRTPEVTCVVVDVSQEDP
EVQFNWYVDGVEVHNAKTKPREEQFQSTYRVVSVLTVLHQDWLNGKEYK
CKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQ
EGNVFSCSVMHEALHNHYTQKSLSLSLGKMALIVLGGVAGLLLFIGLGI
FFKRGRKKLLYIFKQPFMRPVQTTQEEDGCSCRFPEEEEGGCELGGGRV
KFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDT
YDALHMQALPPR

with 0, 1, 2, 3, 4 of 5 single amino acid substitutions; in some embodiments, the amino acid substitutions are conservative; in some embodiments, the amino acid substitutions are not in CDRs; in some embodiments, the nucleic acid molecule further comprises an interdomain linker consisting of 1-5 amino acids between one or more of: the scFV and the spacer domain, the spacer domain and the transmembrane domain, the transmembrane domain and the co-stimulatory domain, and/or the costimulatory domain and the CD3ζ signaling domain; the interdomain linker can consist of 1-5 glycine; in some embodiments, the CAR further comprises an interdomain linker consisting of the sequence GGG is located between the costimulatory domain and the CD3ζ signaling domain.

In some embodiments, a CAR comprises: a scFv comprising or consisting of SEQ ID NO:9 or a variant thereof having 1, 2, 3, 4, or 5, amino acid substitutions that are not in a CDR; a spacer comprising a sequence selected from the group consisting of: SEQ ID NOs: 24-34 or a variant thereof having 1, 2, 3, 4, or 5, amino acid substitutions; a transmembrane domain comprising a sequence selected from the group consisting of SEQ ID NOs: 15-23 or a variant thereof having 1, 2, 3, 4, or 5, amino acid substitutions; a costimulatory domain comprising a sequence selected from the group consisting of SEQ ID NOs: 36-40 or a variant thereof having 1, 2, 3, 4, or 5, amino acid substitutions; and a CD3ζ signaling domain comprising SEQ ID NO: 35 or a variant thereof having 1, 2, 3, 4, or 5, amino acid substitutions. In some embodiments, the amino acid substitutions are conservative.

Also described herein are immune cells harboring any of the nucleic acid molecules described herein. Also described herein are immune cells expressing any of the CAR described herein.

In various embodiments: the immune cell is a T cell expressing a T cell receptor specific for CMV (CMV specific T cell).

Also described are populations of cells comprising CMV specific T cells harboring any of the nucleic acid molecules described herein. Also described are populations of CMV-specific T cells expressing any of the HIV CAR described herein.

In various embodiments: at least 20%, 30%, 40%, or 50% of the CMV specific T cells are CD8+ T cells.

Also described are methods of preparing the population of CMV specific T cells expressing an HIV CAR, the method comprising: isolating a cell population comprising PBMC from a blood sample obtained from a CMVP^{pos} subject; contacting the cell population with a CMV antigen to stimulate CMV specific T cells; isolating a sub-population CMV specific T cells (e.g, by selecting IFNγ-secreting T cells from the cell population); and transducing cells in the sub-population of IFNγ-secreting T cells with a vector comprising any of the nucleic acid molecules described herein.

In some embodiments, methods of preparing T cells specific for cytomegalovirus (CMV) and expressing a chimeric antigen receptor (CAR) are described herein. In some embodiments, the method comprises: (a) providing a cell population comprising T cells (e.g., PBMC) from a cytomegalovirus CMV seropositive human donor; (b) exposing the cell population (e.g, PBMC) to at least one CMV antigen; (c) treating the exposed cells to produce a population of cells enriched for stimulated cells specific for CMV; (d) transducing at least a portion of the enriched population of cells with a vector expressing a CAR, thereby preparing T cells specific for CMV and expressing a CAR. In various cases: the step of treating the exposed cells (e.g., using a selection step) to produce a population of cells enriched for stimulated cells specific for CMV comprises treating the stimulated cells to produce a population of cells enriched for cells expressing an activation marker (e.g., IFN-γ or IL-13); the PBMC are cultured for less than 5 days (less than 4, 3, 2, 1 days) prior to exposure to the CMV antigen; the cells are exposed to the CMV antigen for fewer than 3 days (fewer than 48 hrs, 36 hrs, 24 hrs) the CMV antigen is pp65 protein or an antigenic portion thereof, the CMV antigen comprises two or more different antigenic CMV pp65 peptides; the step of transducing the enriched population of cells does not comprise CD3 stimulation; the step of transducing the enriched population of cells does not comprise CD28 stimulation; the step of transducing the enriched population of cells does not comprise CD3 stimulation or CD28 stimulation; the enriched population of cells is at least 40% (e.g., 50%, 60%, 70%) IFN-γ positive, at least 20% (e.g., 25%, 30%, 35%) CD8 positive, and at least 20% (e.g., 25%, 30%, 35%) CD4 positive; the enriched population of cells are cultured for fewer than 10 (fewer than 9, 8, 7, 5, 3, 2) days prior to the step of transducing the enriched population of cells with a vector encoding a CAR. In some cases, the T cells are from a CMV positive donor and are exposed to a CMV antigen such as CMV pp65 or a mixture of CMV protein peptides (for example 10-20 amino acid peptides that are fragments of pp65) in the presence of IL-2 to create a population of stimulated cells. In some cases, the population of stimulated cells is treated to prepare a population of cells that express IFN-γ.

In various embodiments: a sub-population of CMV T cells (e.g., IFNγ-secreting T cells expressing a T cell receptor specific for CMV) are cultured in the presence of one or both of exogenous IL-2 and exogenous IL-15 before transduction, after transduction or both before and after transduction; IL-2 is added to at 50 U/mL and IL-15 is added to 1 ng/mL; the sub-population of IFNγ-secreting T cells are cultured in the presence of at least one anti-retroviral drug before transduction, after transduction or both before and after transduction; the at least one anti-retroviral drug is selected from the group consisting of: 1) a HIV protease inhibitor (e.g., tipranavir, atazanavir, indinavir, darunavir or fosamprenavir); and 2) a HIV fusion inhibitor, an HIV entry inhibitor, HIV attachment inhibitor, HIV post-attachment inhibitor (e.g., maraviroc, Ibalizuma-buiyk, fostemsavir) and the cells are not cultured in the presence of a reverse transcriptase inhibitor; the blood sample is from a subject infected with HIV (e.g., a subject that has been previously been administered one or more anti-retroviral drugs. Preferably they are cultured in the presence of darunavir and enfuvirtide and in the absence of a reverse transcriptase inhibitor and/or an anti-retroviral drug that interferes with lentiviral replication or the replication of the viral vector carrying the sequence encoding the HIV-targeted chimeric antigen receptor.

Also described are methods for treating a subject infected with HIV, the method comprising administering: (a) CMV-specific T cells expressing chimeric antigen receptor comprising: an scFv that binds HIV Env; a spacer domain; a transmembrane domain; a costimulatory domain; and a CD3ζ signaling domain; and, optionally, (b) at least one CMV antigen or a nucleic acid molecule encoding at least one CMV antigen. In some embodiments, the at least one CMV antigen or a nucleic acid molecule encoding at least one CMV antigen is administered at the same time that of the CMV-HIV CAR T cells are administered. In some embodiments, the at least one CMV antigen or a nucleic acid molecule encoding at least one CMV antigen is administered before the administration of the CMV-HIV CAR T cells. In some embodiments, the at least one CMV antigen or a nucleic acid molecule encoding at least one CMV antigen is administered after the administration of the CMV-HIV CAR T cells. In some embodiments, the at least one CMV antigen or a nucleic acid molecule encoding at least one CMV antigen is administered before and after the administration of the CMV-HIV CAR T cells. In some embodiments, the at least one CMV antigen or a nucleic acid molecule encoding at least one CMV antigen is administered in single or repeat dosing. In some embodiments, the CMV-HIV CAR T cells are administered in single or repeat dosing.

Useful doses of CMV HIV T cells include about 5×10⁸, 10×10⁶, 15×10⁶, 20×10⁶, 25×10⁶, 30×10⁶, 35×10⁶, 40×10⁶, 45×10⁶, 50×10⁶, 55×10⁶, 60×10⁶, 65×10⁶, 70×10⁶, 75×10⁸, 80×10⁸, 85×10⁵, 90×10⁸, 95×10⁸, and 100×10⁸ cells. In some embodiments, a single dose of CMV-HIV CAR T cells is administered to the patient. In some embodiments, a second dose of CMV-HIV CAR T cells is administered to the patient. Suitable modes of administration include injection, infusion, instillation, or ingestion. Injection includes, without limitation, intravenous, intramuscular, intra-arterial, intrathecal, intraventricular, intradermal, intraperitoneal, subcutaneous injection, and infusion. In some embodiments, a subject is administered a population of CMV HIV T cells in a single intravenous (IV) infusion.

In some embodiments, the subject is administered the at least one CMV antigen or a nucleic acid molecule encoding at least one CMV antigen prior to the administration of the CMV-HIV CAR T cells. In some embodiments, the at least one CMV antigen or a nucleic acid molecule encoding at least one CMV antigen is administered one, two, three, four, five, six, seven, eight, nine, or ten days before the administration of the CMV-HIV CAR T cells. In some embodiments, the at least one CMV antigen or a nucleic acid molecule encoding at least one CMV antigen is administered about 1, 2, 3, 4, 5, 6, 7, 8, 9, 10, 11, 12, 14, 16, 18, 20, 21, 22, 23, 24, 25, 26, 28, 36, 48, 60, 75, 90, 120, 150, 180, 210, 240, 270, 300, 330, and/or 360 hours before the administration of the CMV-HIV CAR T cells. In some embodiments, the at least one CMV antigen or a nucleic acid molecule encoding at least one CMV antigen is administered about one, two, three, or four weeks before the administration of the CMV-HIV CAR T cells.

In some embodiments, the subject is administered the at least one CMV antigen or a nucleic acid molecule encoding at least one CMV antigen following the administration of the CMV-HIV CAR T cells. In some embodiments, the at least one CMV antigen or a nucleic acid molecule encoding at least one CMV antigen is administered one, two, three, four, five, six, seven, eight, nine, or ten days after the administration of the CMV-HIV CAR T cells. In some embodiments, the at least one CMV antigen or a nucleic acid molecule encoding at least one CMV antigen is administered about 1, 2, 3, 4, 5, 6, 7, 8, 9, 10, 11, 12, 14, 16, 18, 20, 21, 22, 23, 24, 25, 26, 28, 36, 48, 60, 75, 90, 120, 150, 180, 210, 240, 270, 300, 330, and/or 360 hours after the administration of the CMV-HIV CAR T cells. In some embodiments, the at least one CMV antigen or a nucleic acid molecule encoding at least one CMV antigen is administered about one, two, three, or four weeks after the administration of the CMV-HIV CAR T cells. In some embodiments, this can be in addition to administering the at least one CMV antigen or a nucleic acid molecule encoding at least one CMV antigen prior to the administration of the CMV-HIV CAR T cells, thus in some embodiments, the subject would be administered at least one CMV antigen or a nucleic acid molecule encoding at least one CMV antigen before the administration of the CMV-HIV CAR T cells and administered at least one CMV antigen or a nucleic acid molecule encoding at least one CMV antigen after the administration of the CMV-HIV CAR T cells.

A useful CMV vaccine comprises one or more CMV antigens or one or more nucleic acids encoding one or more CMV antigens. A CMV antigen can be any of a CMV protein, a fragment of a CMV protein, a modified CMV protein, a fragment of a modified CMV protein, a mutated CMV protein or a fragment thereof, or a fusion CMV protein or a fragment thereof. In some embodiments, a useful CVM vaccine comprises one or more nucleic acids encoding one or more CMV antigens. Examples of CMV antigens include pp65, IE1 exon 4 (IE1/e4), IE2 exon 5 (IE2/e5), fusions thereof, and antigenic fragments thereof, and variants thereof with 1, 2, 3, 4, or 5 amino acid modifications. In some embodiments, the 1, 2, 3, 4, or 5 amino acid modifications comprise 1-2 amino acid substitutions or 1-5 amino acid substitutions. In some embodiments, the amino acid substitutions are conservative. In some embodiments, a CMV antigen comprises a sequence selected from SEQ ID NOs: 57-64 and variants thereof having 1, 2, 3, 4, or 5 amino acid modifications. In some embodiments, the 1, 2, 3, 4, or 5 amino acid modifications comprise 1-2 amino acid substitutions or 1-5 amino acid substitutions. In some embodiments, the amino acid substitutions are conservative. In some embodiments, a CMV antigen can comprise a fragment of any of SEQ ID NOs: 57-64. The fragment can comprise of consist of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 contiguous amino acids of any of SEQ ID NOs: 57-64.

In various embodiments: a CMV antigen comprises a CMV protein or fragment thereof (e.g., CMVpp65 peptide); the nucleic acid molecule comprises a viral vector encoding (a) a CMV pp65 peptide or protein and (b) a fusion protein comprising exon 4 of CMV protein 1E1 (e4) and exon 5 of CMV protein 1E2 (e5). Non-limiting examples of nucleic acids encoding a CMV antigen include DNA, RNA, mRNA, vector, viral vector, lentiviral vector, MVA vector, bacterial artificial chromosome (BAC), vaccinia virus vector, adenovirus vector, adeno-associated virus vector, and others known in the art. Useful nucleic acids can encode one or more CMV antigens. In some embodiments, the nucleotide sequence for a CMV antigen is optimized.

In some embodiments, the at least one CMV antigen or a nucleic acid molecule encoding at least one CMV antigen is administered prior to or subsequent to administering the CMV-specific T cells in single or repeat dosing. In some embodiments, an effective amount of the at least one CMV antigen or a nucleic acid molecule encoding at least one CMV antigen is administered to a subject. In some embodiments, the at least one CMV antigen or a nucleic acid molecule encoding at least one CMV antigen is administered in an amount sufficient to illicit an immune response in a subject.

In some embodiments, the subject is also being treated with an anti-retroviral therapy (ART). In some embodiments, the ART regimen is reduced or stopped following administration of the CMV CAR T cells. In some embodiments, the ART regimen is temporarily stopped for 4 days prior to leukapheresis to collect peripheral blood mononuclear cells (PBMCs). In some embodiments, a subject resumes their ART regimen immediately after leukapheresis.

After leukapheresis, the apheresis product can be incubated overnight with one or more CMV antigens and/or CMV peptides. In some embodiments, the CMV-specific T cells are enriched based on interferon gamma (IFNγ) positivity. In some embodiments, the cells are then transduced with a self-inactivating lentiviral vector encoding a CAR described herein (e.g., vHIVR(N6)(EQ)BBζ-T2A-EGFRt_epHIV7; SEQ ID NO:44). In some embodiments, the population of CMV/HIV-CAR T cells is expanded in vitro in presence of IL-2, IL-15, and ART cocktail inhibitor for about 2 weeks. In some embodiments, the expanded population of CMV/HIV-CAR T cells is cryopreserved.

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In order that the invention described may be more fully understood, Examples are set forth. The materials and method used in the Examples below are detailed following the Examples. The Examples described in this application are offered to illustrate the methods and compositions provided herein and are not to be construed in any way as limiting their scope.

All publications, patent applications, patents, sequences, database entries, and other references mentioned herein are incorporated by reference in their entirety for any and all purposes.

Other features and advantages of the described compositions and methods will be apparent from the following detailed description and figures, and from the claims.

### BRIEF DESCRIPTION OF THE DRAWINGS

FIGS. 1A-D. Functional characterization of N6-CAR T cells. (A) Schematic diagram of the N6-CAR construct. The construct contains the GM-CSF receptor-α chain signal sequence (GMCSFRss) under the control of the EF1α promoter, the single-chain variable fragment (scFv) of the anti-gp120 bNAb N6 linked to the CD4 transmembrane (tm) and 4-1BB costimulatory domains through an IgG4 (EQ) spacer, followed by CD3ζ and a T2A linked truncated human EGFR (EGFRt). (B) Primary T cells were activated with CD3/CD28 microbeads and transduced with a lentiviral vector encoding N6-CAR. After ˜15-day in vitro expansion, CAR expression and T cell subsets were analyzed by flow cytometry using antibodies against EGFR, CD3, CD4 and CD8. Representative FACS plots of four HIV^{neg} donor-derived CAR T cell products are presented. (C) N6-CAR T cells or mock T cells derived from HIV^{neg} donors were cocultured with eGFP-expressing 8E5-gp120 cells at various total T cells:target (E:T) ratios for 96 hours followed by immunostaining for CD3 and eGFP. Cytotoxicity is calculated as follows: 100%−(% of remaining tumor cells in CAR T cell group/% of remaining tumor cells in mock T or negative target). n=3 donors. (D) N6-CAR T cell products were labeled with CellTrace™ Violet dye (CTV) and stimulated at an E:T ratio of 1:1 with 8,000 rads irradiated 8E5-gp120 for 8 days before CTV analysis (blue line). 8,000 rads irradiated lymphoblastoid cells that express the CD3 agonist OKT3 (LCL-OKT3) were used as positive control (red line), and media as negative control (black line). CTV dilutions from EGFR⁺ gated cells (lower panel) and EGFR⁻ gated cells (upper panel) are depicted. Representative data of five HIV^{neg} donor-derived CAR T cell products are presented.

FIGS. 2A-B: Development of 8E5-gp120 cell line. Parental 8E5 cells derive from HIV-infected lymphoblastic cells and carry a single, reverse transcriptase (RT)-defective copy of an integrated HIV genome. (A) Flow cytometry analysis using anti-gp120 monoclonal antibody staining showed ˜30% of the parental cells express gp120. (B) Cells were transduced with a lentiviral vector encoding eGFP and firefly luciferase (ffLuc) and then eGFP and gp120 double-positive cells were sorted and expanded for in vitro experiments.

FIG. 3: Specific cytotoxicity of N6-CAR T cells against gp120-positive cells. N6-CAR T cells derived from an HIVneg donor were co-cultured at various E:T ratios (1:1, 1:2 or 1:5) with eGPF+ 8E5 cells sorted for gp120 expression. Residual eGFP+tumor cells were measured by flow cytometry after 96 hours.

FIGS. 4A-B: Phenotypic characterization of N6-CAR T cells after stimulation with 8E5-gp120 cells. N6-CAR T cells derived from three HIVneg donors were cocultured at an E:T ratio of 1:1 with either 8E5-gp120 cells, LCL-OKT3 cells, or medium for 96 hours before flow cytometric analysis of the expression of (A) memory (CD62L, CD127 and CD27), or (B) exhaustion (LAG-3, PD-1 and Tim-3) markers.

FIG. 5: Specific binding of N6-scFv-Fc on gp120-expressing cells. 8E5-gp120 cells were stained with soluble N6 scFv-Fc at the indicated dilutions. Positive cells were quantified by flow cytometry. Staining with the anti-gp120 bNAb VRC01 was used as positive control, and gp120-negative KG-1a cells or staining with the secondary antibody alone were used as negative controls.

FIGS. 6A-F: Clinical scale manufacturing of CMV-HIV CAR T cells derived from HIV^{neg} and HIV^{pos} donors. (A) Manufacturing workflow to generate CMV-HIV CAR T cells as described in the Methods. (B) Representative FACS plots of CMV-specific T cells isolated from an HIV^{pos} donor before and after IFN-γ⁺ cell enrichment using the CliniMACS Prodigy® platform. (C) Flow cytometric analysis of the percentage of enriched IFN-γ+ CMV-specific T cells and their relative CD4 and CD8 expression. n=6-7 donors per group. (D) Growth curves of total cell number in final products derived from HIV^{neg} (n=6) and HIV^{pos} (n=7) donors over ˜15-day expansion. The expansion curves in presence of antiretroviral drugs (ARV, darunavir and enfuvirtide) are presented with red dotted lines. (E) Flow cytometric analysis of the percentage of CMV-HIV CAR T cell in the final cell products and their relative CD4 and CD8 expression. n=6-7 donors per group. (F) Total number of CMV-HIV CAR T cells in each final cell product.

FIG. 7: Memory cell subsets in CMV-specific T cells isolated from HIVneg and HIVpos donors. CMV-specific T cells (IFN-γ+CD3+) isolated from HIVneg and HIVpos donors were enriched using the CliniMACS Prodigy® system and immunostained with anti-CD27 and anti-CD45RA antibodies. Flow cytometric analysis show the percentage of CMV-specific T cells that are CD27+CD45RA+ stem cell memory T cells (Tscm), CD27+CD45RA− central memory (Tcm), CD27-CD45RA+ effector memory RA (TEMRA), and CD27-CD45RA-effector memory T cells (Tem). Lines indicate means±SD: n=5 donors per group.

FIGS. 8A-C: In vitro HIV replication, lentiviral transduction and cell expansion in presence of antiretroviral drugs (ARV). (A) The HIV protease inhibitor darunavir (D, EC50=4.3 nM) and the HIV fusion inhibitor enfurvitide (E, EC50=27.9 nM) prevent HIV replication when added in CD8+-depleted PBMCs infected with HIV-1 BaL (left panel) or HIV-1 NL4-3 (right panel) strains. (B) Darunavir (43 nM) and enfurvitide (279 nM) were added on the day of (Day 0) or 2 days after (Day 2) transduction of PBMCs with a lentiviral vector expressing eGFP. Flow cytometric analysis of eGFP expression 8 and 14 days after lentiviral transduction is shown. (C) Cell expansion of untransduced or transduced PBMCs in the presence or absence of antiretroviral drugs.

FIGS. 9A-D: Phenotypic characterization of CMV-HIV CAR T cell products derived from HIV^{neg} and HIV^{pos} donors. T cell memory markers: CD27⁺CD45RA⁺ stem cell memory T cells (Tscm), CD27⁺CD45RA⁻ central memory (Tcm), CD27⁻CD45RA⁺ effector memory RA (TEMRA), and CD27⁻CD45RA⁻ effector memory T cells (Tem) were analyzed by flow cytometry after INF-r enrichment (A) and in the final CAR T cell products (B). Exhaustion markers (LAG-3, PD-1 and Tim-3) were analyzed by flow cytometry in the final cell products (C) or within the EGFR+ CAR T cell fractions (D). Lines indicate means±SD; n=2-6 donors per group.

FIG. 10: Effector functions of CMV-HIV CAR T cells derived from HIV^{neg} donors. (A) Specific cytotoxicity against gp120-expressing target cells was determined by culturing CMV-HIV CAR T cell products with eGFP⁺ 8E5-gp120 or eGFP⁺ KG-1a cells at different E:T ratios 2:1, 1:1, 1:2 or 1:5) for 96 hours followed by immunostaining for CD3 and eGFP. Percentages of remaining eGFP⁺ tumor cells were measured by flow cytometry and cytotoxicity was calculated. (B) CMV-HIV CAR T cell products were labeled with CTV and cultured for 8 days with CMVpp65 peptide-pulsed and 3,500 rads irradiated PBMCs (CMVpp65-PBMC), 8,000 rads irradiated LCL-OKT3 or KG-1a cells or media. CMV-HIV CAR T cell proliferation was determined by CTV dilution. Representative data of four donors are shown. (C) CMV-HIV CAR T cell or CMV-specific T-cell products derived from the same donor were stimulated overnight with CMVpp65 peptide-pulsed autologous PBMC (CMVpp65-PBMC), LCL-OKT3, 8E5-gp120, KG-1a cells or media. Cocultures were stained for surface CD8 followed by intracellular IFN-γ expression. Representative data of three different donors are shown.

FIG. 11: Effector functions of CMV-HIV CAR T cells derived from HIV^{pos} donors. (A) Representative FACS plots of CMV-specific T cells enriched from an HIV^{pos} donor and transduced with a lentiviral vector expressing N6-CAR (n=7). The transduction efficiency was assessed on Day 7 by measuring EGFR expression in T cells. The CMV-HIV CAR T cell products were then stimulated overnight with CMVpp65 peptide-pulsed autologous PBMC and analyzed by flow cytometry for the expression of IFN-γ, CD3, CD4 and CD8. Representative data of three different donors (n=3) are shown. (B-C) Specific cytotoxicity was determined by co-culturing CMV-HIV CAR T cells (n=3) with eGFP+ 8E5-gp120 or eGFP⁺ KG-1a cells at different E:T ratios (2:1, 1:1, or 1:5) for 24 hours (n=2) and 96 hours (n=3) followed by immunostaining for CD3, EGFR as well as LAG-3, PD-1 and Tim-3 exhaustion markers. Percentages of remaining eGFP+ tumor cells were measured by flow cytometry and cytotoxicity was calculated as described in Methods. The graph presents the cytotoxicity of CAR T cells from two or three donors against 8E5-gp120 target cells at different E:T ratios. (D) CMV-HIV CAR T cells or CMV-CD19 CAR T cells were manufactured from the same HIV^{pos} donor and cultured with HIV_{NL4-3}-infected eGFP⁺ Jurkat cells at different E:T ratios (1:1, 1:2 and 1:4) for 7 days. The cytotoxicity of the CAR T cell products against HIV_{NL4-3}-infected eGFP⁺ Jurkat cells was calculated and normalized to an untreated control well. The levels of HIV p24 in the cell supernatants on Day 7 were measured by ELISA and normalized to the p24 levels in the control condition at an E:T ratio of 1:1 (E). (F) CMV-HIV CAR T cells and CMV-CD19 CAR T cells were manufactured from an HIV^{pos} donor on ART. Levels of p24 were measured in the culture supernatant by ELISA after 20 day-expansion and normalized to the p24 level in supernatant of CMV-CD19 CAR T cells. Data from one HIV^{pos} donor are shown in (D), (E), and (F).

FIG. 12: CMVpp65-driven expansion of CMV-HIV CAR T cells and dose-dependent control of HIV viremia in hu-PBMC-NSG mice on ART. (A) NSG humanized peripheral blood mononuclear cells (hu-PBMC) mouse model of HIV on ART and experimental design. HIV-infected mice on oral ART were treated with a low dose CMV-HIV CAR T cells (0.1×10⁶ EGFR+ T cells), with or without CMVpp65 vaccine, or high dose of CMV-HIV CAR T cells (1×10⁶ EGFR+ T cells) with CMVpp65 vaccine on Day 28. Mice treated with CMV-negative T cells (1×10⁶ cells) from the same HIV^{neg} donor, with or without CMVpp65 vaccine, were used as controls. (B) HIV viral load in the peripheral blood on Day 28. Additive models with the baseline HIV viral load (Day 21), log₁₀ CD3, log₁₀ CD4 and treatment group were considered, and the best model was chosen based on Akaike information criteria. This model included treatment groups only so analysis of variance followed by the Tukey method for all possible one-sided comparisons (Family wise error rate (FWER)=0.05) was used to assess if there were treatment differences among the control (T-cell treated mice), low dose and high dose CMV-HIV CAR T cell-treated cohorts; ***P-value<0.001; **P-value=0.002; n=8-17/group. (C) Flow cytometric analysis of EGFR+ CAR T cell expansion in the peripheral blood between Day 33 and Day 42. n=8/group. Note that one female mouse in the “Low dose CAR T+vaccine group” did not have a Day 42 measure and was not included in this analysis. (D) HIV viral load in the peripheral blood on Day 42, after vaccine stimulation and ART interruption. The best model was an analysis of covariance including log₁₀ CD3, log₁₀ CD4 and treatment group followed by the Tukey method for all possible one-sided comparisons. *P-value<0.01, *P-value=0.03; n=6-8/group. (E) Flow cytometric analysis of the frequency of EGFR⁺ CAR T cells in the bone marrow at the time of sacrifice. The data was transformed using a logit transformation. ANOVA followed by the Tukey method for all possible one-sided comparisons (Family wise error rate (FWER)=0.05) was used to assess if there were treatment differences among the CAR T cell-treated cohorts; *P-value<0.01; *P-value=0.02; n=7-8/group. (F) Percentage of EGFR⁺ CAR T cells in the bone marrow plotted against the percentage of p24⁺ T cells in the bone marrow. Staining for surface antibodies (CD45, CD3, and EGFR) were performed as in panel (C), while intracellular p24 HIV-1 antigen was stained with KC57-FITC antibody after fixation and permeabilization. The simple least squares model with only % of EGFR⁺ CAR T cells was best, both % of EGFR⁺ CAR T cells and % of p24⁺ T cells were transformed using a logit. Box and whisker plots were used to present the data in (B), (D) and (E). The black box represents the quartiles and black line represent the quartiles and median and the plus sign represents the mean, and values outside the whiskers are considered outliers.

FIG. 13: Body weight and temperature in HIV-infected hu-PBMC-NSG mice treated with ART, CMV-HIV CAR T cells, with or without CMVpp65 vaccine. Body weight (A) and temperature (B) were monitored weekly in the hu-PBMC mouse model upon transplant with HIVneg donor-derived PBMCs (Day 0). Mice started oral ART regimen on Day 12, received a single IV dose of CMV-HIV CAR T cells (low [0.1×106] or high [1×10⁶] dose) on Day 21 and CMVpp65 vaccine on Day 28. No statistical significance between the groups was observed using ANOVA mixed-effects analysis. Group sizes are as follows: “T cells” n=8 (4F4M), “T cells+vaccine” n=8 (4F4M), “Low dose CAR T cells” n=8 (4F4M), “Low dose CAR T cells+vaccine” n=8 (4F4M), “High dose CAR T cells+vaccine” n=9 (4F5M).

FIG. 14: EGFR+ CAR T cell expansion in the peripheral blood between Day 33 and Day 42 in HIV-infected hu-PBMC-NSG mice treated with ART, CMV-HIV CAR T cells, with or without CMVpp65 vaccine. EGFR+ CAR T cell expansion in the peripheral blood was assessed based on the mean slopes of the linear regression lines for EGFR+ CAR T cell number/μL using a log 10 transformation from Day 33 and Day 42. Statistical significance was determined using one-sided Tukey contrasts *P-value=0.03; *P-value=0.02. n=8/group, same groups as in FIG. 6C.

FIG. 15: Distribution and phenotype of HIV^{pos} donor-derived CMV-HIV CAR T cells in humanized PBMC-NSG mouse model. Flow cytometric analyses of EGFR+ CAR T cells 6 weeks post-CAR T cell infusion. (A) Frequency of CD4⁺ and CD8⁺ T cells, (B) CD62L⁺ and (C) CD27⁺ within the EGFR⁺ CAR T cell fraction. Lines represent means±SD; n=5.

FIG. 16: Schema of the clinical trial timeline. After screening and signing patients up for the trial, participants will interrupt their ART regimen for 4 days prior to leukapheresis to prevent inhibition of lentiviral transduction of the T cells during CAR T cell manufacturing. Participants will resume their ART regimen immediately after leukapheresis. Once the CMV-HIV T cell population is prepared, participants will receive 5×10⁶ cells, 25×10⁸ cells, or 50×10⁶ cells (day 0). Dose limiting toxicity (DLT) will be evaluated beginning from the day prior to the T cell infusion (day −1) for 60 days following the infusion. Blood will be drawn and evaluated on the days indicated on the timeline followed by long term follow up (LTFU).

FIGS. 17A-E. Schema of the HIVR(N6)(EQ)BBZ-T2A-EGFRt_epHIV7 plasmid. Map (A) and Sequence (D-E) of HIVscFv(N6)-IgG4(L235E,N297Q)-41BB-Zeta(CO)-T2A-EGFRt_epHIV7 (10008 bp; SEQ ID NO: 44) lentiviral vector.

### DETAILED DESCRIPTION

The studies described herein show that CMV-specific T cells expressing a CAR targeted to HIV (CMV-CAR T cells) exhibit dual effector functions upon in vitro stimulation through their endogenous CMV-specific T cell receptors or the introduced CAR. The studies described herein using a humanized HIV mouse model show that CMV vaccination during ART accelerates CMV-HIV CAR T cell expansion in the peripheral blood and that higher numbers of CMV-HIV CAR T cells are associated with a better control of HIV viral load and fewer HIV antigen p24+ cells in the bone marrow upon ART interruption. The CMV-CAR T cells and CMV antigens can be used to treat subjects infected with HIV.

### I. Chimeric Antigen Receptors

A chimeric antigen receptor (CAR) refers to an artificial immune cell receptor that is engineered to recognize and bind to a surface antigen. A T cell that expresses a CAR polypeptide is referred to as a CAR T cell. CAR have the ability to redirect T-cell specificity and reactivity toward a selected target in a non-MHC-restricted manner. The non-MHC-restricted antigen recognition gives CAR T cells the ability to recognize an antigen independent of antigen processing, thereby bypassing a major mechanism of tumor escape. A CAR can also be expressed by other immune effector cells, including but not limited to natural killer CAR (“NK CAR”) and directed NK cell killing to cells expressing the target of the CAR.

There are various generations of CARs, each of which contains different components. First generation CARs join an antibody-derived scFv to the CD3ζ intracellular signaling domain of the T cell receptor through a spacer region (also called a hinge domain) and a transmembrane domain. Second generation CARs incorporate an additional co-stimulatory domain (e.g., CD28, 4-BB, or ICOS) to supply a co-stimulatory signal. Third generation CARs contain two co-stimulatory domains (e.g., a combination of CD27, CD28, 4-1BB, ICOS, or OX40) fused with the TCR CD3ζ chain.

There can be a spacer between the co-stimulatory domain and the CD3ζ domain, but this is optional. A CAR is often fused to a signal peptide at the N-terminus for surface expression. In some cases the CAR can be co-expressed with a polypeptide that can serve as marker, for example a truncated EGFR receptor lacking signaling function or a truncated CD19 receptor lacking signaling function.

The Examples described herein relate to an HIV CAR having the sequence:

(SEQ ID NO: 10)
RAHLVQSGTAMKKPGASVRVSCQTSGYTFTAHILFWFRQAPGRGLEWVG
WIKPQYGAVNFGGGFRDRVTLTRDVYREIAYMDIRGLKPDDTAVYYCAR
DRSYGDSSWALDAWGQGTTVVVSAGGGSGGGSGGGSGGGSYIHVTQSPS
SLSVSIGDRVTINCQTSQGVGSDLHWYQHKPGRAPKLLIHHTSSVEDGV
PSRFSGSGFHTSFNLTISDLQADDIATYYCQVLQFFGRGSRLHIKESKY
GPPCPPCPAPEFEGGPSVFLFPPKPKDTLMISRTPEVTCVVVDVSQEDP
EVQFNWYVDGVEVHNAKTKPREEQFQSTYRVVSVLTVLHQDWLNGKEYK
CKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQ
EGNVFSCSVMHEALHNHYTQKSLSLSLGKMALIVLGGVAGLLLFIGLGI
FFKRGRKKLLYIFKQPFMRPVQTTQEEDGCSCRFPEEEEGGCELGGGRV
KFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDT
YDALHMQALPPR

From amino terminus to carboxy terminus the CAR includes a VH domain having the sequence:

(SEQ ID NO: 8)
RAHLVQSGTAMKKPGASVRVSCQTSGYTFTAHILFWFRQAPGRGLEWVG
WIKPQYGAVNFGGGFRDRVTLTRDVYREIAYMDIRGLKPDDTAVYYCAR
DRSYGDSSWALDAWGQGTTVVVSA;

a linker having the sequence:

(SEQ ID NO: 11)
GGGSGGGSGGGSGGGS;

a VL domain having the sequence:

(SEQ ID NO: 7)
YIHVTQSPSSLSVSIGDRVTINCQTSQGVGSDLHWYQHKPGRAPKLLIH
HTSSVEDGVPSRFSGSGFHTSFNLTISDLQADDIATYYCQVLQFFGRGS
RLHIK;

a spacer domain having the sequence:

(SEQ ID NO: 33)
ESKYGPPCPPCPAPEFEGGPSVFLFPPKPKDTLMISRTPEVTCVVVDVS
QEDPEVQFNWYVDGVEVHNAKTKPREEQFQSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK;

a CD4 transmembrane domain having the sequence:

(SEQ ID NO: 18)
MALIVLGGVAGLLLFIGLGIFF;

a 41-BB co-stimulatory domain having the sequence:

(SEQ ID NO: 38)
KRGRKKLLYIFKQPFMRPVQTTQEEDGCSCRFPEEEEGGCEL;

a linker having the sequence:
GGG; and
a CD3ζ domain having the sequence:

(SEQ ID NO: 35)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In some embodiments, the CAR sequence can be preceded by a GMCSFRa signal peptide having the sequence:

MLLLVTSLLLCELPHPAFLLIP.

In some embodiments, the CAR sequence can be followed by a T2A skip sequence having the sequence:

(SEQ ID NO: 45)
LEGGGEGRGSLLTCGDVEENPGPR;

a GMCSFRa signal peptide having the sequence:

MLLLVTSLLLCELPHPAFLLIP;

a truncated EGFR receptor lacking signaling activity and having the sequence:

(SEQ ID NO: 46)
RKVCNGIGIGEFKDSLSINATNIKHFKNCTSISGDLHILPVAFRGDSFT
HTPPLDPQELDILKTVKEITGFLLIQAWPENRTDLHAFENLEIIRGRTK
QHGQFSLAVVSLNITSLGLRSLKEISDGDVIISGNKNLCYANTINWKKL
FGTSGQKTKIISNRGENSCKATGQVCHALCSPEGCWGPEPRDCVSCRNV
SRGRECVDKCNLLEGEPREFVENSECIQCHPECLPQAMNITCTGRGPDN
CIQCAHYIDGPHCVKTCPAGVMGENNTLVWKYADAGHVCHLCHPNCTYG
CTGPGLEGCPTNGPKIPSIATGMVGALLLLLVVALGIGLFM.

### (a) Extracellular Binding Domain

Useful HIV CAR described herein are fusion proteins comprising an extracellular binding domain that recognizes HIV. This extracellular domain is often a single chain fragment (scFv) of an antibody or other antibody fragment, but it can also be a ligand that binds to an HIV protein.

Where the binding domain is an scFv, there is a heavy chain variable region and a light chain variable region, which can be in an order and are joined together via a flexible linker of, e.g., 5-25 amino acids. In some embodiments, a useful flexible linker is 1, 2, 3, 4, 5, 6, 7, 8, 9, or 10 repeats of the sequence GGGS (SEQ ID NO: 13). In some embodiments, a useful flexible linker is 1, 2, 3, 4, 5, 6, 7, 8, 9, or 10 repeats of the sequence GGGGS (SEQ ID NO: 14). In some embodiments, the light chain variable domain is amino terminal to the heavy chain variable domain in other cases it is carboxy terminal to the heavy chain variable domain. In some cases the linker comprises the sequence SSGGGGSGGGGSGGGGS (SEQ ID NO:12).

Described herein a studies using a HIV CAR that includes the VH and VL domains of N6, a broadly neutralizing antibody that binds the CD4-binding site of HIV Env that potently neutralizes 98% of HIV-1 isolates including 16 of 20 that evolved to circumvent common mechanisms of resistance¹⁸. The CDRs in the VL and VH domains are underlined in the sequences below.

| | | |
|---|---|---|
| N6 | VL | YIHVTQSPSSLSVSIGDRVTINCQTSQGVGSDLHWYQH |
| | | KPGRAPKLLIHHTSSVEDGVPSRFSGSGFHTSFNLTIS |
| | | DLQADDIATYYCQVLQFFGRGSRLHIK |
| | | (SEQ ID NO: 7) |
| | VH | RAHLVQSGTAMKKPGASVRVSCQTSGYTFTAHILFWFR |
| | | QAPGRGLEWVGWIKPQYGAVNFGGGFRDRVTLTRDVYR |
| | | EIAYMDIRGLKPDDTAVYYCARDRSYGDSSWALDAWGQ |
| | | GTTVVVSA (SEQ ID NO: 8) |

Thus, the svFv in an HIV CAR can include a VL domain that is 95% identical to YIHVTQSPSSLSVSIGDRVTINCQTSQGVGSDLHWYQHKPGRAPKLLIHHTSSVEDGVPSR FSGSGFHTSFNLTISDLQADDIATYYCQVLQFFGRGSRLHIK (SEQ ID NO:7) and includes the following CDR sequences: QTSQGVGSDLH (VL-CDR1; SEQ ID NO:1), HTSSVED (VL-CDR2; SEQ ID NO:2), and QVLQF (VL-CDR3; SEQ ID NO:3) and a VH domain that is 95% identical to RAHLVQSGTAMKKPGASVRVSCQTSGYTFTAHILFWFRQAPGRGLEWVGWIKPQYGAVN FGGGFRDRVTLTRDVYREIAYMDIRGLKPDDTAVYYCARDRSYGDSSWALDAWGQGTTVV VSA (SEQ ID NO:8) and includes the following CDR sequences AHILF (VH-CDR1; SEQ ID NO:4) WIKPQYGAVNFGGGFRD (VH-CDR2; SEQ ID NO:5), and DRSYGDSSWALDA (VH-CDR3; SEQ ID NO:6).

The N6 scFv used in the HIV CAR described herein has the sequence:

(SEQ ID NO: 9)
RAHLVQSGTAMKKPGASVRVSCQTSGYTFTAHILFWFRQAPGRGLEWVG
WIKPQYGAVNFGGGFRDRVTLTRDVYREIAYMDIRGLKPDDTAVYYCAR
DRSYGDSSWALDAWGQGTTVVVSAGGGSGGGSGGGSGGGSYIHVTQSPS
SLSVSIGDRVTINCQTSQGVGSDLHWYQHKPGRAPKLLIHHTSSVEDGV
PSRFSGSGFHTSFNLTISDLQADDIATYYCQVLQFFGRGSRLHIK

### (b) Transmembrane Domain

The CAR polypeptides disclosed herein can contain a transmembrane domain, which can be a hydrophobic alpha helix that spans the membrane. As used herein, a transmembrane domain refers to any protein structure that is thermodynamically stable in a cell membrane, preferably a eukaryotic cell membrane.

The transmembrane domain of the HIV CAR used in the Examples has CD4 transmembrane domain having the sequence: MALIVLGGVAGLLLFIGLGIFF (SEQ ID NO: 18). Other transmembrane domains can be used including those shown below.

TABLE 1
Examples of Transmembrane Domains

| Name | Accession | Length | Sequence |
|---|---|---|---|
| CD3z | J04132.1 | 21 aa | LCYLLDGILFIYGVILTALFL (SEQ ID NO: 15) |
| CD28 | NM_006139 | 27 aa | FWVLVVVGGVLACYSLLVTVAFIIFWV (SEQ ID NO: 16) |
| CD28(M) | NM_006139 | 28 aa | MFWVVLVVVGGVLACYSLLVTVAFIIFWWV (SEQ ID NO: |
| | | | 17) |
| CD4 | M35160 | 22 aa | MALIVLGGVAGLLLFIGLGIFF (SEQ ID NO: 18) |
| CD8tm | NM_001768 | 21 aa | IYIWAPLAGTCGVLLLSLVIT (SEQ ID NO: 19) |
| CD8tm2 | NM_001768 | 23 aa | IYIWAPLAGTCGVLLLSLVITLY (SEQ ID NO: 20) |
| CD8tm3 | NM_001768 | 24 aa | IYIWAPLAGTCGVLLLSLVITLYC (SEQ ID NO: 21) |
| 4-1BB | NM_001561 | 27 aa | ISFFLALTSTALLFLLFFLTLRFSVV (SEQ ID NO: 22) |
| NKG2D | NM_007360 | 21 aa | PFFFCCFIAVAMGIRFIIMVA (SEQ ID NO: 23) |

### (c) Spacer Domain

The CAR or polypeptide described herein can include a spacer domain located between the HIV targeting domain (i.e., an HIV targeted scFv or variant thereof) and the transmembrane domain. The spacer region can function to provide flexibility to the CAR, or domains thereof, or to prevent steric hindrance of the CAR, or domains thereof. A variety of different spacers can be used. Some of them include at least portion of a human Fc region, for example a hinge portion of a human Fc region or a CH3 domain or variants thereof. Table 2 below provides various spacer domains that can be used in the CARs described herein.

TABLE 2
Examples of Spacer Domains

| Name | Length | Sequence |
|---|---|---|
| a3 | 3 aa | AAA |
| linker | 10 aa | GGGSSGGGSG (SEQ ID NO: 24) |
| lgG4 hinge (SP) | 12 aa | ESKYGPPCPPCP (SEQ ID NO: 25) |
| (S228P) | | |
| IgG4 hinge | 12 aa | ESKYGPPCPSCP (SEQ ID NO: 26) |
| IgG4 hinge (S228P) + | 22 aa | ESKYGPPCPPCPGGGSSGGGSG (SEQ ID NO: 27) |
| linker | | |
| CD28 hinge | 39 aa | IEVMYPPPYLDNEKSNGTIIHVKGKHLCPSPLFPGPSKP |
| | | (SEQ ID NO: 28) |
| CD8 hinge-48 aa | 48 aa | AKPTTTPAPRPPTPAPTIASQPLSLRPEACRPAAGGAV |
| | | HTRGLDFACD (SEQ ID NO: 29) |
| CD8 hinge-45 aa | 45 aa | TTTPAPRPPTPAPTIASQPLSLRPEACRPAAGGAVHTR |
| | | GLDFACD (SEQ ID NO: 30) |
| IgG4(HL-CH3) | 129 aa | ESKYGPPCPPCPGGGSSGGGSGGQPREPQVYTLPPS |
| Also called IgG4 | | QEEMTKNQVSLTCLVKGFYPSDIAVEWESNGQPENNY |
| (HL-ΔCH2) | | KTTPPVLDSDGSFFLYSRLTVDKSRWQEGNVFSCSVM |
| (includes S228P in hinge) | | HEALHNHYTQKSLSLSLGK (SEQ ID NO: 31) |
| IgG4 | 229 aa | ESKYGPPCPSCPAPEFEGGPSVFLFPPKPKDTLMISRT |
| (L235E, N297Q) | | PEVTCVVVDVSQEDPEVQFNWYVDGVEVHNAKTKPRE |
| | | EQFQSTYRVVSVLTVLHQDWLNGKEYKCKVSNKGLPS |
| | | SIEKTISKAKGQPREPQVYTLPPSQEEMTKNQVSLTCLV |
| | | KGFYPSDIAVEWESNGQPENNYKTTPPVLDSDGSFFLY |
| | | SRLTVDKSRWQEGNVFSCSVMHEALHNHYTQKSLSLS |
| | | LGK (SEQ ID NO: 32) |
| IgG4 | 229 aa | ESKYGPPCPPCPAPEFEGGPSVFLFPPKPKDTLMISRT |
| (S228P, L235E, N297Q) | | PEVTCVVVDVSQEDPEVQFNWYVDGVEVHNAKTKPRE |
| | | EQFQSTYRVVSVLTVLHQDWLNGKEYKCKVSNKGLPS |
| | | SIEKTISKAKGQPREPQVYTLPPSQEEMTKNQVSLTOLV |
| | | KGFYPSDIAVEWESNGQPENNYKTTPPVLDSDGSFFLY |
| | | SRLTVDKSRWQEGNVFSCSVMHEALHNHYTQKSLSLS |
| | | LGK (SEQ ID NO: 33) |
| IgG4(CH3) | 107 aa | GQPREPQVYTLPPSQEEMTKNQVSLTCLVKGFYPSDIA |
| Also called IgG4(ACH2) | | VEWESNGQPENNYKTTPPVLDSDGSFFLYSRLTVDKS |
| | | RWQEGNVFSCSVMHEALHNHYTQKSLSLSLGK (SEQ |
| | | ID NO: 34) |

Some spacer domains include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer domains include an immunoglobulin CH3 domain (called CH3 or ACH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The spacer domain can also comprise an IgG4 hinge region having the sequence ESKYGPPCPSCP (SEQ ID NO: 26) or ESKYGPPCPPCP (SEQ ID NO: 25). The hinge/linger region can also comprise the sequence ESKYGPPCPPCP (SEQ ID NO: 3) followed by the linker sequence GGGSSGGGSG (SEQ ID NO: 24) followed by IgG4 CH3 sequence:

(SEQ ID NO: 34)
GQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQ
KSLSLSLGK.

Thus, the spacer domain can comprise the sequence: ESKYGPPCPPCPGGGSSGGGSGGQPREPQVYTLPPSQEEMTKNQVSLTCLVKGFYPSDI AVEWESNGQPENNYKTTPPVLDSDGSFFLYSRLTVDKSRWQEGNVFSCSVMHEALHNHY TQKSLSLSLGK (SEQ ID NO: 31). In some cases, the spacer has 1, 2, 3, 4, or 5 single amino acid changes (e.g., conservative changes) compared to SEQ ID NO: 31. In some cases, the IgG4 Fc hinge/linker region that is mutated at two positions (L235E; N297Q) in a manner that reduces binding by Fc receptors (FcRs).

### (d) Intracellular Signaling Domains

Any of the CAR constructs described herein contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three immunoreceptor tyrosine-based activation motifs (ITAMs), which transmit an activation signal to the T cell after the T cell is engaged with a cognate antigen. In some cases, CD3ζ provides a primary T cell activation signal but not a fully competent activation signal, which requires a co-stimulatory signal.

Accordingly, in some examples, the CAR polypeptides disclosed herein may further comprise one or more co-stimulatory signaling domains in addition to CD3ζ. For example, the co-stimulatory domain CD28 and/or 4-1BB can be used to transmit a proliferative/survival signal together with the primary signaling mediated by CD3ζ.

The co-stimulatory domain(s) are located between the transmembrane domain and the CD3ζ signaling domain. Table 3 includes examples of suitable co-stimulatory domains together with the sequence of the CD3ζ signaling domain.

TABLE 3
CD3Z Domain and Examples of Co-stimulatory Domains

| Name | Accession | Length | Sequence |
|---|---|---|---|
| CD3ζ | J04132.1 | 113 aa | RVKFSRSADAPAYQQGQNQLYNELNLGRREEYDVLDKRR |
| | | | GRDPEMGGKPRRKNPQEGLYNELQKDKMAEAYSEIGMKG |
| | | | ERRRGKGHDGLYQGLSTATKDTYDALHMQALPPR (SEQ |
| | | | ID NO: 35) |
| | | | ITAMS 1-3 underlined |
| CD3ζ | | 113 aa | RVKFSRSADAPAYQQGQNQLYNELNLGRREEYDVLDKRR |
| variant | | | GRDPEMGGKPRRKNPQEGLFNELQKDKMAEAFSEIGMKG |
| | | | ERRRGKGHDGLFQGLSTATKDTFDALHMQALPPR (SEQ ID |
| | | | NO: 50) |
| CD3ζ | | 113 aa | RVKFSRSADAPAYQQGQNQLYNELNLGRREEYDVLDKRR |
| variant | | | GRDPEMGGKPRRKNPQEGLFNELQKDKMAEAFSEIGMKG |
| | | | ERRRGKGHDGLYQGLSTATKDTYDALHMQALPPR (SEQ |
| | | | ID NO: 51) |
| CD3ζ | | 113 aa | RVKFSRSADAPAYQQGQNQLYNELNLGRREEYDVLDKRR |
| variant | | | GRDPEMGGKPRRKNPQEGLYNELQKDKMAEAYSEIGMKG |
| | | | ERRRGKGHDGLFQGLSTATKDTFDALHMQALPPR (SEQ ID |
| | | | NO: 52) |
| CD3ζ | | 113 aa | RVKFSRSADAPAYQQGQNQLYNELNLGRREEYDVLDKRR |
| variant | | | GRDPEMGGKPRRKNPQEGLYNELQKDKMAEAFSEIGMKG |
| | | | ERRRGKGHDGLFQGLSTATKDTFDALHMQALPPR (SEQ ID |
| | | | NO: 53) |
| CD3ζ | | 113 aa | RVKFSRSADAPAYQQGQNQLYNELNLGRREEYDVLDKRR |
| variant | | | GRDPEMGGKPRRKNPQEGLFNELQKDKMAEAYSEIGMKG |
| | | | ERRRGKGHDGLFQGLSTATKDTFDALHMQALPPR (SEQ ID |
| | | | NO: 54) |
| CD3ζ | | 113 aa | RVKFSRSADAPAYQQGQNQLYNELNLGRREEYDVLDKRR |
| variant | | | GRDPEMGGKPRRKNPQEGLFNELQKDKMAEAFSEIGMKG |
| | | | ERRRGKGHDGLYQGLSTATKDTFDALHMQALPPR (SEQ ID |
| | | | NO: 55) |
| CD3ζ | | 113 aa | RVKFSRSADAPAYQQGQNQLYNELNLGRREEYDVLDKRR |
| variant | | | GRDPEMGGKPRRKNPQEGLFNELQKDKMAEAFSEIGMKG |
| | | | ERRRGKGHDGLFQGLSTATKDTYDALHMQALPPR (SEQ ID |
| | | | NO: 56) |
| CD28 | NM_006139 | 42 aa | RSKRSRLLHSDYMNMTPRRPGPTRKHYQPYAPPRDFAAY |
| | | | RS (SEQ ID NO: 36) |
| CD28gg* | NM_006139 | 42 aa | RSKRSRGGHSDYMNMTPRRPGPTRKHYQPYAPPRDFAAY |
| | | | RS (SEQ ID NO: 37) |
| 41BB | NM_001561 | 42 aa | KRGRKKLLYIFKQPFMRPVQTTQEEDGCSCRFPEEEEGGC |
| | | | EL (SEQ ID NO: 38) |
| OX40 | NM_003327 | 42 aa | ALYLLRRDQRLPPDAHKPPGGGSFRTPIQEEQADAHSTLA |
| | | | KI (SEQ ID NO: 39) |
| 2B4 | NM_016382 | 120 aa | WRRKRKEKQSETSPKEFLTIYEDVKDLKTRRNHEQEQTFP |
| | | | GGGSTIYSMIQSQSSAPTSQEPAYTLYSLIQPSRKSGSRKR |
| | | | NHSPSFNSTIYEVIGKSQPKAQNPARLSRKELENFDVYS |
| | | | (SEQ ID NO: 40) |

In some examples, the CD3ζ signaling domain comprises an amino acid sequence that is at least 90%, at least 95%, at least 98% identical to SEQ ID NO: 35. In such instances, the CD3ζ signaling domain has 1, 2, 3, 4, or 5 amino acid changes (preferably conservative substitutions) compared to SEQ ID NO: 35. In other examples, the CD3ζ signaling domain is SEQ ID NO: 35.

In various embodiments: the co-stimulatory domain is selected from the group consisting of: a co-stimulatory domain depicted in Table 3 or a variant thereof having 1-5 (e.g., 1 or 2) amino acid modifications, a CD28 co-stimulatory domain or a variant thereof having 1-5 (e.g., 1 or 2) amino acid modifications, a 4-1BB co-stimulatory domain or a variant thereof having 1-5 (e.g., 1 or 2) amino acid modifications and an OX40 co-stimulatory domain or a variant thereof having 1-5 (e.g., 1 or 2) amino acid modifications. In certain embodiments, a 4-1BB co-stimulatory domain or a variant thereof having 1-5 (e.g., 1 or 2) amino acid modifications is present in the CAR polypeptides described herein.

In some embodiments, there are two co-stimulatory domains, for example, a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. In various embodiments, the co-stimulatory domain is amino terminal to the CD3ζ signaling domain and a short linker consisting of 2-10, e.g., 3 amino acids (e.g., GGG) is can be positioned between the co-stimulatory domain and the CD3ζ signaling domain.

In some cases, the CAR can be produced using a vector in which the CAR open reading frame is followed by a T2A ribosome skip sequence and a truncated EGFR (EGFRt), which lacks the cytoplasmic signaling tail, or a truncated CD19R (also called CD19t). In this arrangement, co-expression of EGFRt or CD19t provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt or the CD19t incorporated in the CAR lentiviral vector can act as suicide gene to ablate the CAR+ T cells in cases of treatment-related toxicity.

The CD3ζ signaling domain can be followed by a ribosomal skip sequence (e.g., LEGGGEGRGSLLTCGDVEENPGPR; SEQ ID NO: 45) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPVAFRGD SFTHTPPLDPQELDILKTVKEITGFLLIQAWPENRTDLHAFENLEIIRGRTKQHGQFSLAWSL NITSLGLRSLKEISDGDVIISGNKNLCYANTINWKKLFGTSGQKTKIISNRGENSCKATGQVC HALCSPEGCWGPEPRDCVSCRNVSRGRECVDKCNLLEGEPREFVENSECIQCHPECLPQ AMNITCTGRGPDNCIQCAHYIDGPHCVKTCPAGVMGENNTLVWKYADAGHVCHLCHPNCT YGCTGPGLEGCPTNGPKIPSIATGMVGALLLLLWALGIGLFM (SEQ ID NO: 46). In some cases, the truncated EGFR has 1, 2, 3, 4 of 5 amino acid changes (preferably conservative) compared to SEQ ID NO: 46.

Alternatively the CD3ζ signaling domain can be followed by a ribosomal skip sequence (e.g., LEGGGEGRGSLLTCGDVEENPGPR; SEQ ID NO: 45) and a truncated CD19R (also called CD19t) having a sequence that is at least 90%, at least 95%, at least 98% identical to or identical to:

(SEQ ID NO: 47)
MPPPRLLFFLLFLTPMEVRPEEPLVVKVEEGDNAVLQCLKGTSDGPTQQ
LTWSRESPLKPFLKLSLGLPGLGIHMRPLAIWLFIFNVSQQMGGFYLCQ
FRWNVSDLGGLGCGLKNRSSEGPSSPSGKLMSPKLYVWAKDRPEIWEGE
PPGPPSEKAWQPGWTVNVEGSGELPCVPPRDSLNQSLSQDLTMAPGSTL
WLSCGVPPDSVSRGPLSWTHVHPKGPKSLLSLELKDDRPARDMWVMETG
LLLPRATAQDAGKYYCHRGNLTMSFHLEITARPVLWHWLLRTGGWKVSA
VTLAYLIFCLCSLVGILHLQRALVLRRKR.

The CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte, and most preferably an autologous T lymphocyte.

Various T cell subsets isolated from the patient can be transduced with a vector for CAR or polypeptide expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of the CAR or as well as a non-immunogenic surface marker for in vivo detection, ablation, and potential ex vivo selection. The activated/genetically modified central memory T cells can be expanded in vitro with IL-2/IL-15 and then cryopreserved. Additional methods of preparing CAR T cells can be found in PCT/US2016/043392.

Methods for preparing useful T cell populations are described in, for example, WO 2017/015490 and WO 2018/102761. In some cases, it may be useful to use natural killer (NK) cells, e.g., allogenic NK cells derived from peripheral blood or cord blood. In other cases, NK cells can be derived from human embryonic stem cells (hESCs) or induced pluripotent stem cells (iPSCs).

In some embodiments, described herein is a composition comprising the iPSC-derived CAR T cells or CAR NK cells. In some embodiments, a composition comprising iPSC-derived CAR T cells or CAR NK cells has enhanced therapeutic properties. In some embodiments, the iPSC-derived CAR T cells or CAR NK cells demonstrate enhanced functional activity including potent cytokine production, cytotoxicity and cytostatic inhibition of tumor growth, e.g., as activity that reduces the amount of tumor load.

The CAR can be transiently expressed in a T cell population by an mRNA encoding the CAR. The mRNA can be introduced into the T cells by electroporation (Wiesinger et al. 2019 Cancers (Basel) 11:1198).

In some embodiments, a composition comprising the CAR T cells comprise one or more of helper T cells, cytotoxic T cells, memory T cells, naïve T cells, regulatory T cells, natural killer T cells, or combinations thereof.

### II. CMV Specific T Cells

In some cases, the method includes a step of preparing T cells specific for cytomegalovirus (CMV) and expressing a chimeric antigen receptor (CAR), the method comprising: (a) providing T cells (e.g., PBMC) from a cytomegalovirus CMV seropositive human donor; (b) exposing the PBMC to at least one CMV antigen; (c) treating the exposed cells to produce a population of cells enriched for stimulated cells specific for CMV; (d) transducing at least a portion of the enriched population of cells with a vector expressing a CAR, thereby preparing T cells specific for CMV and expressing a CAR. In various cases: the step of treating the exposed cells (e.g., using a selection step) to produce a population of cells enriched for stimulated cells specific for CMV comprises treating the stimulated cells to produce a population of cells enriched for cells expressing an activation marker (e.g., IFN-γ or IL-13); the PBMC are cultured for less than 5 days (less than 4, 3, 2, 1 days) prior to exposure to the CMV antigen; the cells are exposed to the CMV antigen for fewer than 3 days (fewer than 48 hrs, 36 hrs, 24 hrs) the CMV antigen is pp65 protein or an antigenic portion thereof, the CMV antigen comprises two or more different antigenic CMV pp65 peptides; the step of transducing the enriched population of cells does not comprise CD3 stimulation; the step of transducing the enriched population of cells does not comprise CD28 stimulation; the step of transducing the enriched population of cells does not comprise CD3 stimulation or CD28 stimulation; the enriched population of cells is at least 40% (e.g., 50%, 60%, 70%) IFN-γ positive, at least 20% (e.g., 25%, 30%, 35%) CD8 positive, and at least 20% (e.g., 25%, 30%, 35%) CD4 positive; the enriched population of cells are cultured for fewer than 10 (fewer than 9, 8, 7, 5, 3, 2) days prior to the step of transducing the enriched population of cells with a vector encoding a CAR. In some cases, the T cells are from a CMV positive donor and are exposed to a CMV antigen such as CMV pp65 or a mixture of CMV protein peptides (for example 10-20 amino acid peptides that are fragments of pp65) in the presence of IL-2 to create a population of stimulated cells. In some cases, the population of stimulated cells is treated to prepare a population of cells that express IFN-γ. In some cases, the CMV/CAR T cells do not recognize an antigen from a second virus. For example, they do not recognize an Epstein-Barr virus antigen or an influenza virus antigen or an Adenovirus antigen.

III. Treatment of Patients Infected with HIV

Aspects of the present disclosure provide methods for treating a subject infected with by administering immune cells, e.g., CMV-specific T cells that express an HIV-CAR, and a CMV vaccine.

### (a) Subjects

The subject to be treated by the methods described can be a human subject infected with HIV, including a subject taking antiretroviral therapy (ART). It can be administered to subjects and a viral load above 200 copies/ml or below 200 copies/ml and subjects with an undetectable viral load. Subjects may be being treated with or more of: a nucleoside reverse transcriptase inhibitor (NRTI), a nonnucleoside reverse transcription inhibitors (NNRTI), a protease inhibitor (PI), an entry or fusion inhibitor, and an integrase inhibitor (INSTI). For example, a subject may be being treated with two NRTIs with an INSTI, NNRTI, or PI and, in some cases, ritonavir or cobicistat.

### (b) Administration

An effective amount of a therapy (e.g., CMV-CAR T cells and a CMV vaccine) can be administered to a subject (e.g., a human) in need of the treatment via any suitable route (e.g., administered locally or systemically to a subject). Suitable modes of administration include injection, infusion, instillation, or ingestion. Injection includes, without limitation, intravenous, intramuscular, intra-arterial, intrathecal, intraventricular, intradermal, intraperitoneal, subcutaneous injection, and infusion. The CMV-CAR T cells and the CMV vaccine can be administered at the same time or sequentially.

An effective amount refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of treatment, the nature of concurrent therapy, if any, the specific route of administration and like factors. An effective amount can be administered in one or more administrations, applications or dosages. The compositions described herein (e.g., CMV HIV CAR T cells and CMV vaccine) can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Useful doses of CMV HIV T cells include about 5×10⁶, 10×10⁶, 15×10⁶, 20×10⁶, 25×10⁶, 30×10⁶, 35×10⁶, 40×10⁶, 45×10⁶, 50×10⁶, 55×10⁶, 60×10⁶, 65×10⁶, 70×10⁶, 75×10⁶, 80×10⁶, 85×10⁶, 90×10⁶, 95×10⁶, and 100×10⁶ cells. A health care professional can provide dose escalation or de-escalation to a patient as needed. In some embodiments, a single dose of CMV-HIV CAR T cells is administered to the patient. In some embodiments, a second dose of CMV-HIV CAR T cells is administered to the patient.

In some embodiments, an effective amount of a CMV vaccine comprising at least one CMV antigen or a nucleic acid molecule encoding at least one CMV antigen is administered to the subject. In some embodiments, a CMV vaccine comprising at least one CMV antigen or a nucleic acid molecule encoding at least one CMV antigen is administered in an amount sufficient to stimulate an immune response in the subject.

In some embodiments, a CMV vaccine is administered at the same time the CMV-HIV CAR T cells are administered. In some embodiments, the CMV vaccine is administered before the administration of the CMV-HIV CAR T cells. In some embodiments, the CMV vaccine is administered after the administration of the CMV-HIV CAR T cells. In some embodiments, the CMV vaccine is administered before and after the administration of the CMV-HIV CAR T cells. In some embodiments, the CMV vaccine is administered in single or repeat dosing. In some embodiments, the CMV-HIV CAR T cells are administered in single or repeat dosing.

In some embodiments, the subject is administered the CMV vaccine prior to the administration of the CMV-HIV CAR T cells. In some embodiments, the CMV vaccine is administered one, two, three, four, five, six, seven, eight, nine, or ten days before the administration of the CMV-HIV CAR T cells. In some embodiments, the CMV vaccine is administered about 1, 2, 3, 4, 5, 6, 7, 8, 9, 10, 11, 12, 14, 16, 18, 20, 22, 24, 36, or 48 hours before the administration of the CMV-HIV CAR T cells. In some embodiments, the CMV vaccine is administered about one, two, three, or four weeks before the administration of the CMV-HIV CAR T cells.

In some embodiments, the subject is administered the CMV vaccine following the administration of the CMV-HIV CAR T cells. In some embodiments, the CMV vaccine is administered one, two, three, four, five, six, seven, eight, nine, or ten days after the administration of the CMV-HIV CAR T cells. In some embodiments, the CMV vaccine is administered about 1, 2, 3, 4, 5, 6, 7, 8, 9, 10, 11, 12, 14, 16, 18, 20, 22, 24, 36, or 48 hours after the administration of the CMV-HIV CAR T cells. In some embodiments, the CMV vaccine is administered about one, two, three, or four weeks after the administration of the CMV-HIV CAR T cells. In some embodiments, this can be in addition to administering the CMV vaccine prior to the administration of the CMV-HIV CAR T cells, thus in some embodiments, the subject would be administered at least one CMV vaccine before administration of the CMV-HIV CAR T cells and at least one CMV vaccine after the administration of the CMV-HIV CAR T cells.

In some embodiments, the at least one CMV vaccine is administered prior to or subsequent to administering the CMV-specific T cells in single or repeat dosing.

### (c) CMV Vaccine: CMV Antigen or a Nucleic Acid Encoding a CMV Antigen

A useful CMV vaccine can comprise one or more CMV antigens or one or more nucleic acids encoding one or more CMV antigens. A CMV antigen can be a CMV protein, a fragment of a CMV protein, a modified CMV protein, a fragment of a modified CMV protein, a mutated CMV protein or a fragment thereof, or a fusion CMV protein or a fragment thereof. In some embodiments, a useful CVM vaccine comprises one or more nucleic acids encoding one or more CMV antigens. Examples of CMV antigens include pp65, IE1 exon 4 (IE1/e4), IE2 exon 5 (IE2/e5), fusions thereof, and antigenic fragments thereof, and variants of each thereof with 1, 2, 3, 4, or 5 amino acid modifications. In some embodiments, the variants comprise 1-2 amino acid substitutions or 1-5 amino acid substitutions. In some embodiments, the amino acid substitutions are conservative. Examples of modified CMV protein antigens and fragments thereof may be found in U.S. Pat. No. 7,163,685.

In some embodiments, a CMV antigen comprises a sequence selected from SEQ ID NOs: 57-64 and variants thereof having 1, 2, 3, 4, or 5 amino acid modifications. In some embodiments, the variants comprise 1-2 amino acid substitutions or 1-5 amino acid substitutions. In some embodiments, the amino acid substitutions are conservative. In some embodiments, a CMV antigen can comprise a fragment of any of SEQ ID NOs: 57-64. The fragment can comprise of consist of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 contiguous amino acids of any of SEQ ID NOs: 57-64.

Some examples of nucleic acids that can encode a CMV antigen are DNA, RNA, mRNA, vector, viral vector, lentiviral vector, MVA vector, bacterial artificial chromosome (BAC), vaccinia virus vector, adenovirus vector, adeno-associated virus vector, and others known in the art. Useful nucleic acids can encode one or more CMV antigens. In some embodiments, the nucleotide sequence for a CMV antigen is optimized.

Fusion CMV protein antigens may comprise two or more CMV proteins, modified CMV proteins, mutated CMV proteins or any antigenic fragments thereof. In some embodiments, a useful fusion protein is a fusion of IE1 exon 4 (IE1/e4) and IE2 exon 5 (IE2/e5), IE1/e4-IE2/e5 (IEfusion; e.g., SEQ ID NO: 58). In some embodiments, a useful fusion protein comprises SEQ ID NO:58 or a variant thereof with 1-5 amino acid modifications. In some embodiments, a variant comprises 1-2 amino acid substitutions or 1-5 amino acid substitutions. In some embodiments, the amino acid substitutions are conservative.

A useful CMV vaccine can be a rMVA vaccine comprosing a modified vaccinia Ankara (MVA) vaccine platform in combination with the bacterial artificial chromosome (BAC) technology. Modified Vaccinia Ankara (MVA) is a genetically engineered, highly attenuated strain of vaccinia virus that does not propagate in most mammalian cells. This property minimally impacts viral or foreign gene expression because the ability of MVA to propagate in mammalian cells is blocked at late stage viral assembly. However, the DNA continues to replicate and therefore acts as an efficient template for RNA biosynthesis leading to high levels of protein synthesis. MVA also has a large foreign gene capacity and multiple integration sites, two features that make it a desirable vector for expressing vaccine antigens. MVA has a well-established safety record and versatility for the production of heterologous proteins. In fact, MVA-based vaccines for treatment of infectious disease and cancer have been developed and reached Phase I/II clinical trials. MVA is appealing as a vaccine vector for CMV antigens in individuals who are both severely immunosuppressed and experiencing additional complications such as malignancy or organ failure and needing a transplant.

CMV Triplex Vaccine is a recombinant MVA that expresses three CMV antigens, i.e., at least a portion or Immediate-Early Gene-1 (IE1), at least a portion of Immediate-Early Gene-2 (IE2) and at least a portion of pp65. The IE1 antigen and the IE2 antigen can be expressed a fusion protein, Expression of the CMV antigens can be under the control of a modified H5 (mH5) promoter. A CMV Triplex Vaccine is fully described in U.S. Pat. No. 8,580,276 and in Wang et al. (Vaccine 28:1547, 2010. The CMV Triplex Vaccine can express CMV pp65 and a CMV IE fusion protein (IEfusion). The IEfusion can include an antigenic portion of IE1 (e.g., Exon 4) and an antigenic portion of 1E2 (e.g., Exon 5), wherein the antigenic portions elicit an immune response when expressed by a vaccine.

Various modifications and/or insertion sites can made with the purpose of increasing the stability of Triplex simultaneously expressing IE1, IE2 and pp65 in a single MVA vector (see, e.g., WO 2019/217922).

As explained in U.S. Pat. No. 8,580,276, the CMV Triplex Vaccine includes three of the best recognized antigens in the CD8 subset: pp65, IE1, and IE2. There is no region of homology greater than 5 amino acids between the major exons of both proteins. Individually, both antigens are recognized broadly by almost 70% of the general population.

Any of the vaccine compositions disclosed in U.S. Pat. Nos. 7,163,685, 8,580,276, 9,675,689 US20170246292 A1 may be used for the methods and compositions provided herein and are hereby incorporated by reference in their entirety and for all purposes.

### Selected CMV Antigen Amino Acid Sequences:

CMV pp65 protein
(UniProt ID: P06725; SEQ ID NO: 57)
MESRGRRCPEMISVLGPISGHVLKAVFSRGDTPVLPHETRLLQTGIHVRVSQPSLILVSQYT
PDSTPCHRGDNQLQVQHTYFTGSEVENVSVNVHNPTGRSICPSQEPMSIYVYALPLKMLNI
PSINVHHYPSAAERKHRHLPVADAVIHASGKQMWQARLTVSGLAWTRQQNQWKEPDVYY
TSAFVFPTKDVALRHVVCAHELVCSMENTRATKMQVIGDQYVKVYLESFCEDVPSGKLFMH
VTLGSDVEEDLTMTRNPQPFMRPHERNGFTVLCPKNMIIKPGKISHIMLDVAFTSHEHFGLL
CPKSIPGLSISGNLLMNGQQIFLEVQAIRETVELRQYDPVAALFFFDIDLLLQRGPQYSEHPT
FTSQYRIQGKLEYRHTWDRHDEGAAQGDDDVWTSGSDSDEELVTTERKTPRVTGGGAMA
GASTSAGRKRKSASSATACTSGVMTRGRLKAESTVAPEEDTDEDSDNEIHNPAVFTWPPW
QAGILARNLVPMVATVQGQNLKYQEFFWDANDIYRIFÅELEGVWQPAAQPKRRRHRQDAL
PGPCIASTPKKHRG
IEfusion sequence;
(IE1-IE2; SEQ ID NO: 58)
MVKQIKVRVDMVRHRIKEHMLKKYTQTEEKFTGAFNMMGGCLQNALDILDKVHEPFEE
MKCIGLTMQSMYENYIVPEDKREMWMACIKELHDVSKGAANKLGGALQAKARAKKDE
LRRKMMYMCYRNIEFFTKNSAFPKTTNGCSQAMAALQNLPQCSPDEIMAYAQKIFKIL
DEERDKVLTHIDHIFMDILTTCVETMONEYKVTSDACMMTMYGGISLLSEFCRVLCCYV
LEETSVMLAKRPLITKPEVISVMKRRIEEICMKVFAQYILGADPLRVCSPSVDDLRAIAEE
SDEEEAIVAYTLATAGVSSSDSLVSPPESPVPATIPLSSVIVAENSDQEESEQSDEEEEE
GAQEEREDTVSVKSEPVSEIEEVAPEEEEDGAEEPTASGGKSTHPMVTRSKADQGDIL
AQAVNHAGIDSSSTGPTLTTHSCSVSSAPLNKPTPTSVAVTNTPLPGASATPELSPRKK
PRKTTRPEKVIIKPPVPPAPIMLPLIKQEDIKPEPDFTIQYRNKIIDTAGCIVISDSEEEQGE
EVETRGATASSPSTGSGTPRVTSPTHPLSQMNHPPLPDPLGRPDEDSSSSSSSSCSS
ASDSESESEEMKCSSGGGASVTSSHHGRGGFGGAASSSLLSCGHQSSGGASTGPR
KKKSKRISELDNEKVRNIMKDKNTPFCTPNVQTRRGRVKIDEVSRMERNTNRSLEYKN
LPFTIPSMHQVLDEAIKACKTMQVNNKGIQIIYTRNHEVKSEVDAVRCRLGTMCNLALS
TPFLMEHTMPVTHPPEVAQRTADACNEGVKAAWSLKELHTHQLCPRSSDYRNMIIHA
ATPVDLLGALNLCLPLMQKFPKQVMVRIFSTNQGGFMLPIYETAAKAYAVGQFEQPTE
TPPEDLDTLSLAIEAAIQDLRNKSQ
IE1 sequence (SEQ ID NO: 59):
MVKQIKVRVDMVRHRIKEHMLKKYTQTEEKFTGAFNMMGGCLQNALDILDKVHEPFEE
MKCIGLTMQSMYENYIVPEDKREMWMACIKELHDVSKGAANKLGGALQAKARAKKDE
LRRKMMYMCYRNIEFFTKNSAFPKTTNGCSQAMAALQNLPQCSPDEIMAYAQKIFKIL
DEERDKVLTHIDHIFMDILTTCVETMONEYKVTSDACMMTMYGGISLLSEFORVLCCYV
LEETSVMLAKRPLITKPEVISVMKRRIEEICMKVFAQYILGADPLRVCSPSVDDLRAIAEE
SDEEEAIVAYTLATAGVSSSDSLVSPPESPVPATIPLSSVIVAENSDQEESEQSDEEEEE
GAQEEREDTVSVKSEPVSEIEEVAPEEEEDGAEEPTASGGKSTHPMVTRSKADQ
IE2 sequence (SEQ ID NO: 61):
MGDILAQAVNHAGIDSSSTGPTLTTHSCSVSSAPLNKPTPTSVAVTNTPLPGASATPEL
SPRKKPRKTTRPFKVIIKPPVPPAPIMLPLIKQEDIKPEPDFTIQYRNKIIDTAGCIVISDSE
EEQGEEVETRGATASSPSTGSGTPRVTSPTHPLSQMNHPPLPDPLGRPDEDSSSSSS
SSCSSASDSESESEEMKCSSGGGASVTSSHHGRGGFGGAASSSLLSCGHQSSGGAS
TGPRKKKSKRISELDNEKVRNIMKDKNTPFCTPNVQTRRGRVKIDEVSRMFRNTNRSL
EYKNLPFTIPSMHQVLDEAIKACKTMQVNNKGIQIIYTRNHEVKSEVDAVRCRLGTMCN
LALSTPFLMEHTMPVTHPPEVAQRTADACNEGVKAAWSLKELHTHQLCPRSSDYRNM
IIHAATPVDLLGALNLCLPLMQKFPKQVMVRIFSTNQGGFMLPIYETAAKAYAVGQFEQ
PTETPPEDLDTLSLAIEAAIQDLRNKSQ
IE2 H363A sequence (SEQ ID NO: 62):
MGDILAQAVNHAGIDSSSTGPTLTTHSCSVSSAPLNKPTPTSVAVTNTPLPGASATPEL
SPRKKPRKTTRPFKVIIKPPVPPAPIMLPLIKQEDIKPEPDFTIQYRNKIIDTAGCIVISDSE
EEQGEEVETRGATASSPSTGSGTPRVTSPTHPLSQMNHPPLPDPLGRPDEDSSSSSS
SSCSSASDSESESEEMKCSSGGGASVTSSHHGRGGFGGAASSSLLSCGHQSSGGAS
TGPRKKKSKRISELDNEKVRNIMKDKNTPFCTPNVQTRRGRVKIDEVSRMFRNTNRSL
EYKNLPFTIPSMHQVLDEAIKACKTMQVNNKGIQIIYTRNHEVKSEVDAVRCRLGTMON
LALSTPFLMEATMPVTHPPEVAQRTADACNEGVKAAWSLKELHTHQLCPRSSDYRNM
IHAATPVDLLGALNLCLPLMQKFPKQVMVRIFSTNQGGFMLPIYETAAKAYAVGQFEQ
PTETPPEDLDTLSLAIEAAIQDLRNKSQ
IE2 H369A sequence (SEQ ID NO: 63):
MGDILAQAVNHAGIDSSSTGPTLTTHSCSVSSAPLNKPTPTSVAVTNTPLPGASATPEL
SPRKKPRKTTRPFKVIIKPPVPPAPIMLPLIKQEDIKPEPDFTIQYRNKIIDTAGCIVISDSE
EEQGEEVETRGATASSPSTGSGTPRVTSPTHPLSQMNHPPLPDPLGRPDEDSSSSSS
SSCSSASDSESESEEMKCSSGGGASVTSSHHGRGGFGGAASSSLLSCGHQSSGGAS
TGPRKKKSKRISELDNEKVRNIMKDKNTPFCTPNVQTRRGRVKIDEVSRMFRNTNRSL
EYKNLPFTIPSMHQVLDEAIKACKTMQVNNKGIQIIYTRNHEVKSEVDAVRCRLGTMCN
LALSTPFLMEHTMPVTAPPEVAQRTADACNEGVKAAWSLKELHTHQLCPRSSDYRNM
IHAATPVDLLGALNLCLPLMQKFPKQVMVRIFSTNQGGFMLPIYETAAKAYAVGQFEQ
PTETPPEDLDTLSLAIEAAIQDLRNKSQ
IE2 H363A/H369A sequence (SEQ ID NO: 64):
MGDILAQAVNHAGIDSSSTGPTLTTHSCSVSSAPLNKPTPTSVAVTNTPLPGASATPEL
SPRKKPRKTTRPFKVIIKPPVPPAPIMLPLIKQEDIKPEPDFTIQYRNKIIDTAGCIVISDSE
EEQGEEVETRGATASSPSTGSGTPRVTSPTHPLSQMNHPPLPDPLGRPDEDSSSSSS
SSCSSASDSESESEEMKCSSGGGASVTSSHHGRGGFGGAASSSLLSCGHQSSGGAS
TGPRKKKSKRISELDNEKVRNIMKDKNTPFCTPNVQTRRGRVKIDEVSRMFRNTNRSL
EYKNLPFTIPSMHQVLDEAIKACKTMQVNNKGIQIIYTRNHEVKSEVDAVRCRLGTMCN
LALSTPFLMEATMPVTAPPEVAQRTADACNEGVKAAWSLKELHTHQLCPRSSDYRNM
HIHAATPVDLLGALNLCLPLMQKFPKQVMVRIFSTNQGGFMLPIYETAAKAYAVGQFEQ
PTETPPEDLDTLSLAIEAAIQDLRNKSQ

The invention is further described in the following examples, which do not limit the scope of the invention described in the claims.

### EXAMPLES

Described below, inter alia, is the design and preparation of CMV-HIV CAR T cells using cells obtained from patients on ART as well as studies using such cells and a CMV vaccine in a murine model of HIV infection.

### Example 1: N6-CAR T Cells Exhibit Potent Effector Functions in Vitro

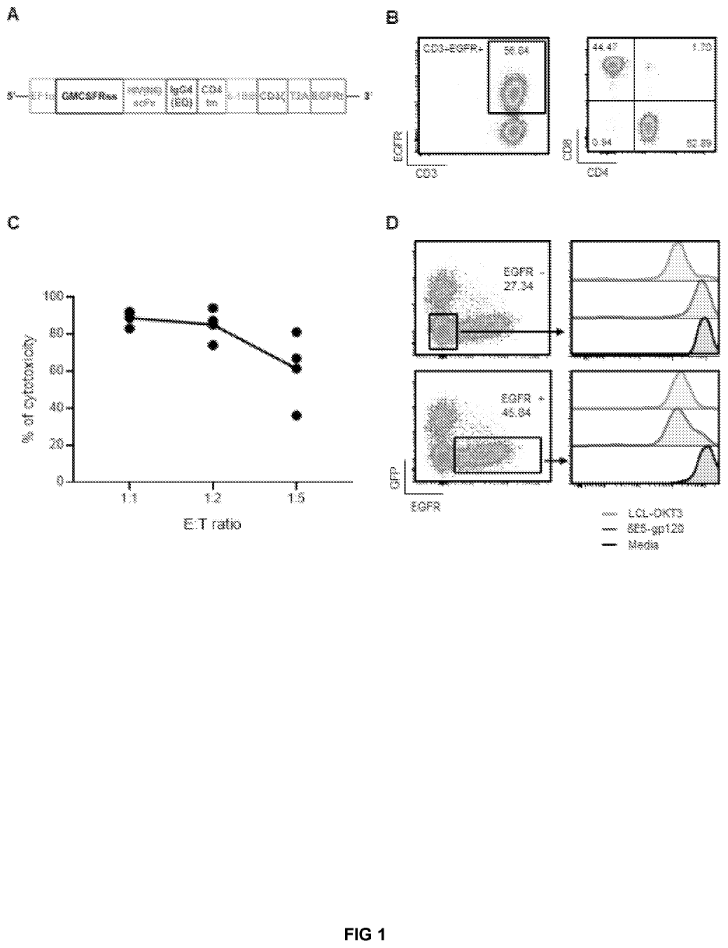

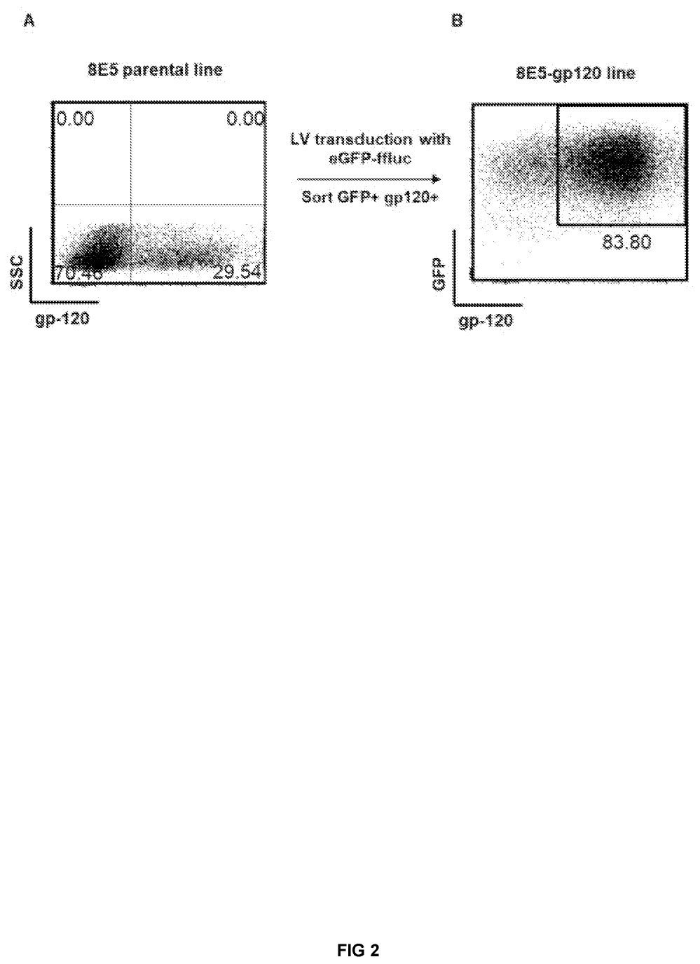

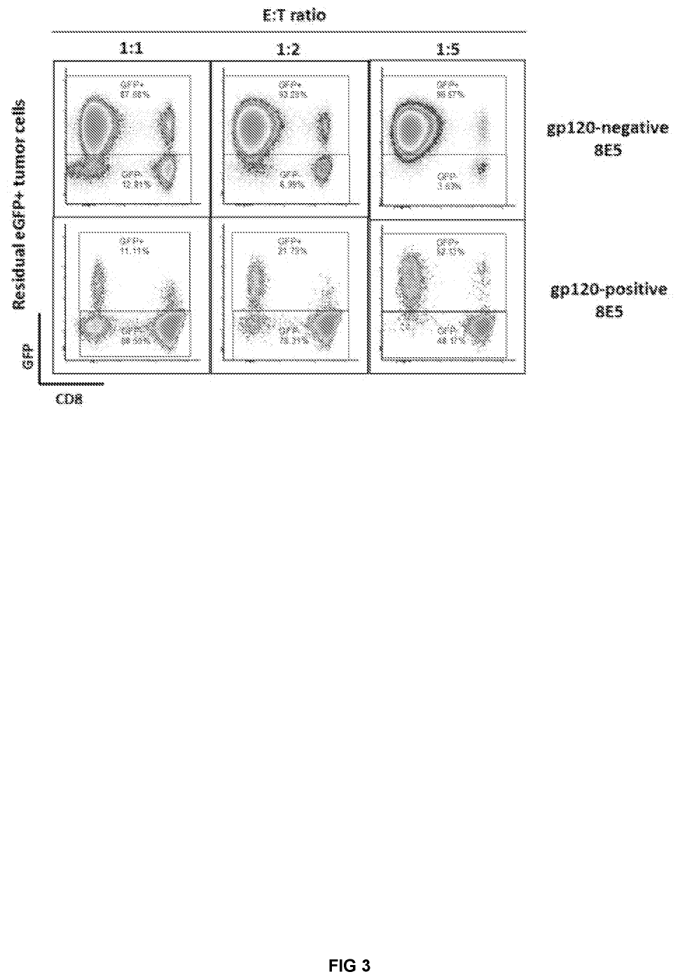

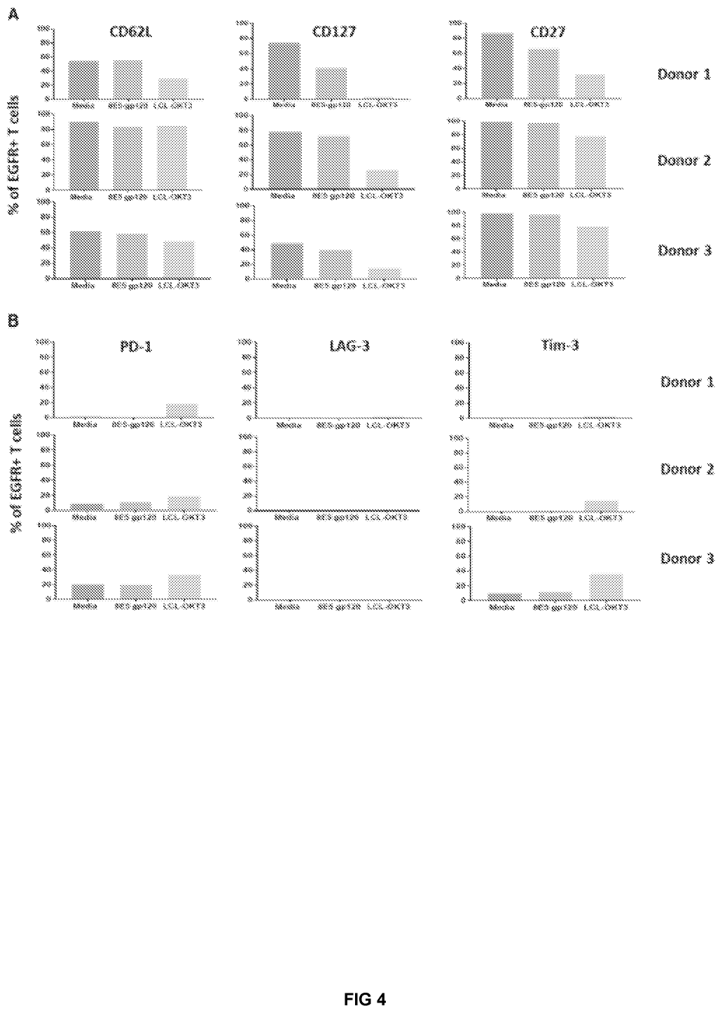

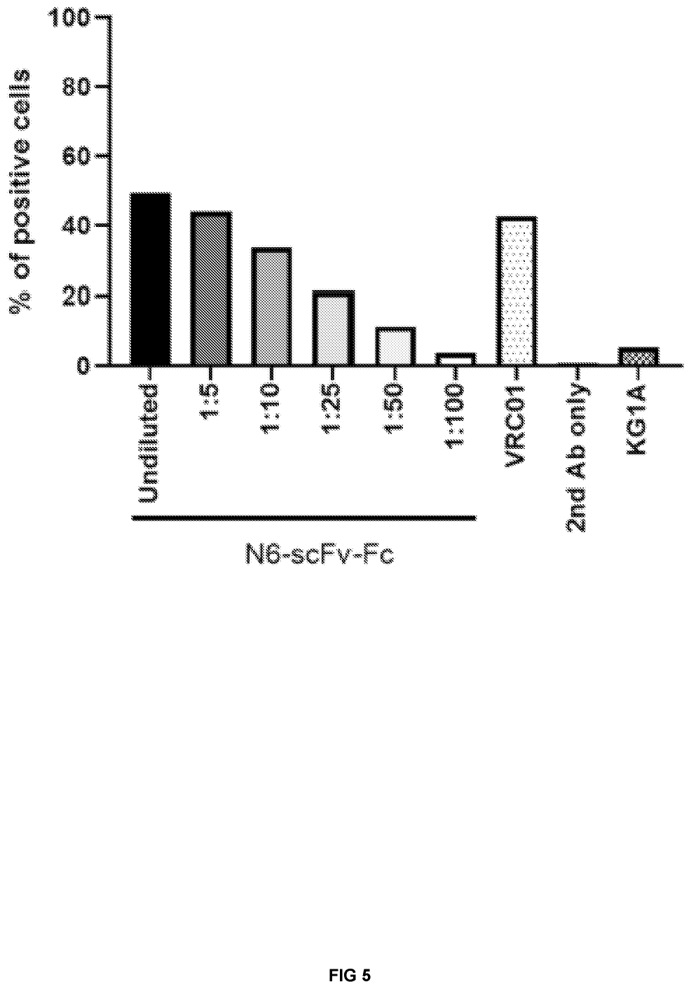

A N6-based CAR T cell product was prepared by transducing primary T cells isolated from HIV^{neg} donors with a lentiviral vector (LV) encoding a CAR containing the scFv of the bNAb N6 (FIG. 1Error! Reference source not found.A). The CAR includes a variant IgG4 spacer between the scFv ectodomain and the transmembrane domain²⁴, CD4 transmembrane domain, a 4-1BB co-stimulatory domain²⁵⁻²⁹ and a CD3zeta domain. A truncated human epidermal growth factor receptor (EGFRt) was added to the CAR construct to serve as an element for immunomagnetic purification, cell tracking by flow cytometry and immunohistochemistry, and potential in vivo cell ablation with the anti-EGFR antibody cetuximab.³⁰ After ˜15-days of in vitro expansion, the final T cell products contained on average 38.16%±8.87% EGFR⁺ CAR T cells, of which 69.63%±16.63% were CD4⁺ and 31.57%±17.76% were CD8⁺ T cells (mean±SD, n=4, FIG. 1B shows representative FACS plots). We examined whether N6-CAR T cells elicited cytotoxic function by performing a 96-hour killing assay that targeted 8E5-gp120 cells. These 8E5-gp120 cells were obtained by engineering 8E5 cells to express eGFP-ffLuc and then sorting for co-expression of eGFP and surface gp120 (FIG. 2). N6-CAR T cell products and 8E5-gp120 cells were cocultured at various effector-to-target (E:T) ratios. Flow cytometric analysis of the remaining target cells demonstrated that N6-CAR T cells, normalized to mock T cells, efficiently killed 8E5-gp120 cells (FIG. 1C). In a separate experiment, N6-CAR T cells were co-cultured at various E:T ratios with purified gp120-negative or gp120-positive 8E5 cells. Efficient and gp120-specific killing was observed against gp120-positive 8E5 cells, but not against the gp120-negative 8E5 cells (FIG. 3). Finally, only gated EGFR⁺ CAR T cells from a mixed T cell population, but not the gated CAR-negative T cell fraction, exhibited proliferative capacity after stimulation with 8E5-gp120 cells (FIG. 1D). Flow cytometric analysis revealed that these stimulated N6-CAR T cells maintained sustained memory (CD62L=66.07%, CD127=51.42% and CD27=87.02%, average of three donors), and low exhaustion features (programmed cell death-1 [PD-1]=10.84%, lymphocyte-activation gene-3 [LAG-3]=0.26%, and T cell immunoglobulin and mucin domain-3 [Tim-3]=4.71%, average of three donors) (FIG. 4).

### Example: N6 scFv-Fc does not Cross-React with Normal Human Tissues

To assess the potential off-target effects of N6-CARs, immunostaining with soluble N6 scFv-Fc was performed on normal human tissues. As expected, a concentration-dependent immunostaining was observed using either the N6 scFv-Fc or a positive control (the anti-gp120 bNAb VRC01 obtained from the NIH HIV Reagent Program) on 8E5-gp120 cells, but not on gp120-negative leukemic KG-1a cells (FIG. 5). Then, N6 scFv-Fc was used for pan-immunostaining on 37 frozen human tissues from three unrelated normal donors [Charles River Labs, CRL study no: 20182940]. The immunopathological analysis did not reveal membrane signals on these tissues. However, cytoplasmic staining was observed in epithelial cells in the esophagus (mucosa), kidney (renal pelvis), pituitary (adenohypophysis), salivary gland (ducts), skin (sweat glands), thymus (epithelial-reticular), and ureter (mucosa) and in the colloid in the thyroid. The binding to cytoplasmic sites is considered of little to no toxicologic significance due to the limited ability of antibody-based therapeutics to access the cytoplasmic compartment in vivo.³¹. Overall, the immunohistochemistry staining analysis supports the low risk of clinically relevant off-target tissue cross-reactivity of N6-CAR.

### Example 3: CMV-HIV CAR T Cells can be Manufactured at Clinical Scale from HIVneg and HIVpos Donors

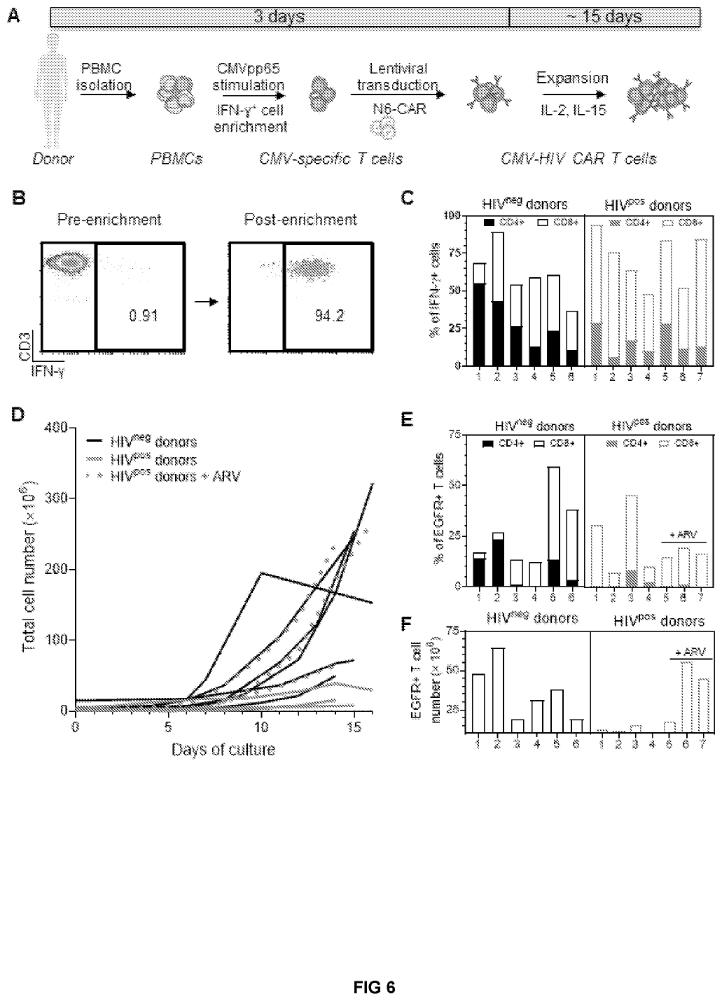

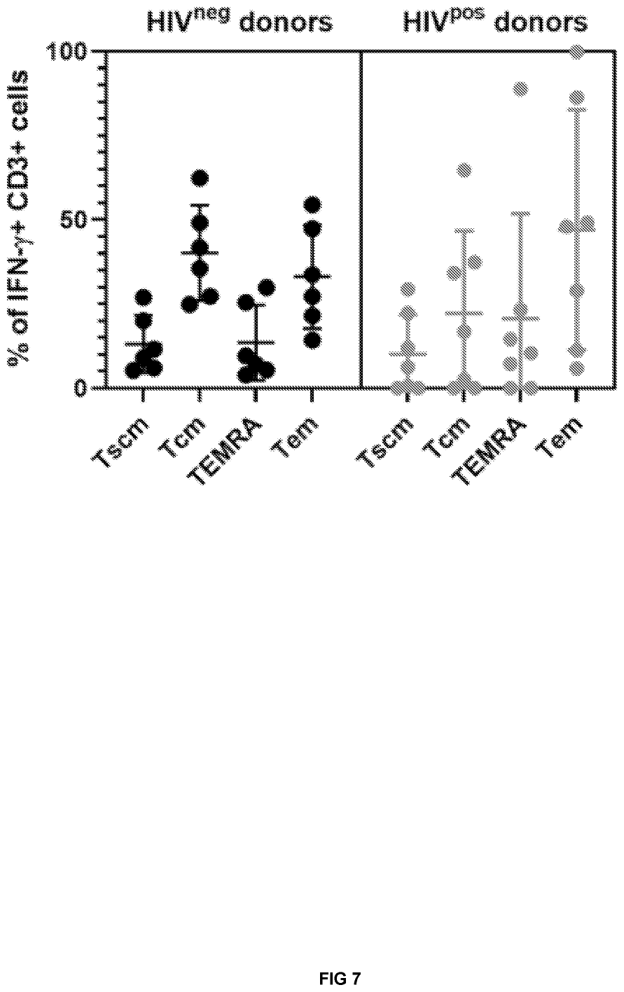

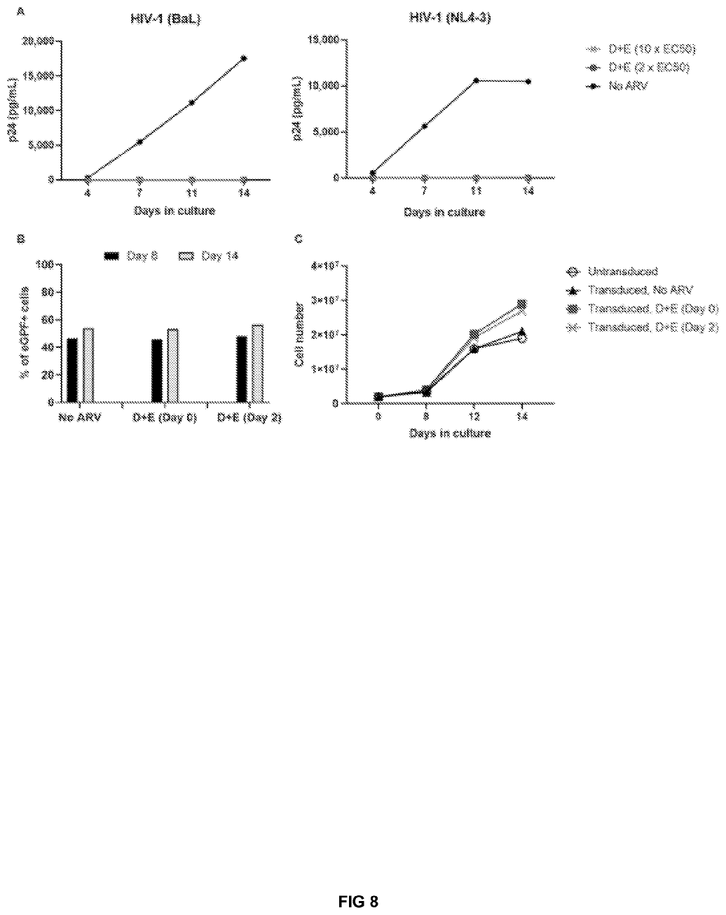

CMV-specific T cells were isolated using a GMP-compliant CliniMACS Prodigy® automated closed system as previously described^{33, 34} and transduced with lentiviral vector encoding N6-CAR (FIG. 2A). Briefly, PBMCs were collected from CMVpos HIVneg or HIVpos donors on ART (Table 1) and processed in the CliniMACS Prodigy® Cytokine Capture System (CCS) by stimulation with a PepTivator® overlapping CMVpp65 peptide pool, followed by labeling with Catchmatrix reagent and anti-IFN-γ microbeads. IFN-γ+ cells were then isolated via magnetic selection in the CliniMACS Prodigy® system (FIG. 6B shows representative FACS plots). CMV-specific T cells (IFN-γ+CD3+) from HIVneg donors were enriched from 4.33%±3.46% to 74.71%±9.17% of the total viable T cells (mean±SD, n=6, FIG. 6C). Similarly, IFN-γ+ T cells from HIVpos donors were enriched from 1.91%±0.94% to 67.28%±17.04% of the total viable T cells (mean±SD, n=7, FIG. 2C). Unlike CMV-specific T cells isolated from HIVneg donors that had similar proportions of CD4+ and CD8+ T cells (42.34%±20.30% and 46.18%±18.86%, respectively), CMV-specific T cells from HIVpos donors had a higher content in CD8+ cells (78.04%±9.21%) as compared to CD4+ cells (25.59%±12.19%) (mean±SD, FIG. 6C). This observation is consistent with previous reports showing that PLWH have a higher proportion of CD8+ CMV-specific T cells as compared to HIVneg individuals.35, 36 Of note, the overall composition in memory T cell subsets was similar between CMV-specific T cells isolated from HIVneg and HIVpos donors (FIG. S5). Recovered IFN-γ+ T cells (˜1×10⁶) were then transduced with the N6-CAR lentiviral vector at MOI 3 to generate CMV-HIV CAR T cells and expanded in the presence of IL-2 (50 U/mL) and IL-15 (1 ng/mL) for ˜15 days. To assess the effect of endogenous reactivation of HIV, we first expanded the CAR T cells in the absence of antiretroviral drugs (ARV). At the end of the culture, the total cell number was 209.5×10⁶ t 97.62×10⁶ for HIVneg donors and 13.49×10⁶±12.17×10⁶ for HIVpos donors (mean±SD, FIG. 6D). ARV (43 nM darunavir and 279 nM enfuvirtide) were supplemented to the medium during the CAR T cell expansion of three HIVpos donors to inhibit HIV replication. This cocktail was shown to prevent HIV replication in vitro (FIG. 8A), without affecting lentiviral transduction efficiency (FIG. 88) and cell expansion (FIG. 8C). Interestingly, the three best cell expansions from HIVpos donors (64.1×10⁶, 237.2×10⁶ and 269.53×10⁸) occurred in the presence of ARV (FIG. 6D, red dotted lines). Thus, with ARV, HIVpos donor cells can expand as well as HIVneg donor cells. To further explore the difference between products made from HIVneg and HIVpos donors, transduction efficiency was assessed by measuring EGFR expression with flow cytometry in the final cell products. Similar EGFR expression levels were observed in cell products derived from HIVneg (24.68%±18.34%) and HIVpos donors (21.45%±12.65%) (mean±SD, FIG. 96E). As expected, CMV-HIV CAR T cells from HIVpos donors consisted of a higher percentage of CD8+ cells (86.46%±16.52%) as compared to CD4+ cells (10.49%±6.62%), whereas the proportion of CD8+ and CD4+ cells within CMV-HIV CAR T cells manufactured from HIVneg donors was 61.35%±40.63% and 43.07%±42.56%, respectively (mean±SD, FIG. 6E). Finally, we assessed the final number of CAR T cells manufactured per campaign (FIG. 6F). The average CAR T cell number in HIVpos-derived cell product expanded in presence of ARV was 34.19×10⁶.

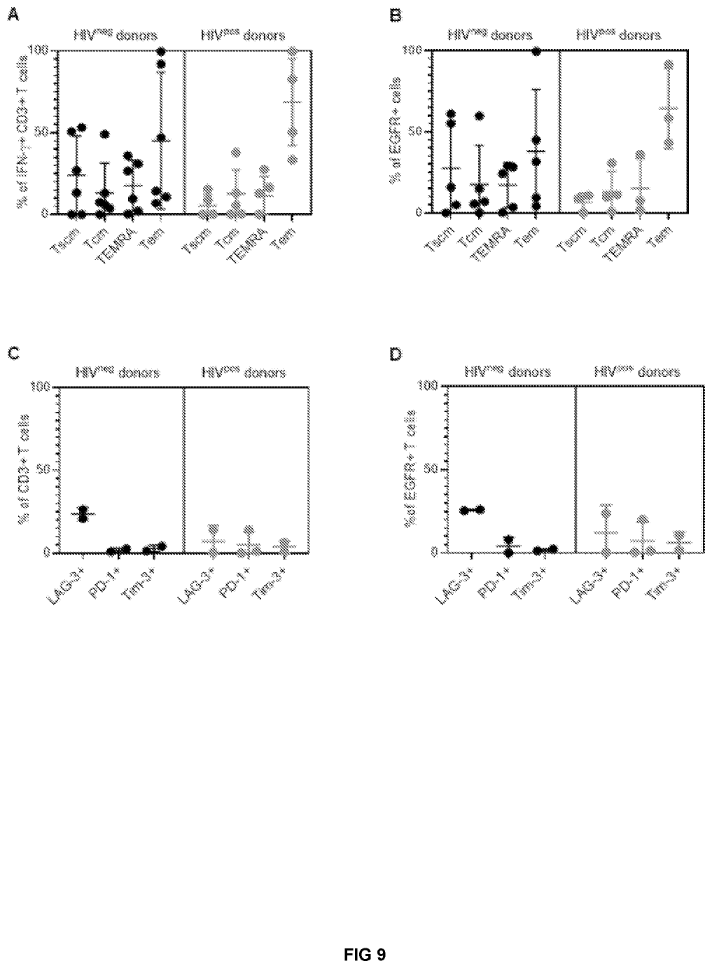

Analysis of T cell memory subsets in the final T cell products showed that HIVneg donor-derived cell products had 19.03%±24.52% CD27+CD45RA+ stem cell memory T cells (Tscm), 18.92%±27.40% CD27+CD45RA− central memory (Tcm), 18.78%±22.41% CD27-CD45RA+ effector memory RA (TEMRA), and 43.26%±37.37% CD27-CD45RA− effector memory T cells (Tem) (mean±SD, FIG. 9A). In comparison, HIVpos donors-derived cell products contained less Tscm (1.71%±2.39%) than Tem (65.02%±21.76%). Similar observations were made when looking at the cell memory subset composition within the EGFR+ CAR T cells (FIG. 9B). Finally, only low expression levels of the exhaustion markers LAG-3, PD-1 and Tim-3 were observed in the final T cell products (FIG. 9C) or in EGFR+ CAR T cells derived from either HIVpos or HIVneg donors (FIG. 9D).

TABLE 4
HIV^{pos} donor information

| Donor ID | Gender | Age | Ethnicity | Race | ART Regimen |
|---|---|---|---|---|---|
| HIV#551 | N/A | N/A | N/A | N/A | N/A |
| HIV#552 | Male | 52 | Non- | White | Genvoya ® (elvitegravir, cobicistat, |
| | | | Hispanic | | emtricitabine, and tenofovir alafenamide) |
| HIV#553 | Female | 54 | Non- | African | Genvoya ® (elvitegravir, cobicistat, |
| | | | Hispanic | American | emtricitabine, and tenofovir alafenamide) |
| HIV#572 | Female | 50 | Hispanic | White | Biktarvy ® (bictegravir, emtricitabine |
| | | | | | and tenofovir alafenamide) |
| HIV#573 | Male | 54 | Non- | White | Atripla ® (efavirenz, emtricitabine, |
| | | | Hispanic | | and tenofovir) |
| IEQR#2 | Male | 54 | Non- | Caucasian | Descovy, Sustiva |
| | | | Hispanic | | |
| IEQR#3 | Male | 54 | Non- | Caucasian | Descovy, Sustiva |
| | | | Hispanic | | |

N/A: information not available

### Example 4: CMV-HIV CAR T Cells Exhibit HIV and CMV Antigen-Specific Effector Functions

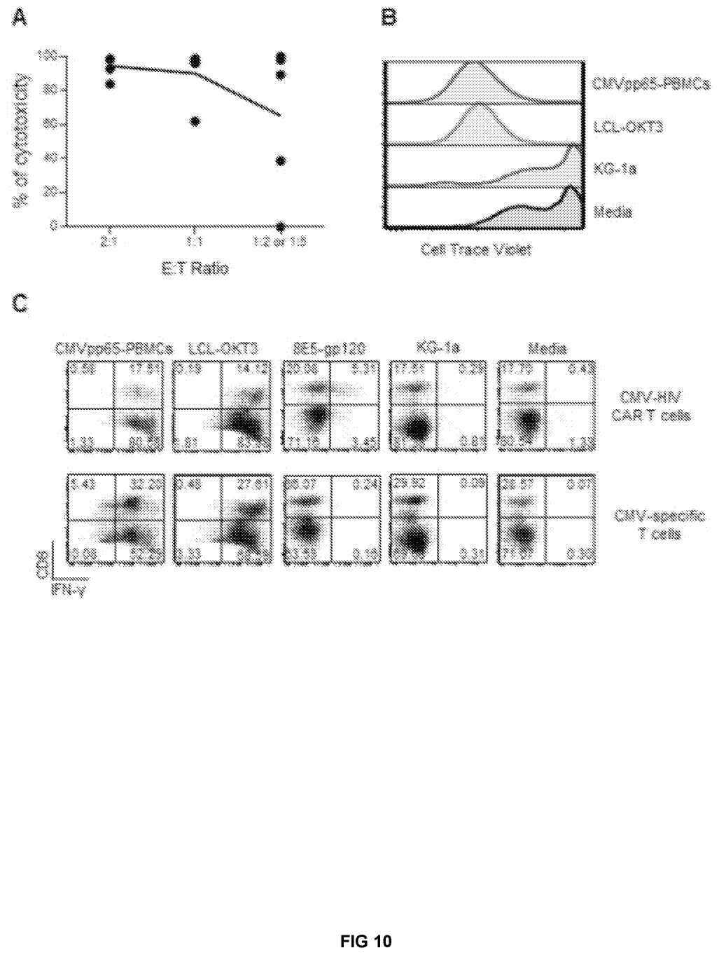

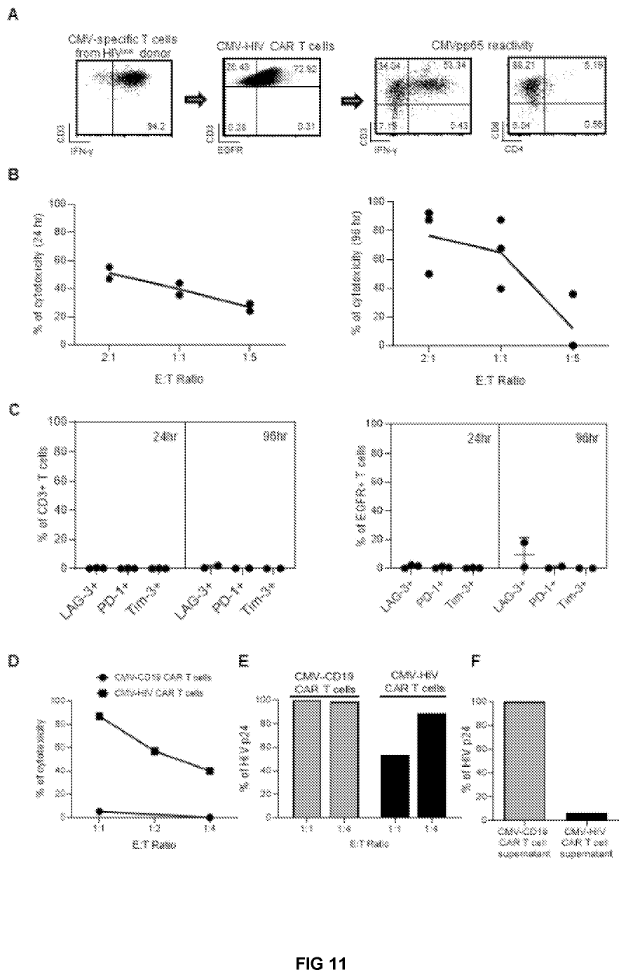

We first showed that CMV-HIV CAR T cells from HIV^{neg} donors were specifically cytotoxic against gp120-expressing cells by performing a 96-hours long-term killing assay using 8E5-gp120 cells as target cells (FIG. 10A). We then evaluated if the CMV-HIV CAR T cells were reactive to CMV antigen stimulation via signaling of their endogenous CMV-specific T cell receptors (TCR). A proliferation assay by CTV dye dilution showed that CMV-HIV CAR T cells proliferate only when co-cultured with CMVpp65 peptide-pulsed autologous PBMCs (CMVpp65-PBMCs) as antigen presenting cells (APCs), or with LCL-OKT3 cells that engage all the TCRs, but not when exposed to KG-1a cells or media (FIG. 10B). Accordingly, higher IFN-γ expression was measured in CMV-HIV CAR T cells after overnight stimulation with either LCL-OKT3, CMVpp65-PBMCs or 8E5-gp120 expressing cells, as compared to stimulation with KG-1a cells or media (FIG. 10C). As expected, CMV-specific T cells only expressed IFN-γ after stimulation with LCL-OKT3 cells and CMVpp65-PBMCs, but not with 8E5-gp120 cells, KG-1a cells or media (FIG. 10C). The relatively low IFN-γ expression in CMV-HIV CAR T cell products after overnight stimulation with 8E5-gp120 suggests that the CAR T cells are slowly killing their target cells.

Similarly, we assessed if CMV-HIV CAR T cells derived from HIV^{pos} donors maintained their effector functions. CMV-HIV CAR T cell products were predominantly CD8⁺ and were reactive to CMVpp65 antigen stimulation, as shown by their high IFN-γ expression after overnight stimulation with CMVpp65-PBMCs (FIG. 11A). In addition, we observed dose-dependent cytotoxicity (FIG. 11B) against 8E5-gp120 cells after both short-term (24 hr, left panel) and long-term (96 hr, right panel) co-cultures. We observed better cytotoxicity with 96-hr co-culture than 24 hr, supporting the optimal killing kinetics with 96 hr in the context of HIV CAR and gp120 target. After 24 h and 96 h co-culture, T cell products and CAR T cells had similar low exhaustion phenotype (FIG. 11C), was similar to its final product indicating our CMV-HIV CAR T cells remain functional and potent after target engagement (FIGS. 9C and 9D). Cytotoxicity of the final cell product against HIV infected cells was further assessed by co-culturing for 7 days CMV-HIV CAR T cells or CMV-CD19 CAR T cells derived from the same donor, with HIV_{N}0.3-infected eGFP⁺ Jurkat cells at various E:T ratios (FIG. 11D).

Compared to CMV-CD19 CAR T cells, CMV-HIV CAR T cells were cytotoxic against HIV-infected cells. In the same experiment, HIV-1 p24 levels were measured by ELISA in the cell supernatants and showed a decrease in p24 release in the presence of CMV-HIV CAR T cells, as compared to CMV-CD19 CAR T cells (FIG. 11E). Finally, higher levels of p24 were detected in the supernatant of HIV^{pos} donor-derived CMV-CD19 CAR T cells as compared to CMV-HIV CAR T cells derived from the same donor (FIG. 11F). Since the only source of HIV in these cultures is from the HIV^{pos} donor, who was aviremic at the time of blood collection, this result suggests that the therapeutic product can eliminate detectable HIV after endogenous reactivation. We next tested whether CMV-HIV CAR T cells have the potential to control HIV viremia and expand in vivo in response to CMVpp65 vaccine in a humanized mouse model of HIV.

### Example 5: CMV-HIV CAR T Cells Exhibit Anti-HIV Activity in a Humanized Mouse Model of HIV

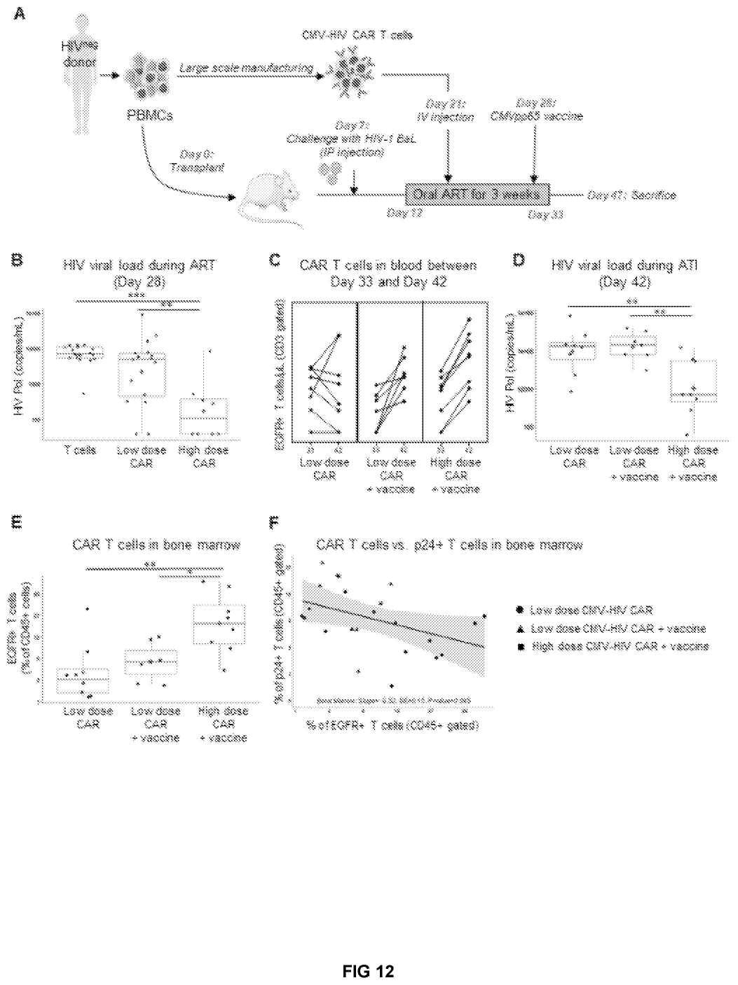

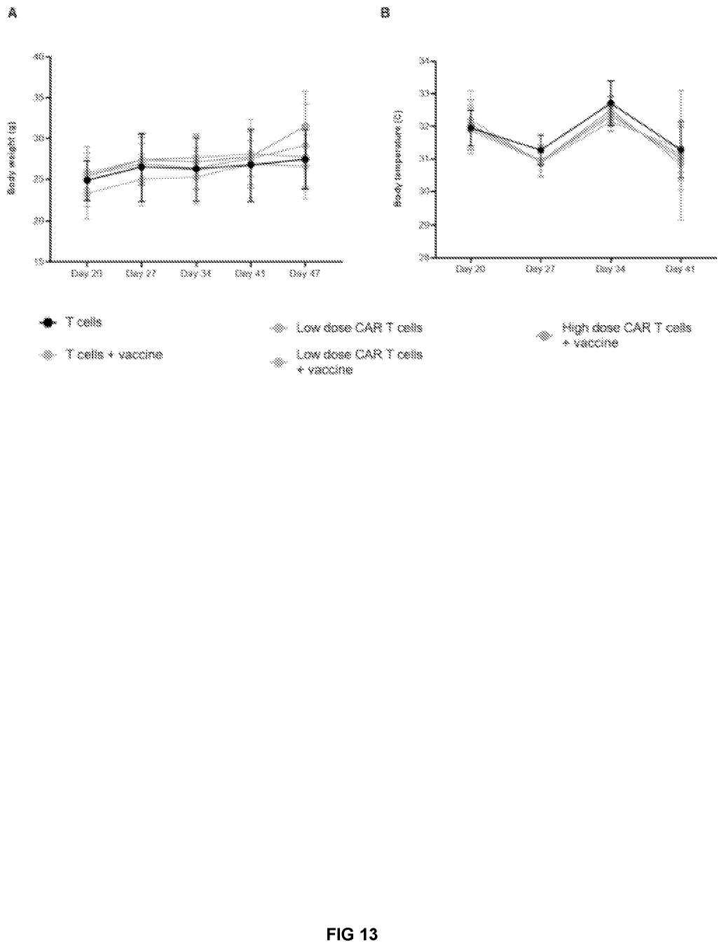

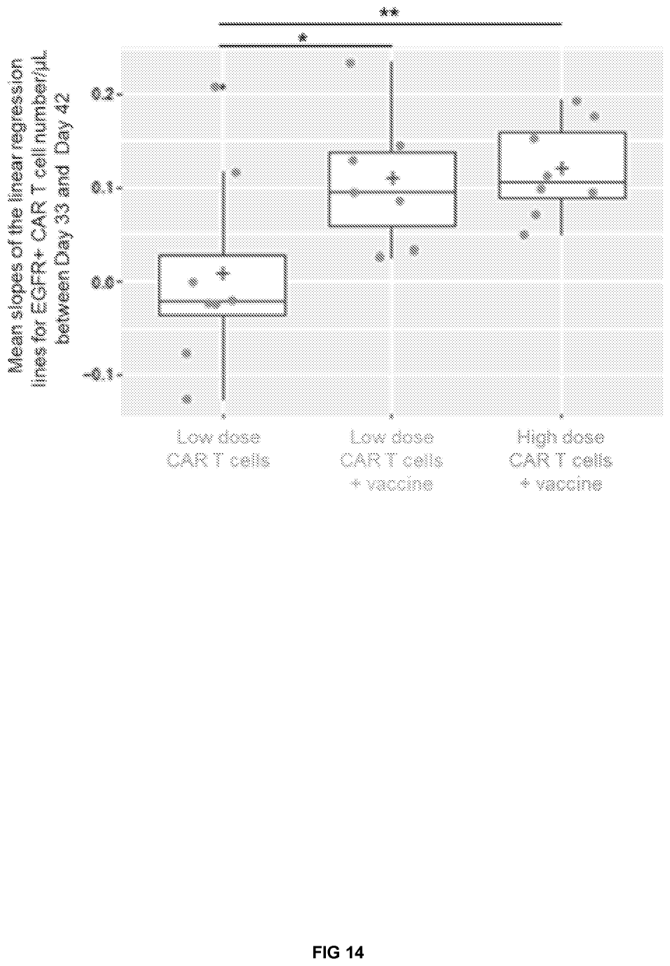

HIV donors were used to generate high numbers of CMV-HIV CAR T cells. The HIV-infected NSG humanized-PBMC (NSG hu-PBMC) mouse model, summarized in FIG. 12A was established by transplant with autologous PBMCs in 3-5-week-old NSG mice (Day 0). On Day 7, mice were challenged with HIV-1 BaL via intraperitoneal (IP) injection, and on Day 12 initiated on a 3-week-long oral ART regimen (Emtricitabine, Tenofovir, Raltegravir) which reduced the plasma viral load from low to undetectable levels. On Day 21, during ART, two cohorts were treated with a single infusion of low dose CMV-HIV CAR T cells (0.1×10⁶ EGFR+ T cells), and either with or without CMVpp65 immunization on Day 28. A group of mice was treated with high dose CMV-HIV CAR T cells (1×10⁸ EGFR+ T cells) followed by CMVpp65 vaccine on Day 28. Two control cohorts included mice treated with CMV-negative T cells (1×10⁶ cells) from the same HIV^{neg} donor, either with or without CMVpp65 vaccine on Day 28. CMV-HIV CAR T cells were well tolerated in all the mice, and no differences in body weight and temperature were observed between the groups (FIG. 13). At Day 28, when mice were on ART prior to vaccination, high-dose of CMV-HIV CAR T cells was found to significantly control HIV plasma viremia compared to control T cell- and to low dose CAR T cell-treated mice (FIG. 12B). EGFR⁺ CAR T cells were then measured in the peripheral blood (FIG. 12C), and for each mouse, the slope of the linear regression lines for EGFR⁺ CAR T cell number/μL between Day 33 (i.e. the final day of ART) and Day 42 (i.e. 9 days after ART interruption) transformed to a log₁₀ scale was calculated (FIG. 14). The mean slope, which represents the rate of expansion of CAR T cells after interruption of ART, was significantly higher for the two vaccinated CAR T cell-treated groups, compared to the unvaccinated low dose CAR T cell-treated cohort. This suggests that the vaccine induced CAR T cell expansion even after ART interruption. At Day 42, after ART interruption and viral rebound, mice that received both a high dose of CMV-HIV CAR T cells and the CMVpp65 vaccine were the only cohort with controlled plasma viremia compared to vaccinated or unvaccinated low dose CAR T cell-treated mice (FIG. 12D). This suggests the importance of CAR T cell dose such that the CMVpp65 vaccine-driven expansion of low dose CAR T cells was not sufficient to reach a therapeutic effect when viremia was high. CAR T cells were also detected in the bone marrow of mice at sacrifice (FIG. 12E), and, notably, an inverse relationship (P-value=0.045) was observed in the frequency of T cells with active HIV-1 infection (i.e., p24⁺ T cells) versus frequency of EGFR+ CAR T cells in the bone marrow, suggesting a CAR-mediated reduction of HIV-infected cells (FIG. 12F).

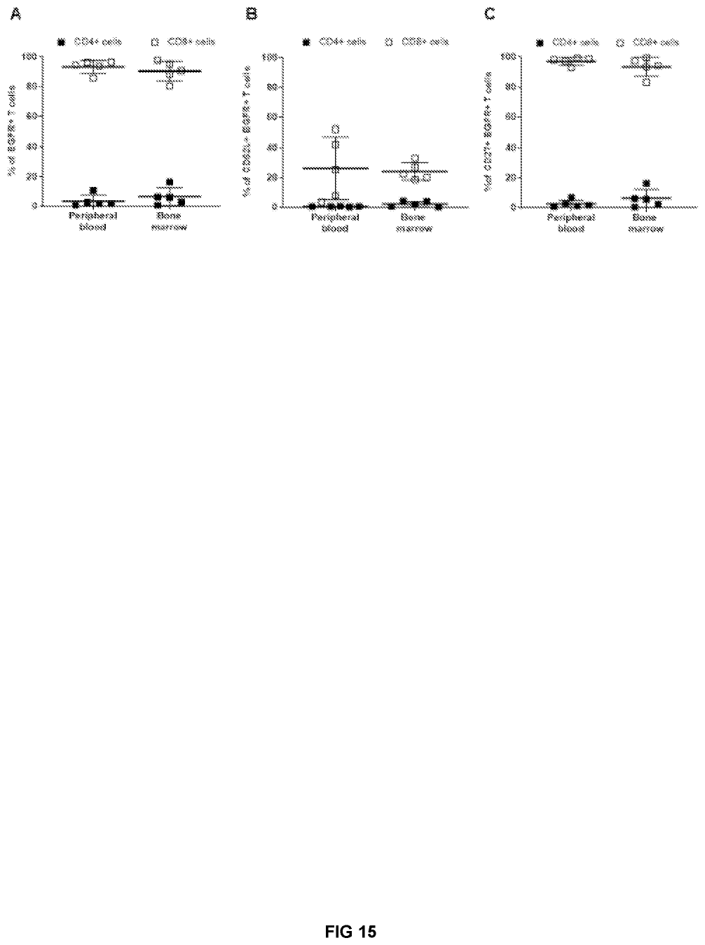

Finally, in a separate experiment, we assessed whether CMV-HIV CAR T cells derived from an HIV^{pos} donor could migrate to the bone marrow, as memory T cells from the bone marrow are long-lasting and persist long after the dissipation of circulating antigen-specific memory T cells.³⁷ EGFR+ CAR T cells (50×10³) were infused into hu-PBMC-NSG mice 14 days after engraftment of HIV-challenged PBMCs (Day 0) and in absence of ART. On Day 55, EGFR⁺ CAR T cells were detected in the peripheral blood and in the bone marrow. As anticipated, these CAR T cells were mostly CD8⁺ (FIG. 15A). Importantly, they still expressed the memory cell markers CD62L and CD27 (FIG. 15B and FIG. 15C). Thus, these results demonstrate CMV-HIV CAR T cells derived from an HIV^{pos} individual established persistent T cell memory in the marrow of HIV-infected mice. No significant difference in HIV viral load was observed in the peripheral blood (data not shown), possibly due to the limited number of infused CAR T cell and the high level of active HIV infection at the time of CAR T cell infusion.

The results here showed the ability of the CMV/HIV-CAR T cells to control viremia in lymphoid tissues and it might help to eradicate reservoirs of persistent infection and latency, which is not possible with ART or other current antiviral approaches.

### Example 6: CMV-HIV CAR T Cells Exhibit Anti-HIV Activity in a Human HIV Patients

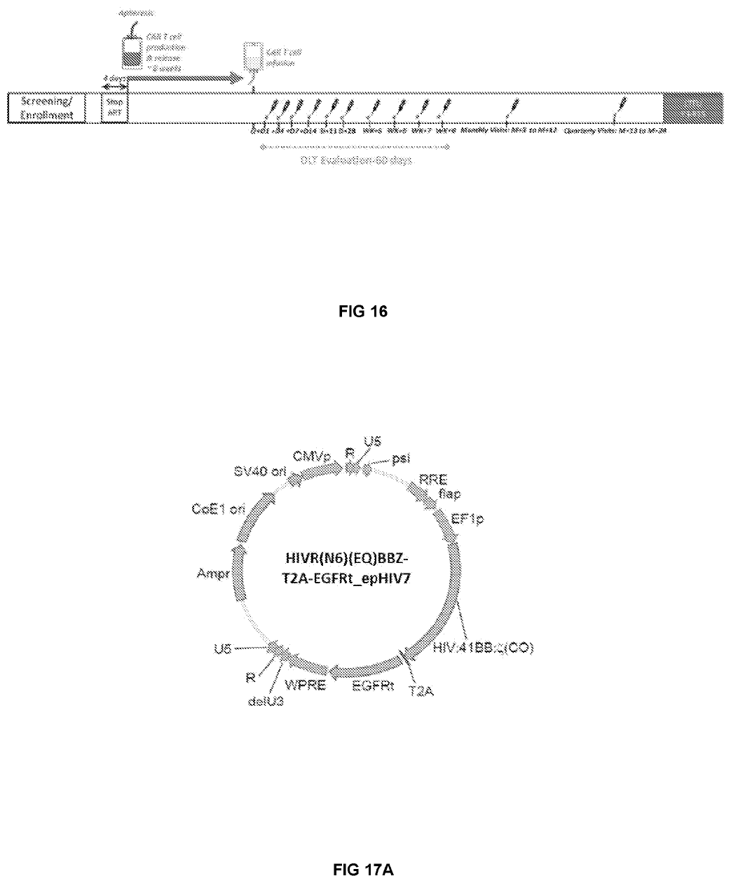

FIG. 16 depicts a schematic of the first in-human, single arm pilot study using autologous CMV/HIV-CAR T cells in PLWH who are stable virologically suppressed on long-term ART. The trial is designed to first study the safety of a single dose of CMV/HIV-CAR T cells at three dose levels. Each research participant will not be treated until the prior treated research participant has been monitored closely for a minimum of 60 days. Dose escalation, de-escalation, or expansion will not take place until at least three evaluable participants have accrued to the current dose level.

Step 1 of the protocol consists of screening and signing of the informed consent at UCSD and ACTG clinics. At step 2 of study entry, eligible participants temporarily interrupt their ART regimen for 4 days prior to leukapheresis to prevent inhibition of lentiviral transduction of the T cells during CAR T cell manufacturing. Participants will resume their ART regimen immediately after leukapheresis. During this 4-day ATI period, subjects will restart their prior ART regimen if any of the following occurs: (1) if requested by the participant or their HIV health-care provider, or (2) if ART is deemed medically necessary for non-HIV related causes, or (3) for symptomatic HIV disease (acute viral syndrome). If the manufacturing is not successful, a second apheresis may be scheduled no sooner than 3 weeks later, again with a 4-day ARV treatment interruption.

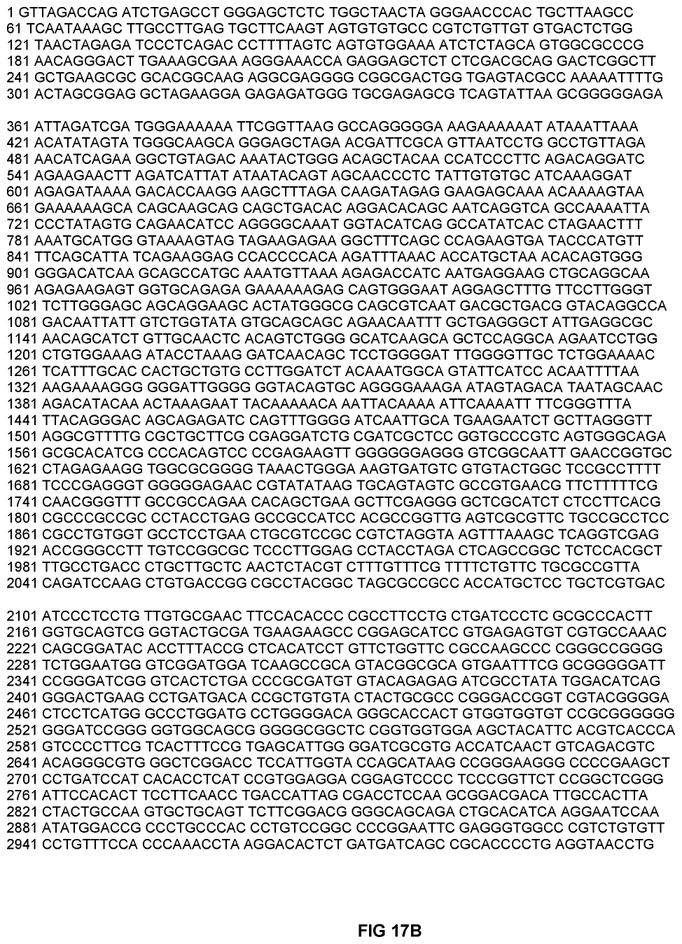

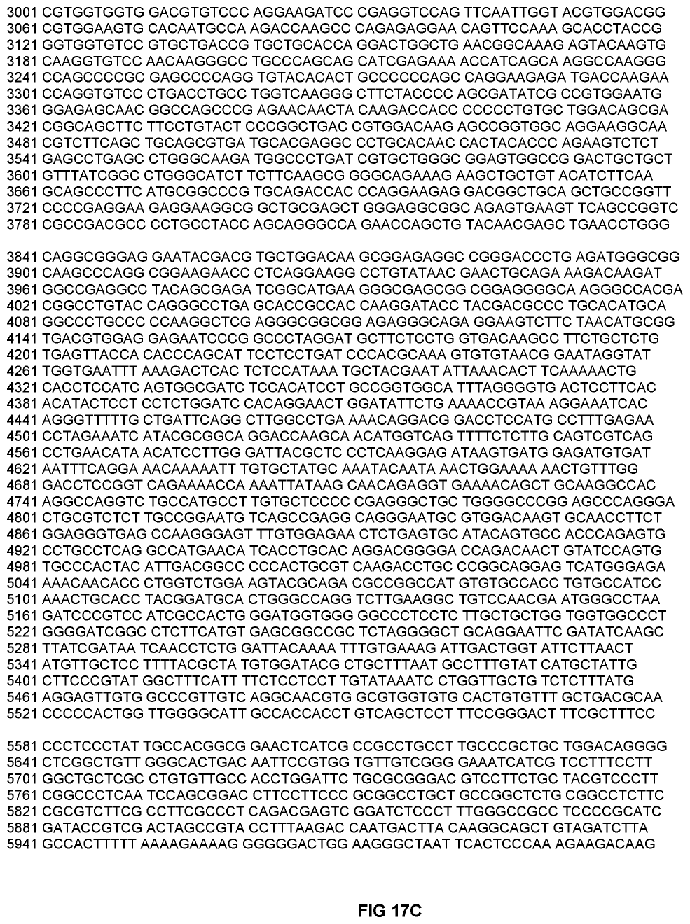

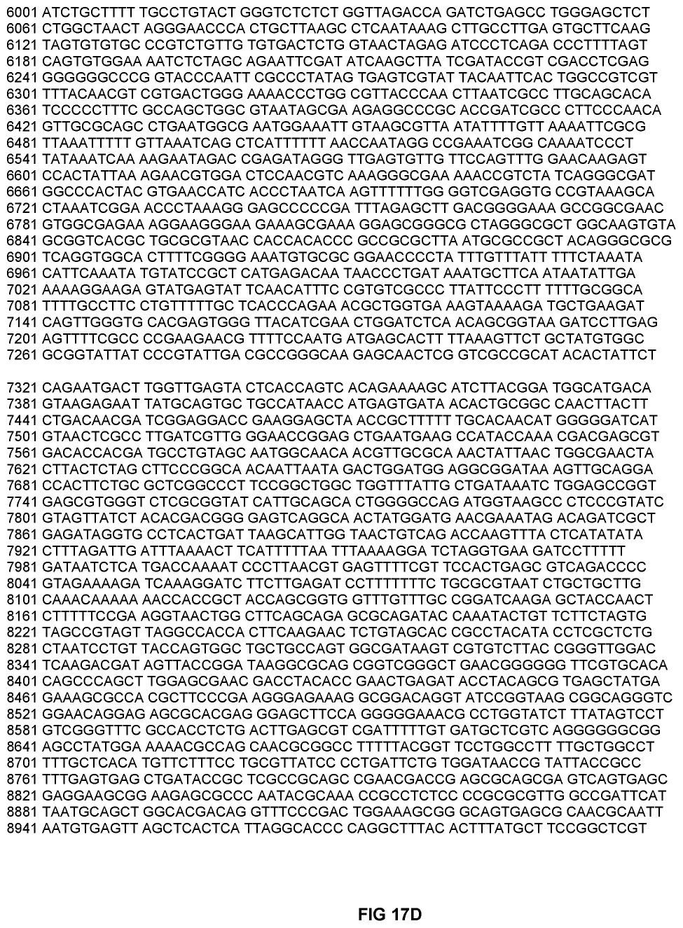

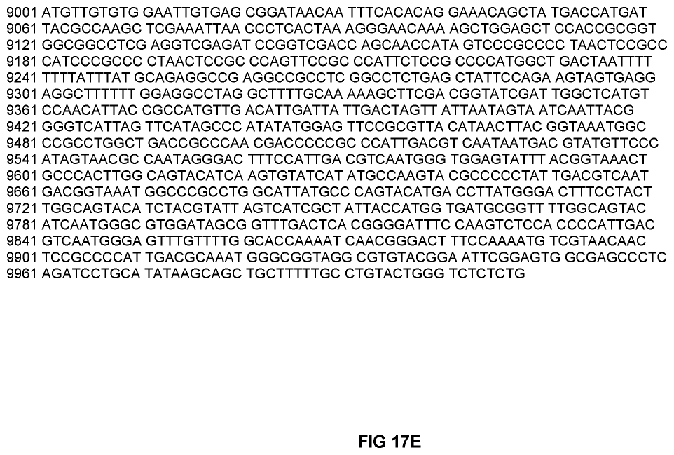

Research participants undergo a leukapheresis to collect peripheral blood mononuclear cells (PBMCs). The apheresis product is incubated overnight with CMV peptides, and CMV-specific T cells are enriched based on interferon gamma (IFNγ) positivity using the CliniMACS Prodigy® System (Miltenyi Biotec). These cells are then transduced with a self-inactivating lentiviral vector (vHIVR(N6)(EQ)BBK-T2A-EGFRt_epHIV7; FIGS. 17A-E) that directs the co-expression of gp120BBC-CAR (to target HIV gp120-expressing cells using scFv of the anti-gp120 bNAb N6) and truncated human epidermal growth factor receptor (EGFRt, used as a tracking marker). The resulting autologous CMV/HIV-CAR T cell product, i.e. the investigational agent, is expanded in vitro in presence of IL-2, IL-15, and ART cocktail inhibitor for ˜2 weeks and cryopreserved (FIG. 6A).

Once the final cell product is released, participants enter step 3. Participants assigned to dose level 1 receive a single intravenous (IV) infusion of 25×10⁶ cells autologous CMV/HIV-CAR T cells. Participants assigned to dose level 2 receive 50×10⁸ cells autologous CMV/HIV-CAR T cells. DLT evaluation period of the study is defined as Day −1 prior to CMV-HIV CAR T infusion through 60 days post CAR T-cell infusion.

A dose limiting toxicity (DLT) is defined as events considered at least possibly related to the CMV-HIV CAR T infusion with the exception of those listed in the below expected AEs (adverse events) and occurring within DLT evaluation period, unless otherwise specified. DLTs include:
Any grade 3 or higher organ toxicity (cardiac, dermatologic, gastrointestinal, hepatic, pulmonary, genitourinary, neurologic, hematologic, renal, secondary malignancy, and endocrine) designated as possibly, definitely, or probably related (level of attribution) to the infusion of the CAR T cells;
Any Grade 3 or greater cytokine release syndrome with an attribution of possible, probable or definite to CAR T-cell infusion;
Any Grade 3 or greater allergic reaction with an attribution of possible, probable or definite to CAR T-cell infusion;
Any Grade 3 or greater autoimmune toxicity with an attribution of possible, probable or definite to CAR T-cell infusion; or
Any Grade 5 toxicity with an attribution of possibly, probably or definitely related to the infusion of the CAR T cells.

Research participants may experience a number of “expected” AEs associated with the infusion of genetically modified T cells (usually occurring within the first 48 hours), and the in vivo expansion as well as the CAR-directed therapy (usually occurring within the first 21 days after CAR T cell infusion). The following is a list of highest allowable* “expected” AEs (including grade and duration) graded by CTCAE v5.0 excepting CRS/Neurotoxicity grading by ASTCT Consensus Criteria):
Dyspnea: Grade 3 dyspnea lasting up to 24 hours with intervention;
Fever: Grade 4 fever lasting up to 72 hours;
Cough: Grade 4 cough lasting up to 24 hours;
Headache: Grade 3 headache lasting up to 72 hours with intervention;
Hypotension: Hypotension: Grade 3 (without CRS symptoms) responding to fluid resuscitation and resolving to grade 2 or less within 24 hours;
Rash: Grade 3 rash lasting up to 72 hours with intervention.

Once safety of the process is shown in patients, a CMV vaccine (a CMV antigen or nucleic acid encoding a CMV antigen; e.g., CMV pp65) is added to the protocol depicted in FIG. 16. The CMV vaccine is administered to the patient prior to the CMV-HIV CAR T cell infusion (at day −1). This facilitates in vivo expansion of the CMV-HIV CAR T cells and increases the persistence of the memory T cells expressing memory cell markers CD62L and CD27. Some patients also receive a CMV vaccine or a CMV vaccine booster following the CAR T cell infusion (e.g., at day 1, 7, 10, 14, 21, 27, 30, 45, 60, 75, 90, 120, 150, 180, 210, 240, 270, 300, 330, and/or 360).

The CMV/HIV-CAR T cells control viremia in lymphoid tissues and eradicate reservoirs of persistent infection and latency. The single infusion of CAR T cells is designed to replace a lifetime regimen of ART (and other current antiviral approaches to treat HIV).

### Materials and Methods

The following materials and methods were used in the Examples.

### DNA Constructs

The N6-CAR construct was modified from the previously described CD19-specific scFvFc:ζ chimeric immunoreceptor.³ The HIV:41BB:ζ/EGFRt-epHIV7 lentiviral vector contains the GM-CSF receptor-α chain signal sequence (GMCSFRss), which enhances CAR surface expression, the CAR sequence consisting of the V_{H} and V_{L} gene segments of the N6 bNAb, the IgG4 hinge with two site mutations (L235E; N297Q) within the CH₂ region, the CD4 transmembrane and 4-1BB co-stimulatory domains, the cytoplasmic domain of the CD3ζ chain²⁴, the ribosomal skip T2A sequence, and the truncated human EGFR (EGFRt) sequence as previously described to allow for CAR T cell enrichment, tracking and potential cell ablation through ADCC.⁶⁴ The full CAR sequence is available upon request. The lentiviral vector encoding eGFP and ffLuc was created by removing the STOP codon in the eGFP open reading frame from pFUGW (Addgene plasmid #14883) and inserting a P2A-ffLuc-STOP cassette in frame with eGFP.

### Clinical Scale Production of CMV-HIV CAR T Cells

Fresh blood products were obtained from CMV^{pos} HIV^{neg} donors (StemCell Technologies, Vancouver, Canada) and HIV^{pos} donors on ART (Zen-Bio Inc, Research Triangle Park, NC, see Table 1). All procedures were performed in accordance with the Declaration of Helsinki Protocols (KC15TISI0494). CMV-specific T cells were isolated on the CliniMACS Prodigy and cytokine capture system (CCS) (Miltenyi Biotec) according to the manufacturer's instruction. Briefly, PBMCs were isolated and purified by density gradient centrifugation over Ficoll-Paque (Pharmacia Biotech, Sweden). After PBMCs (10⁹) were added to the application bag connected to the tubing set, the CliniMACS Prodigy device automatically performed successive processes, including sample washing, antigen stimulation with PepTivator CMVpp65, Catchmatrix labeling, anti-IFN-γ microbead labeling, magnetic enrichment and elution. CMV-specific cells and non-CMV-specific cells were eluted in separate bags following magnetic enrichment. After overnight rest in RPMI medium containing 10% human AB serum (Gemini Bio Products, Sacramento, CA), IL-2 (50 U/mL) and IL-15 (1 ng/mL), recovered IFN-γ⁺ cells (˜1×10⁶) were transduced at MOI 3 with the research (for HIV^{neg} donors and HIV^{pos} donors #551 and #552) or GMP-grade (for HIV^{pos} donors #553, #572, #573, IEQR #2, IEQR #3) lentiviral vector HIV:41BBζ:EGFRt-epHIV7. Fresh culture medium and cytokines were added every other day for ˜15 days. Antiretroviral drugs (43 nM darunavir and 279 nM enfuvirtide) were added twice per week during the expansion of HIV^{pos} #573, IEQR #2 and IEQR #3 derived CMV-HIV CAR T cells. Cultures were maintained at 37° C. under 5% (v/v) CO₂.

### Cell Lines

8E5 cells contain a single defective proviral genome of HIV and therefore are not infectious but express most of the HIV viral proteins including gp120. 8E5 (CRL-8993) cells were purchased from ATCC and maintained in RPMI 1640 (Irvine Scientific) medium supplemented with 10% heat-inactivated FCS (Hyclone). To generate 8E5 cell lines expressing enhanced green fluorescent protein (eGFP) and firefly luciferase (ffLuc), 8E5 cells were transduced with a lentiviral vector encoding eGFP-ffLuc, and GFP⁺gp120⁺ cells were sorted and expanded. Acute myeloid leukemia (AML) cell line KG-1a (CCL-246.1) cells were purchased from ATCC and maintained in 10% FCS IMDM medium and used as negative target cells. LCL-OKT3 cells were generated as previously described and served as positive T cell stimulator.²³ Cells were grown in complete medium supplemented with 0.4 mg/mL hygromycin. eGFP⁺ Jurkat cells were infected with the HIV_{NL4-3} and maintained in culture for 2 weeks. HEK293-eGFP-ffLuc-gp160 cells used as positive control cell lines for the tissue cross reactivity study were obtained by stably transfecting HEK-293T cell lines with C97ZA012 gp160 construct to express the cell surface gp160 (eventually cleaved into gp120 and gp41 HIV-1 envelope proteins). Banks of all cell lines were authenticated for the desired antigen/marker expression by flow cytometry prior to cryopreservation, and thawed cells were cultured for less than 3 months prior to use in assays.

### Cytotoxicity Assay

CAR T cell products (2.5×10⁵) and eGFP-positive target cells (8E5-gp120, 8E5, LCL-OKT3 or KG-1a) were cocultured at various effector-to-target (E:T) ratios of total T cells:target (2:1, 1:1, 1:2 or 1:5) for 4 days. Cocultures with LCL-OKT3, and 8E5 or KG-1a were used as positive and negative target controls. The cells were stained with anti-CD3 antibody. The percentages of viable eGFP⁺ CD3⁻ tumor cells were measured using multicolor flow cytometry. The “% of cytotoxicity” was calculated as following: 100%−(% of remaining tumor cells in CAR T cell group/% of remaining tumor cells in mock T or negative target groups). eGFP⁺ HIV_{N4-3}-infected Jurkat cells (2.5×10⁵) were cocultured with CMV-HIV CAR T cells at various E:T ratios (1:1, 1:2, 1:4) for 7 days. Cells then were stained with Viability Dye eFluor 450 (Miltenyi) for flow cytometric analysis of viable eGFP⁺ cells.

### Proliferation Assay

CAR T cell products (2.5×10⁵) were labeled with 0.5 μM CellTrace™ Violet dye (CTV) and cocultured with 8,000 cGy-irradiated stimulator cells LCL-OKT3, 8E5-gp120 and KG-1a, or autologous CMVpp65-peptide pulsed PBMCs which had been 3,500 cGy-irradiated at 1:1 ratio for 8 days. Cocultures with LCL-OKT3, KG-1a cells and media were used as positive and negative controls. Proliferation of CD3⁺ and EGFR⁺ populations was determined using multicolor flow cytometry.

### Intracellular IFN-γ Staining

CAR T cell products (10⁵) were activated overnight with LCL-OKT3, 8E5-gp120, or KG-1a cells (10⁵) in 96-well tissue culture plates, or with CMVpp65 peptide-pulsed autologous PBMC cells (10⁵) in the presence of Brefeldin A (BD Biosciences, Franklin Lakes, NJ). The cell mixture was then stained with anti-CD8 antibody, anti-EGFR antibody cetuximab, and streptavidin to analyze surface expression of CD8 and CAR, respectively. Cells were then fixed and permeabilized using the BD Cytofix/Cytoperm kit (BD Biosciences). After fixation, the T cells were stained with an anti-IFN-γ antibody. Cells were then analyzed using multicolor flow cytometry on MACSQuant (Miltenyi Biotec Inc.).

### Mice

Studies were performed with male and female NOD.Cg-Prkdc^{scid Il2rgtm1WjI/}SzJ (NSG) mice (JAX stock #005557) aged 3-5 weeks at the initiation of studies. Mice were group-housed in individually ventilated cages (OptiCages, Animal Care Systems) on com-cob bedding (‘Bed-o’-Cobs ⅛ in., The Andersons, Maumee, OH) with a square nestlet and PVC tube provided for enrichment. Mice were allowed free access to rodent chow (LabDiet 5350) and autoclaved acidified reverse osmosis purified water (pH 2.4 to 2.8) in bottles. After inoculation with HIV, mice were housed under animal biosafety level-2 (ABSL-2) conditions, group-housed in static disposable cages (Innocage, Innovive). The room temperature was held at a range of 68 to 79° F. and the room humidity range was 30% to 70%. Mice were designated as specific-pathogen-free (SPF) for mouse rotavirus, Sendai virus, pneumonia virus of mice, mouse hepatitis virus, minute virus of mice, mice parvovirus, Theiler murine encephalomyelitis virus, mouse reovirus type 3, mouse norovirus, lymphocytic choriomeningitis virus, mouse thymic virus, mouse adenovirus types 1 and 2, mouse cytomegalovirus, polyomavirus, K virus, ectromelia virus, Hantavirus, Prospect Hill virus, Filobacterium rodentium, Encephalitozoon cuniculi, and Mycoplasma pulmonis, Helicobacter spp., Clostridium piliforme, and free of any endo- and ectoparasites. Mice were maintained in accordance with the Guide for the Care and Use of Laboratory Animals in a facility accredited by the American Association for the Accreditation of Laboratory Animal Care (AAALAC). All experiments were performed according to the guidelines of the Institutional Animal Committee of the Beckman Research Institute of the City of Hope, IACUC 16095.

### Engraftment of Hu-PBMC-NSG Mice and HIV Challenge

PBMCs were collected from an HIV^{neg} or HIV^{pos} donor to manufacture CMV-HIV CAR T cells. The CMV-negative fraction of autologous PBMCs was cryopreserved as control T cells. CMV-negative PBMCs (1×10⁶) were mixed with CMV-negative resting PBMCs (9×10⁶) prior to transplantation on Day 0 in each mouse. Cells (1×10⁷ per mouse) were resuspended in sterile saline and injected intraperitoneally (IP) into NSG mice. Before treatment, mice were randomized to assure similar engraftment and gender proportions across groups. On Day 7, mice were challenged with HIV-1 BaL via IP injection. Longitudinal blood collections were performed using retro-orbital bleeding on anesthetized mice and peripheral blood cell populations and plasma viral loads were analyzed periodically using flow cytometry and qRT-PCR. Mice that did not engraft huCD45⁺ cells (defined as >30 cells/μL huCD45⁺ cells in peripheral blood) were excluded for analysis. Mice showing severe signs of GHVD were immediately humanely euthanized.

### Oral ART Therapy

Infected mice with detectable viral infection (defined as >10³ cp/mL of HIV in blood) were treated orally for 3 weeks with ART composed of drugs that block new infections, without inhibiting viral production in infected cells. The ART regimen consisting of Truvada® [tenofovir disoproxil fumarate (TDF; 300 mg/tablet), emtricitabine (FTC; 200 mg/tablet) (Gilead Sciences)] and Isentress® [raltegravir (RAL; 400 mg/tablet) (Merck)], scaled down to the equivalent mouse dosage using the appropriate conversion factor, was administered in a drinking water formulation (sweetened water gel, Medidrop® Sucralose, ClearH20). For 400 mL Medidrop®, ½ Truvada tablet and ½ Isentress tablet were crushed to powder and mixed by shaking bottle to a homogenous solution; medicated water was changed weekly. Doses of ART drugs were calculated based on previous studies using the same delivery system.⁶⁵

### CMV-HIV CAR T Cells and In Vivo CMVpp65 Stimulation

Mice received CMV-HIV CAR T cells (0.05 to 1×10⁶ EGFR+ T cells), CMV-negative T cells (1×10⁶); or autologous PBMCs as control T cells by retro-orbital injection under general isoflurane anesthesia. Autologous PBMCs were pulsed with CMVpp65 peptide mix (#PM-PP65, JPT Peptide Technologies, Germany) as antigen presenting cells (APCs) as “CMVpp65 vaccine”. When indicated, mice received CMVpp65 peptide-pulsed and irradiated (3500 rads) autologous CMV-negative PBMCs (5×10⁶) by retro-orbital injection under general isoflurane anesthesia.

### Antibodies

Fluorochrome-conjugated isotype controls against CD3 (#563109), CD4 (#557582), CD8 (#348793), IFN-γ (#554701), CD27 (#555440), CD45RA (#550855), CD62L (#341012), CD127 (#560822), programmed cell death-1 (PD-1) (#551892), lymphocyte-activation gene-3 (LAG-3, #565720) and T cell immunoglobulin and mucin domain-3 (Tim-3, #563422) were obtained from BD Biosciences (San Diego, CA). The following reagent was obtained through the NIH AIDS Reagent Program, Division of AIDS, NIAID, NIH: anti-HIV-1 gp120 Monoclonal (VRC01) from Dr. John Mascola (cat #12033). Biotinylated anti-EGFR antibody Erbitux® (cetuximab) was obtained from the City of Hope pharmacy. Antibody against EGFR was obtained from eBioscience (San Diego, CA). CellTrace™ Violet dye (CTV) was purchased from Invitrogen (Carlsbad, CA). All monoclonal antibodies and CTV were used according to the manufacturer's instructions.

### Reagents

CliniMACS Prodigy® TS500 tubing sets, MACS GMP PepTivator® HCMV pp65, CCS Reagent, CliniMACS PBS/EDTA buffer and TexMACSM GMP medium were all purchased from Miltenyi Biotec. CliniMACS PBS/EDTA with 2.5% human serum albumin (HSA; Grifols Therapeutics, Los Angeles, CA) was used as the elution buffer. GMP-grade cell transfer bags and luer/spike adaptors were purchased from BD Medical (Franklin Lakes, NJ). pepMix HCMVA (pp65; pp65pepmix) was purchased from JPT Peptide Technologies. GmbH was used for pulsation on PBMCs according to the manufacturer's instructions. Antiretroviral drug darunavir was obtained through the NIH HIV Reagent Program, Division of AIDS, NIAID, NIH (Cat #11447) from Tibotec, Inc and enfuvirtide (Fuzeon, Genentech) were reconstituted in water.

Synthesis of scFv-Fc of N6

The anti-gp120 N6 monoclonal antibody (mAb) variable domains were reformatted into a recombinant single-chain scFv-Fc antibody fragment. The cDNA encoding the N6 variable light and heavy chain domains (in VL-linker-VH-orientation) were synthesized with a (Gly4Ser)3 linker and fused to an IgG4 Fc domain. Briefly, the scFv-Fc of N6 was cloned into the Lonza pEE12.4 vector and transiently transfected using the EXP1293 expression system. The culture was then clarified by centrifugation (1,000×g, 5 min), followed by 0.22 μm sterile filtration. The clarified harvest was treated overnight with AG 1-X8 strong anion exchange resin and affinity purified by protein A chromatography (ProSep vA high-capacity resin, EMD Millipore). Pooled eluates containing N6 scFv-Fc (V_{L}-V_{H}) were dialyzed using a Slide-A-Lyzer 20 k MWCO cassette vs. PBS buffer. The final dialyzed sample was sterile filtered using 0.22 μm PES filter membrane and stored at 4° C. The test reagent was assayed for expression by SDS-PAGE and ELISA assays.

### Tissue Cross-Reactivity Analysis

Charles River Laboratories, Inc. performed the cross-reactivity study of N6 scFv-Fc. First, N6 scFv-Fc was tested for specific reaction on positive control (gp160-transfected HEK293T cells expressing gp120) and negative control parental HEK293T cells (gp120-negative) at 5 μg/mL and 15 μg/mL. The test article was substituted with a human IgG4K antibody, designated HuIgG4 (control article) and other controls were produced by omission of the test or control articles from the assay (assay control). The tissue panel used as the test system for the in vitro cross-reactivity study includes all the tissues recommended in the FDA, Center for Biologics Evaluation and Research (CBER) document Points to Consider in the Manufacture and Testing of Monoclonal Antibody Products for Human Use. Fresh unfixed tissues were collected as surgical or autopsy specimens from humans and frozen in Tissue-Tek® OCT at −85-70° C. Sections were cut at approximately 5 μm and fixed in acetone for 10 min at room temperature. Just prior to staining, the slides were fixed in 10% neutral-buffered formalin (NBF) for 10 seconds at room temperature. The labeled secondary antibody was allowed to attach specifically to the unlabeled primary antibody (either test or control article at 5 μg/mL and 15 μg/mL) by overnight incubation of the primary/secondary antibody mixtures. The test or control article was mixed with biotinylated F(ab′)2 donkey anti-human IgG, Fcγ fragment-specific (DkαHuIgG) antibody at concentrations which achieved a primary:secondary antibody ratio of 1:1.5. Precomplexed antibodies were incubated overnight at 2 to 8° C. Prior to use of the antibody on the subsequent day, human gamma globulins were added to each vial to achieve a final concentration of either 4.5 mg/mL (higher concentration of secondary antibody) or 1.5 mg/mL (lower concentration of secondary antibody), and antibodies were incubated for at least 2 hours at 2 to 8° C. On the day of staining, the slides were rinsed twice with Tris-buffered saline, 0.15M NaCl, pH 7.6 (TBS). Next, the slides were incubated with the avidin solution for 15 min, rinsed once with TBS, incubated with the biotin solution for 15 min, and rinsed once with TBS. The slides were then treated for 20 min with a protein block (TBS+1% bovine serum albumin (BSA); 0.5% casein; and 1.5% normal donkey serum) designed to reduce nonspecific binding. Following the protein block, the precomplexed primary and secondary antibodies were applied to the slides for 2 hours. Next, the slides were rinsed twice with TBS, and endogenous peroxidase was then quenched by incubation of the slides with the Dako peroxidase blocking reagent for 5 min. Next, the slides were rinsed twice with TBS, treated with the ABC Elite reagent for 30 min, rinsed twice with TBS, and then treated with DAB for 4 min as a substrate for the peroxidase reaction. All slides were rinsed with tap water, counterstained, dehydrated, and mounted. TBS+1% BSA served as the diluent for all antibodies and ABC reagent. Separate cryosections from each human test tissue were stained in parallel for the expression of human β2-microglobulin (a relatively ubiquitous epitope) using a polyclonal rabbit antibody directed against human β2-microglobulin. All evaluated human test tissues stained positive for β2-microglobulin, indicating their suitability in the cross-reactivity evaluation. After staining, slides were visualized and evaluated under light microscopy by a pathologist.

### Flow Cytometry

Cells were stained with optimized antibody panels for 20 min at 4° C. followed by two washes with PBS. Data acquisition for all experiments involving flow cytometry was performed on a MACSquant (Miltenyi Biotec) and analyzed using FCS Express V7 (De Novo Software, Glendale, CA).

Peripheral blood samples were collected by retro-orbital bleeding under general anesthesia and stained for 30 min with BV711-conjugated antihuman CD3, APC-conjugated antihuman CD4, BB515-conjugated antihuman CD8, BUV395-conjugated antihuman CD45 (BD Biosciences, San Jose, CA), and BV421-conjugated antihuman EGFR (Biolegend, San Diego, CA). Stained peripheral blood samples were then lysed with red blood cell lysis buffer and absolute cell counts calculated using BD Liquid Counting Beads (BD Biosciences, San Jose, CA). Flow cytometry was performed using BD Fortessa II instrument (BD Biosciences) and analyzed with FlowJo software (BD formerly TreeStar).

Tissue samples were collected at necropsy and processed immediately for cell isolation and flow cytometry analysis. Bone marrow mononuclear cell suspensions were first stained with amine binding dye for dead cell exclusion (Biolegend) and then stained with anti-human-CD3 (BD clone UCHT1), -EGFR (Miltenyi biotinylated clone REA688), -CD4 (Biolegend clone RPA-T4), anti-human CD8 (BD clone RPA-T8), -CD62L (Biolegend clone DREG-56), and -CD27 (Biolegend clone M-T271) in brilliant staining buffer (BD) containing 0.5% human serum albumen and 0.5% gamma globulin. Primary EGFR antibody staining was finished with a streptavidin conjugate (eBioscience) and fixed in 4% PFA. Samples were acquired the next day on a BD Fortessa SORP cytometer. Data was analyzed using FlowJo Software (BD formerly TreeStar). Cell doublets and dead cells were excluded prior to evaluation of the T cell lineage and phenotypic markers.

### Intracellular HIV p24 Staining

Samples of peripheral blood and single cell suspensions of mouse bone marrow (femurs+/−tibias) were collected at time of euthanasia. Single cell suspensions were made following previously established protocols.¹ Briefly for bone marrow cells, femurs and tibia were dissected and collected from euthanized mice and placed in ice cold PBS. Bones were cleaned thoroughly to remove all connective and muscle tissue, then using a scalpel blade the heads of the bones were removed. Bones were placed in 0.5 mL microcentrifuge tube with a premade hole by using a 20 g needle. Bones were placed cut surface down and a 0.5 mL tube was placed in a 1.5 mL microcentrifuge tube and centrifuged at >10,000×g for 15 sec. Cell pellet was resuspended in ACK lysis buffer incubated for 5 min and washed with PBS. Cells were resuspended in PBS+2% FBS and then processed for FACS staining or frozen in 10% Cryostor (Stem Cell Technologies, Vancouver, BC). For intracellular staining, BD Cytofix/Cytoperm™ kit (BD Biosciences, San Jose, CA) was used following manufacture's protocol. After surface markers staining (CD45, CD3, CD4, CD8, EGFR), cells were permeabilized and intracellular staining of KC57-FITC monoclonal antibody Fortessa II instrument (BD Biosciences) and analyzed with FlowJo software (BD formerly TreeStar).

### Elisa Assay

Quantification of HIV-1 p24 was measured on the supernatants as per the manufacturer's instructions (Alliance ELISA; Perkin-Elmer Life Sciences, Boston, MA) with the assay's Lower Limit of Quantification (LLOQ) being 12.5 pg/mL.

Plasma HIV qRT-PCR

Plasma viremia was assayed using one-step reverse transcriptase real-time PCR (TaqMan assay] with automated CFX96 Touch™ Real Time PCR Detection System (Bio-Rad). qPCR primer sets were taken from previously published studies.² HIV-1 level in peripheral blood was determined by extracting RNA from blood plasma using the QIAamp Viral RNA mini kit (Qiagen) and performing Taqman qPCR using either a primer and probe set targeting the HIV-1 LTR region [FPrimer GCCTCAATAAAGCTTGCCTTGA, RPrimer: GGCGCCACTGCTAGAGATTTT, Probe: 5′FAM/AAGTAGTGTGTGCCCGTCTGTTGTGTGACT/3IABkFQ] or the HIV-1 Pol region [FPrimer: GACTGTAGTCCAGGAATATG, RPrimer: TGTTTCCTGCCC TGTCTC, Probe: 5′Cy5/CTTGGTAGCAGTTCATGTAGCCAG/3′IABkFQ], using the TaqMan Fast Virus 1-Step Master Mix (Applied Biosystems). According to the manufacturer's instruction (QIAamp Viral RNA mini kit [Qiagen]), the protocol is designed for purification of viral RNA from minimal 140 μL plasma. In a standard Taqman qPCR-based HIV-1 plasm viral load test, the limit of detection (LOD) is typically about 40 copies/mL when viral RNA isolated from 140 μL of plasma sample is applied. In our animal study, the plasma sample was expanded by dilution (generally 1 to 3 dilution) because only limited volume of plasma (20-40 μL) was available. The LOD of the diluted samples was around ˜2,000 RNA copies/mL using the HIV LTR primer and ˜500 RNA copies/mL using the HIV Pol primer under our experimental condition. Therefore, we defined that the value below those LOD numbers is undetectable.

### Statistical Analysis

Analyses were performed using Prism (GraphPad Software Inc.) or R version 4.0.266 and are described in the individual figure legends. A significance level of 0.05 was used for all analyses.
1. Gunthard, H. F., Saag. M. S., Benson, C. A., del Rio, C., Eron, J. J., Gallant, J. E., Hoy, J. F., Mugavero, M. J., Sax, P. E., Thompson, M. A., Gandhi, R. T., et al. (2016). Antiretroviral Drugs for Treatment and Prevention of HIV Infection in Adults: 2016 Recommendations of the International Antiviral Society—USA Panel. Jama 316, 191-210. 10.1001/jama.2016.8900.
2. Sadowski, I., and Hashemi, F. B. (2019). Strategies to eradicate HIV from infected patients: elimination of latent provirus reservoirs. Cellular and molecular life sciences: CMLS 76, 3583-3600. 10.1007/s00018-019-03156-8.
3. Hege, K. M., Cooke, K. S., Finer, M. H., Zsebo, K. M., and Roberts, M. R. (1996). Systemic T cell-independent tumor immunity after transplantation of universal receptor-modified bone marrow into SCID mice. The Journal of experimental medicine 184, 2261-2269. 10.1084/jem.184.6.2261.
4. Wagner, T. A. (2018). Quarter Century of Anti-HIV CAR T Cells. Current HIV/AIDS reports 15, 147-154. 10.1007/s11904-018-0388-x.
5. Patel, S., Hanajiri, R., Grant, M., Saunders, D., Van Pelt, S., Keller, M., Hanley, P. J., Simon, G., Nixon, D. F., Hardy, D., Jones, R. B., et al. (2020). HIV-Specific T Cells Can Be Generated against Non-escaped T Cell Epitopes with a GMP-Compliant Manufacturing Platform. Molecular therapy. Methods & clinical development 16, 11-20. 10.1016/j.omtm.2019.10.001.
6. Romeo, C., and Seed, B. (1991). Cellular immunity to HIV activated by CD4 fused to T cell or Fc receptor polypeptides. Cell 64, 1037-1046.
7. Roberts, M. R., Qin, L., Zhang, D., Smith, D. H., Tran, A., Duil, T. J., Groopman, J. E., Capon, D. J., Bym, R. A., and Finer, M. H. (1994). Targeting of Human Immunodeficiency Virus-Infected Cells by CD8+T Lymphocytes Armed With Universal T-Cells Receptors. Blood 84, 2878-2889.
8. Mitsuyasu, R. T., Anton, P. A., Deeks, S. G., Scadden, D. T., Connick, E., Downs, M. T., Bakker, A., Roberts, M. R., June, C. H., Jalali, S., Lin, A. A., et al. (2000). Prolonged survival and tissue trafficking following adoptive transfer of CD4zeta gene-modified autologous CD4(+) and CD8(+) T cells in human immunodeficiency virus-infected subjects. Blood 96, 785-793.
9. Deeks, S. G., Wagner, B., Anton, P. A., Mitsuyasu, R. T., Scadden, D. T., Huang, C., Macken, C., Richman, D. D., Christopherson, C., June, C. H., Lazar, R., et al. (2002). A phase II randomized study of HIV-specific T-cell gene therapy in subjects with undetectable plasma viremia on combination antiretroviral therapy. Molecular therapy: the journal of the American Society of Gene Therapy 5, 788-797.
10 Walker, R. E., Bechtel, C. M., Natarajan, V., Baseler, M., Hege, K. M., Metcalf, J. A., Stevens. R., Hazen, A., Blaese, R. M., Chen, C. C., Leitman, S. F., et al. (2000). Long-term in vivo survival of receptor-modified syngeneic T cells in patients with human immunodeficiency virus infection. Blood 96, 487-474.
11 Scholler, J., Brady, T. L., Binder-Scholl. G., Hwang, W. T., Plesa, G., Hege, K. M., Vogel, A. N., Kalos, M., Riley, J. L., Deeks, S. G., Mitsuyasu, R. T., et al. (2012). Decade-long safety and function of retroviral-modified chimeric antigen receptor T cells. Science translational medicine 4, 132ra153. 10.1126/scitranslmed.3003761.
12 van der Stegen, S. J., Hamieh, M., and Sadelain, M. (2015). The pharmacology of second-generation chimeric antigen receptors. Nat Rev Drug Discov 14, 499-509. 10.1038/nrd4597.
13 Kuhlmann, A. S., Peterson, C. W., and Kiem, H. P. (2018). Chimeric antigen receptor T-cell approaches to HIV cure. Current opinion in HIV and AIDS. 10.1097/COH.0000000000000485.
14 Ying, Z., He, T., Wang, X., Zheng, W., Lin, N., Tu, M., Xie, Y., Ping, L., Zhang, C., Liu, W., Deng, L., et al. (2019). Parallel Comparison of 4-1BB or CD28 Co-stimulated CD19-Targeted CAR-T Cells for B Cell Non-Hodgkin's Lymphoma. Molecular therapy oncolytics 15, 60-68. 10.1016/j.omto.2019.08.002.
15 Zhong, Q., Zhu, Y. M., Zheng, L. L., Shen, H. J., Ou, R. M., Liu, Z., She, Y. L., Chen, R., Li, C., Huang, J., Yao, M. D., et al. (2018). Chimeric Antigen Receptor-T Cells with 4-1BB Co-Stimulatory Domain Present a Superior Treatment Outcome than Those with CD28 Domain Based on Bioinformatics. Ada haematologica 140, 131-140. 10.1159/000492146.
16 Hale, M., Mesojednik, T., Romano Ibarra, G. S., Sahni, J., Bernard, A., Sommer, K., Scharenberg, A. M., Rawlings, D. J., and Wagner, T. A. (2017). Engineering HIV-Resistant, Anti-HIV Chimeric Antigen Receptor T Cells. Molecular therapy: the journal of the American Society of Gene Therapy 25, 570-579. 10.1016/j.ymthe.2016.12.023.
17 Mu, W., Carrillo, M. A., and Kitchen, S. G. (2020). Engineering CAR T Cells to Target the HIV Reservoir. Frontiers in cellular and infection microbiology 10, 410. 10.3389/fcimb.2020.00410.
18. Huang, J., Kang, B. H., Ishida, E., Zhou, T., Griesman, T., Sheng, Z., Wu, F., Doria-Rose, N. A., Zhang, B., McKee, K., O'Dell, S., et al. (2016). Identification of a CD4-Binding-Site Antibody to HIV that Evolved Near-Pan Neutralization Breadth. Immunity 45, 1108-1121. 10.1016/j.immuni.2016.10.027.
19. Lapteva, N., Gilbert, M., Diaconu, I., Rollins, L. A., Al-Sabbagh, M., Naik, S., Krance, R. A., Tripic, T., Hiregange, M., Raghavan, D., Dakhova, O., et al. (2019). T-Cell Receptor Stimulation Enhances the Expansion and Function of CD19 Chimeric Antigen Receptor-Expressing T Cells. Clin Cancer Res 25, 7340-7350. 10.1158/1078-0432.ccr-18-3199.
20. Pule, M. A., Savoldo, B., Myers, G. D., Rossig, C., Russell, H. V., Dotti, G., Huls, M. H., Liu, E., Gee, A. P., Mei, Z., Yvon, E., et al. (2008). Virus-specific T cells engineered to coexpress tumor-specific receptors: persistence and antitumor activity in individuals with neuroblastoma. Nat Med 14, 1264-1270. 10.1038/nm.1882.
21. Savoldo, B., Rooney, C. M., Di Stasi, A., Abken, H., Hombach, A., Foster, A. E., Zhang, L., Heslop, H. E., Brenner, M. K., and Dotti, G. (2007). Epstein Barr virus specific cytotoxic T lymphocytes expressing the anti-CD30zeta artificial chimeric T-cell receptor for immunotherapy of Hodgkin disease. Blood 110, 2620-2630. 10.1182/blood-2006-11-059139.
22. Cooper, L. J., Al-Kadhimi, Z., Serrano, L. M., Pfeiffer, T., Olivares, S., Castro, A., Chang, W. C., Gonzalez, S., Smith, D., Forman, S. J., and Jensen, M. C. (2005). Enhanced antilymphoma efficacy of CD19-redirected influenza MP1-specific CTLs by cotransfer of T cells modified to present influenza MP1. Blood 105, 1622-1631. 10.1182/blood-2004-03-1208.
23. Wang, X., Wong, C. W., Urak, R., Mardiros, A., Budde, L. E., Chang, W. C., Thomas, S. H., Brown, C. E., La Rosa, C., Diamond, D. J., Jensen, M. C., et al. (2015). CMVpp65 Vaccine Enhances the Antitumor Efficacy of Adoptively Transferred CD19-Redirected CMV-Specific T Cells. Clinical cancer research: an official journal of the American Association for Cancer Research. 10.1158/1078-0432.ccr-14-2920.
24. Jonnalagadda, M., Mardiros, A., Urak, R., Wang, X., Hoffman, L. J., Bemanke, A., Chang, W. C., Bretzlaff, W., Starr, R., Priceman, S., Ostberg, J. R., et al. (2015). Chimeric antigen receptors with mutated IgG4 Fc spacer avoid fc receptor binding and improve T cell persistence and antitumor efficacy. Molecular therapy: the journal of the American Society of Gene Therapy 23, 757-768. 10.1038/mt.2014.208.
25. Philipson, B. I., O'Connor, R. S., May, M. J., June, C. H., Albelda, S. M., and Milone, M. C. (2020). 4-1BB costimulation promotes CAR T cell survival through noncanonical NF-κB signaling. Science signaling 13. 10.1126/scisignal.aay8248.
26. Frigault, M. J., Lee, J., Basil, M. C., Carpenito, C., Motohashi, S., Scholler, J., Kawalekar, O. U., Guedan, S., McGettigan. S. E., Posey. A. D., Jr., Ang, S., et al. (2015). Identification of chimeric antigen receptors that mediate constitutive or inducible proliferation of T cells. Cancer immunology research 3, 356-367. 10.1158/2326-6066.CIR-14-0186.
27. Brentjens, R. J., Riviere, I., Park, J. H., Davila, M. L., Wang, X., Stefanski, J., Taylor, C., Yeh, R., Bartido, S., Borquez-Ojeda, O., Olszewska, M., et al. (2011). Safety and persistence of adoptively transferred autologous CD19-targeted T cells in patients with relapsed or chemotherapy refractory B-cell leukemias. Blood 118, 4817-4828. 10.1182/blood-2011-04-348540.
28. Porter, D. L., Hwang, W. T., Frey, N. V., Lacey, S. F., Shaw, P. A., Loren, A. W., Bagg, A., Marcucci, K. T., Shen, A., Gonzalez, V., Ambrose, D., et al. (2015). Chimeric antigen receptor T cells persist and induce sustained remissions in relapsed refractory chronic lymphocytic leukemia. Science translational medicine 7, 303ra139. 10.1126/scitranslmed.aac5415.
29. Leibman, R. S., Richardson, M. W., Ellebrecht, C. T., Maldini, C. R., Glover, J. A., Secreto, A. J., Kulikovskaya, I., Lacey, S. F., Akkina, S. R., Yi, Y., Shaheen, F., et al. (2017). Supraphysiologic control over HIV-1 replication mediated by CD8 T cells expressing a re-engineered CD4-based chimeric antigen receptor. PLoS pathogens 13, e1006613. 10.1371/journal.ppat.1006613.
30. Wang, X., Chang, W. C., Wong, C. W., Colcher, D., Sherman, M., Ostberg, J. R., Forman, S. J., Riddell, S. R., and Jensen, M. C. (2011). A transgene-encoded cell surface polypeptide for selection, in vivo tracking, and ablation of engineered cells. Blood 118, 1255-1263. 10.1182/blood-2011-02-337360.
31. Hall, W. C., Price-Schiavi, S. A., Wicks, J., and Rojko, J. L. (2008). Tissue Cross-Reactivity Studies for Monoclonal Antibodies: Predictive Value and Use for Selection of Relevant Animal Species for Toxicity Testing.
32. Leach, M. W., Halpem, W. G., Johnson, C. W., Rojko, J. L., MacLachlan, T. K., Chan, C. M., Galbreath, E. J., Ndifor, A. M., Blanset, D. L., Polack, E., and Cavagnaro, J. A. (2010). Use of tissue cross-reactivity studies in the development of antibody-based biopharmaceuticals: history, experience, methodology, and future directions. Toxicologic pathology 38, 1138-1166. 10.1177/0192623310382559.
33. Kumaresan, P., Figliola, M., Moyes, J. S., Huls, M. H., Tewari, P., Shpall, E. J., Champlin, R., and Cooper, L. J. (2015). Automated Cell Enrichment of Cytomegalovirus-specific T cells for Clinical Applications using the Cytokine-capture System. Journal of visualized experiments: JoVE. 10.3791/52808.
34. Wang, X., Urak, R., Walter, M., Guan, M., Han, T., Vyas, V., Chien, S. H., Gittins, B., Clark, M. C., Mokhtari, S., Cardoso, A., et al. (2022). Large-scale manufacturing and characterization of CMV-CD19CAR T cells. Journal for immunotherapy of cancer 10. 10.1136/jitc-2021-003461.
35. Naeger, D. M., Martin, J. N., Sinclair, E., Hunt, P. W., Bangsberg, D. R., Hecht, F., Hsue, P., McCune, J. M., and Deeks, S. G. (2010). Cytomegalovirus-specific T cells persist at very high levels during long-term antiretroviral treatment of HIV disease. PloS one 5, e8886. 10.1371/journal.pone.0008886.
38. Stone, S. F., Price, P., Khan, N., Moss, P. A., and French, M. A. (2005). HIV patients on antiretroviral therapy have high frequencies of CD8 T cells specific for Immediate Early protein-1 of cytomegalovirus. AIDS 19, 555-562.
37. Okhrimenko, A., Grun, J. R., Westendorf, K., Fang, Z., Reinke, S., von Roth, P., Wassilew, G., Kuhl, A. A., Kudematsch, R., Demski, S., Scheibenbogen, C., et al. (2014). Human memory T cells from the bone marrow are resting and maintain long-lasting systemic memory. Proceedings of the National Academy of Sciences of the United States of America 111, 9229-9234. 10.1073/pnas.1318731111.
38. Walker, B., and McMichael, A. (2012). The T-cell response to HIV. Cold Spring Harbor perspectives in medicine 2. 10.1101/cshperspect.a007054.
39. Okulicz, J. F., and Lambotte, O. (2011). Epidemiology and clinical characteristics of elite controllers. Current opinion in HIV and AIDS 6, 163-168. 10.1097/COH.0b013e32834f35e.
40. Loffredo, J. T., Sidney, J., Bean, A. T., Beal, D. R., Bardet, W., Wahl, A., Hawkins, O. E., Piaskowski, S., Wilson, N. A., Hildebrand, W. H., Watkins, D. I., et al. (2009). Two MHC class I molecules associated with elite control of immunodeficiency virus replication, Mamu-B*08 and HLA-B*2705, bind peptides with sequence similarity. Journal of immunology (Baltimore, Md.: 1950) 182, 7763-7775. 10.4049/jimmunol.0900111.
41. Saez-Cirion, A., Lacabaratz, C., Lambotte, O., Versmisse. P., Urrutia, A., Boufassa, F., Barre-Sinoussi, F., Delfraissy. J. F., Sinet, M., Pancino, G., and Venet, A. (2007). HIV controllers exhibit potent CD8 T cell capacity to suppress HIV infection ex vivo and peculiar cytotoxic T lymphocyte activation phenotype. Proceedings of the National Academy of Sciences of the United States of America 104, 6776-6781.
42. Chomont, N., El-Far, M., Ancuta, P., Trautmann. L., Procopio, F. A., Yassine-Diab. B., Boucher, G., Boulassel, M. R., Ghattas, G., Brenchley, J. M., Schacker, T. W., et al. (2009). HIV reservoir size and persistence are driven by T cell survival and homeostatic proliferation. Nature Medicine 15, 893-900. nm.1972 [pii]
43. Jiang, C., Lian, X., Gao, C., Sun, X., Einkauf, K. B., Chevalier, J. M., Chen, S. M. Y., Hua, S., Rhee, B., Chang, K., Blackmer, J. E., et al. (2020). Distinct viral reservoirs in individuals with spontaneous control of HIV-1. Nature 585, 261-267. 10.1038/s41586-020-2651-8.
44. Chomont, N. (2020). HIV enters deep sleep in people who naturally control the virus. Nature 585, 190-191. 10.1038/d41586-020-02438-7.
45. Hua, C. K., and Ackerman, M. E. (2016). Engineering broadly neutralizing antibodies for HIV prevention and therapy. Advanced drug delivery reviews 103, 157-173. 10.1016fj.addr.2016.01.013.
46. Rust, B. J., Kean, L. S., Colonna, L., Brandenstein. K. E., Poole, N. H., Obenza, W., Enstrom, M. R., Maldini, C. R., Ellis, G. I., Fennessey, C. M., Huang. M. L., et al. (2020). Robust expansion of HIV CAR T cells following antigen boosting in ART-suppressed nonhuman primates. Blood 136, 1722-1734. 10.1182/blood.2020006372.
47. Hebeis, B. J., Klenovsek, K., Rohwer, P., Ritter, U., Schneider, A., Mach, M., and Winkler, T. H. (2004). Activation of virus-specific memory B cells in the absence of T cell help. The Journal of experimental medicine 199, 593-602. 10.1084fjem.20030091.
48. Wang, X., Wong, C. W., Urak, R., Mardiros, A., Budde, L. E., Chang, W. C., Thomas, S. H., Brown, C. E., La Rosa, C., Diamond, D. J., Jensen, M. C., et al. (2015). CMVpp65 Vaccine Enhances the Antitumor Efficacy of Adoptively Transferred CD19-Redirected CMV-Specific T Cells. Clinical cancer research: an official journal of the American Association for Cancer Research 21, 2993-3002. 10.1158/1078-0432.Ccr-14-2920.
49. Holguin, L., Echavarria, L., and Burnett, J. C. (2022). Novel Humanized Peripheral Blood Mononuclear Cell Mouse Model with Delayed Onset of Graft-versus-Host Disease for Preclinical HIV Research. J Virol 96, e0139421. 10.1128/jvi.01394-21.
50. Pampusch, M. S., Abdelaal, H. M., Cartwright, E. K., Molden, J. S., Davey, B. C., Sauve, J. D., Sevcik, E. N., Rendahl, A. K., Rakasz, E. G., Connick, E., Berger, E. A., et al. (2022). CAR/CXCR5-T cell immunotherapy is safe and potentially efficacious in promoting sustained remission of SIV infection. PLoS pathogens 18, e1009831. 10.1371/journal.ppat.1009831.
51. Zhen, A., Carrillo, M. A., Mu, W., Rezek, V., Martin, H., Hamid, P., Chen, I. S. Y., Yang, O. O., Zack, J. A., and Kitchen, S. G. (2021). Robust CAR-T memory formation and function via hematopoietic stem cell delivery. PLoS pathogens 17, e1009404. 10.1371/journal.ppat.1009404.
52. Maldini, C. R., Gayout, K., Leibman, R. S., Dopkin, D. L., Mills, J. P., Shan, X., Glover, J. A., and Riley, J. L. (2020). HIV-Resistant and HIV-Specific CAR-Modified CD4(+) T Cells Mitigate HIV Disease Progression and Confer CD4(+) T Cell Help In Vivo. Molecular therapy: the journal of the American Society of Gene Therapy 28, 1585-1599. 10.1016/j.ymthe.2020.05.012.
53. Maldini, C. R., Claiborne, D. T., Okawa, K., Chen, T., Dopkin, D. L., Shan, X., Power, K. A., Trifonova, R. T., Krupp, K., Phelps, M., Vrbanac, V. D., et al. (2020). Dual CD4-based CAR T cells with distinct costimulatory domains mitigate HIV pathogenesis in vivo. Nat Med 26, 1776-1787. 10.1038/s41591-020-1039-5.
54. Julg, B., Pegu, A., Abbink, P., Liu, J., Brinkman, A., Molloy, K., Mojta, S., Chandrashekar, A., Callow, K., Wang, K., Chen, X., et al. (2017). Virological Control by the CD4-Binding Site Antibody N6 in Simian-Human Immunodeficiency Virus-Infected Rhesus Monkeys. J Virol 91. 10.1128/JVI.00498-17.
55. Anthony-Gonda, K., Bardhi, A., Ray, A., Flerin, N., Li, M., Chen, W., Ochsenbauer, C., Kappes, J. C., Krueger, W., Worden, A., Schneider, D., et al. (2019). Multispecific anti-HIV duoCAR-T cells display broad in vitro antiviral activity and potent in vivo elimination of HIV-infected cells in a humanized mouse model. Science translational medicine 11. 10.1126/scitransimed.aav5685.
56 de Larrea, C. F., Staehr, M., Lopez, A. V., Ng, K. Y., Chen, Y., Godfrey, W. D., Purdon, T. J., Ponomarev, V., Wendel. H. G., Brentjens, R. J., and Smith. E. L. (2020). Defining an Optimal Dual-Targeted CAR T-cell Therapy Approach Simultaneously Targeting BCMA and GPRCSD to Prevent BCMA Escape-Driven Relapse in Multiple Myeloma. Blood Cancer Discov 1, 146-154. 10.1158/2643-3230.bcd-20-0020.
57. Qin, H., Ramakrishna, S., Nguyen, S., Fountaine, T. J., Ponduri, A., Stetler-Stevenson, M., Yuan, C. M., Haso, W., Shem, J. F., Shah, N. N., and Fry, T. J. (2018). Preclinical Development of Bivalent Chimeric Antigen Receptors Targeting Both CD19 and CD22. Mol Ther Oncolytics 11, 127-137. 10.1016/j.omto.2018.10.006.
58. Kang, L., Zhang, J., Li. M., Xu, N., Qi, W., Tan, J., Lou, X., Yu, Z., Sun, J., Wang, Z., Fu, C., et al. (2020). Characterization of novel dual tandem CD19/BCMA chimeric antigen receptor T cells to potentially treat multiple myeloma. Biomark Res 8, 14. 10.1186/s40364-020-00192-8.
59. Zah, E., Nam, E., Bhuvan, V., Tran, U., Ji, B. Y., Gosliner, S. B., Wang, X., Brown, C. E., and Chen, Y. Y. (2020). Systematically optimized BCMA/CS1 bispecific CAR-T cells robustly control heterogeneous multiple myeloma. Nat Commun 11, 2283. 10.1038/s41467-020-16160-5.
60. La Rosa, C., Longmate, J., Martinez, J., Zhou, Q., Kaltcheva, T. I., Tsai, W., Drake, J., Carroll, M., Wussow, F., Chiuppesi, F., Hardwick, N., et al. (2017). MVA vaccine encoding CMV antigens safely induces durable expansion of CMV-specific T-cells in healthy adults. Blood. 10.1182/blood-2016-07-729756.
61. Aldoss, I., La Rosa, C., Baden, L. R., Longmate, J., Ariza-Heredia, E. J., Rida, W. N., Lingaraju, C. R., Zhou, Q., Martinez, J., Kaltcheva, T., Dagis, A., et al. (2020). Poxvirus Vectored Cytomegalovirus Vaccine to Prevent Cytomegalovirus Viremia in Transplant Recipients: A Phase 2, Randomized Clinical Trial. Ann Intern Med 172, 306-316. 10.7326/M19-2511.
62. Diamond, C. S. G. W. a. D. (A5355) Phase II, Double-Blind, Randomized, Placebo-Controlled Trial to Evaluate the Safety and Immunogenicity of a Modified Vaccinia Ankara (MVA)-based anti-Cytomegalovirus (CMV) Vaccine (Triplex®), in Adults with Both Human Immunodeficiency Virus (HIV)-1 and CMV Who Are on Potent Combination ART with Conserved Immune Function. NIAID.
63. Kowolik, C. M., Topp, M. S., Gonzalez, S., Pfeiffer, T., Olivares, S., Gonzalez, N., Smith, D. D., Forman, S. J., Jensen, M. C., and Cooper, L. J. (2006). CD28 costimulation provided through a CD19-specific chimeric antigen receptor enhances in vivo persistence and antitumor efficacy of adoptively transferred T cells. Cancer Res 66, 10995-11004.
64. Wang, X., Li, H., Matte-Martone, C., Cui, W., Li, N., Tan, H. S., Roopenian, D., and Shlomchik, W. D. (2011). Mechanisms of antigen presentation to T cells in murine graft-versus-host disease: cross-presentation and the appearance of cross-presentation. Blood 118, 6426-6437. 10.1182/blood-2011-06-358747.
65. Satheesan, S., Li, H., Burnett, J. C., Takahashi, M., Li, S., Wu, S. X., Synold, T. W., Rossi, J. J., and Zhou, J. (2018). HIV Replication and Latency in a Humanized NSG Mouse Model during Suppressive Oral Combinational Antiretroviral Therapy. J Virol 92. 10.1128/JVI.02118-17.
66 R Core Team, R.F.f.S.C. (2020). R: A Language and Environment for Statistical Computing.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A nucleic acid molecule encoding a chimeric antigen receptor, wherein the chimeric antigen receptor comprises:
(i) an scFv that binds HIV Env;
(ii) a spacer domain;
(iii) a transmembrane domain;
(iv) a costimulatory domain; and
(v) a CD3ζ signaling domain.

2. The nucleic acid molecule of claim 1, wherein the scFv comprises: a VL domain comprising: a light chain CDR1 comprising QTSQGVGSDLH, a light chain CDR2 comprising HTSSVED, a light chain CDR3 comprising QVLQF; and a VH domain comprising: a heavy chain CDR1 comprising AHILF, a heavy chain CDR2 comprising WIKPQYGAVNFGGGFRD, and a heavy chain CDR3 comprising DRSYGDSSWALDA.

3. The nucleic acid molecule of claim 1, wherein the scFV comprises:
(a) a light chain variable domain that is at least 90%, 95% or 98% identical to:
YIHVTQSPSSLSVSIGDRVTINCQTSQGVGSDLHWYQHKPGRAPKLLIH
HTSSVEDGVPSRFSGSGFHTSFNLTISDLQADDIATYYCQVLQFFGRGS
RLHIK;
(b) a heavy chain variable domain that is at least 90%, 95% or 98% identical to:
RAHLVQSGTAMKKPGASVRVSCQTSGYTFTAHILFWFRQAPGRGLEWVG
WIKPQYGAVNFGGGFRDRVTLTRDVYREIAYMDIRGLKPDDTAVYYCAR
DRSYGDSSWALDAWGQGTTVVVSA

4. The nucleic acid molecule of claim 3, wherein the scFV comprises: a light chain variable domain comprising
YIHVTQSPSSLSVSIGDRVTINCQTSQGVGSDLHWYQHKPGRAPKLLIH
HTSSVEDGVPSRFSGSGFHTSFNLTISDLQADDIATYYCQVLQFFGRGS
RLHIK;
a heavy chain variable domain comprising
RAHLVQSGTAMKKPGASVRVSCQTSGYTFTAHILFWFRQAPGRGLEWVG
WIKPQYGAVNFGGGFRDRVTLTRDVYREIAYMDIRGLKPDDTAVYYCAR
DRSYGDSSWALDAWGQGTTVVVS.

5. The nucleic acid molecule of claim 3, wherein the scFv comprises a VL domain that is 95% identical to YIHVTQSPSSLSVSIGDRVTINCQTSQGVGSDLHWYQHKPGRAPKLLIHHTSSVEDGVPSR FSGSGFHTSFNLTISDLQADDIATYYCQVLQFFGRGSRLHIK and includes the following CDR sequences: QTSQGVGSDLH (VL-CDR1), HTSSVED (VL-CDR2), and QVLQF (VL-CDR3); and a VH domain that is 95% identical to RAHLVQSGTAMKKPGASVRVSCQTSGYTFTAHILFWFRQAPGRGLEWVGWIKPQYGAVN FGGGFRDRVTLTRDVYREIAYMDIRGLKPDDTAVYYCARDRSYGDSSWALDAWGQGTTVV VSA and includes the following CDR sequences AHILF (VH-CDR1) WIKPQYGAVNFGGGFRD (VH-CDR2), and DRSYGDSSWALDA (VH-CDR3).

6. A nucleic acid molecule encoding a chimeric antigen receptor, wherein the chimeric antigen receptor comprises: a scFv comprising or consisting of RAHLVQSGTAMKKPGASVRVSCQTSGYTFTAHILFWFRQAPGRGLEWVGWIKPQYGAVN FGGGFRDRVTLTRDVYREIAYMDIRGLKPDDTAVYYCARDRSYGDSSWALDAWGQGTTVV VSAGGGSGGGSGGGSGGGSYIHVTQSPSSLSVSIGDRVTINCQTSQGVGSDLHWYQHKP GRAPKLLIHHTSSVEDGVPSRFSGSGFHTSFNLTISDLQADDIATYYCQVLQFFGRGSRLHI K; a spacer comprising a sequence selected from the group consisting of: SEQ ID NOs: 24-34; a transmembrane domain comprising a sequence selected from the group consisting of SEQ ID NOs: 15-23; a costimulatory domain comprising a sequence selected from the group consisting of SEQ ID NOs: 36-40, and a CD3ζ signaling domain comprising SEQ ID NO: 35.

7. The nucleic acid molecule of any of claims 1-6, wherein the spacer region comprises an amino acid sequence selected from the group consisting of SEQ ID NOs: 24-34.

8. The nucleic acid molecule of any of claims 1-6, wherein the transmembrane domain selected from the group consisting of:
a CD4 transmembrane domain,
a CD8 transmembrane domain,
a CD28 transmembrane domain, and
a CD3ζ transmembrane domain;

9. The nucleic acid molecule of any of claims 1-6, wherein the costimulatory domain selected from the group consisting of
a CD28 costimulatory domain,
a 41-BB costimulatory domain,
an OX40 costimulatory domain, and
a 2B4 costimulatory domain.

10. The nucleic acid molecule of claim 1, wherein the chimeric antigen receptor comprises the amino acid sequence RAHLVQSGTAMKKPGASVRVSCQTSGYTFTAHILFWFRQAPGRGLEWVGWIKPQYGAVN FGGGFRDRVTLTRDVYREIAYMDIRGLKPDDTAVYYCARDRSYGDSSWALDAWGQGTTW VSAGGGSGGGSGGGSGGGSYIHVTQSPSSLSVSIGDRVTINCQTSQGVGSDLHWYQHKP GRAPKLLIHHTSSVEDGVPSRFSGSGFHTSFNLTISDLQADDIATYYCQVLQFFGRGSRLHI KESKYGPPCPPCPAPEFEGGPSVFLFPPKPKDTLMISRTPEVTCVVDVSQEDPEVQFNW YVDGVEVHNAKTKPREEQFQSTYRWSVLTVLHQDWLNGKEYKCKVSNKGLPSSIEKTISK AKGQPREPQVYTLPPSQEEMTKNQVSLTCLVKGFYPSDIAVEWESNGQPENNYKTTPPVL DSDGSFFLYSRLTVDKSRWQEGNVFSCSVMHEALHNHYTQKSLSLSLGKMALIVLGGVAG LLLFIGLGIFFKRGRKKLLYIFKQPFMRPVQTTQEEDGCSCRFPEEEEGGCELGGGRVKFSR SADAPAYQQGQNQLYNELNLGRREEYDVLDKRRGRDPEMGGKPRRKNPQEGLYNELQK DKMAEAYSEIGMKGERRRGKGHDGLYQGLSTATKDTYDALHMQALPPR with 0, 1, 2, 3, 4 of 5 single amino acid substitutions.

11. The nucleic acid molecule of any of claims 1-6, further comprising an interdomain linker consisting of 1-5 amino acids between one or more of: the scFV and the spacer domain, the spacer domain and the transmembrane domain, the transmembrane domain and the co-stimulatory domain, and the costimulatory domain and the CD3ζ signaling domain.

12. The nucleic acid molecule of claim 11, wherein interdomain linker consists of 1-5 glycine.

13. The nucleic acid molecule of any of claims 1-6, wherein an interdomain linker consisting of the sequence GGG is located between the costimulatory domain and the CD3ζ signaling domain.

14. An immune cell harboring the nucleic acid molecule of any of the forgoing claims.

15. The immune cell of claim 14, wherein the cell is a T cell expressing a T cell receptor specific for CMV (CMV specific T cell).

16. A population of cells comprising CMV specific T cells harboring the nucleic acid molecule of any of claims 1-13.

17. The population of cells of claim 16, wherein at least 50% of the CMV specific T cells are CD8+ T cells.

18. A method of preparing the population of CMV specific T cells expressing an HIV CAR, the method comprising: isolating a cell population comprising PBMC from a blood sample obtained from a CMV^{pos} subject; contacting the cell population with a CMV antigen to stimulate CMV specific T cells; isolating a sub-population of IFNγ-secreting T cells (e.g., CMV specific cells) from the cell population; and transducing cells in the sub-population of IFNγ-secreting T cells with a vector comprising the nucleic acid molecule of any of claims 1-14.

19. The method of claim 18, wherein the sub-population of IFNγ-secreting T cells are cultured in the presence of one or both of exogenous IL-2 and exogenous IL-15 before transduction, after transduction or both before and after transduction.

20. The method of claim 19, wherein IL-2 is added to at 50 U/mL and IL-15 is added to 1 ng/mL.

21. The method of claim 19, wherein the sub-population of IFNγ-secreting T cells are cultured in the presence of at least one or both of an HIV protease inhibitor and an HIV entry/fusion inhibitor and not in the presence of a reverse transcriptase inhibitor before transduction, after transduction or both before and after transduction.

22. The method of claim 21, wherein the sub-population of IFNγ-secreting T cells are cultured in the presence of darunavir and enfuvirtide.

23. The method of any of claims 18-19, wherein the blood sample is from a subject infected with HIV.

24. A method for treating a subject infected with HIV, the method comprising administering: (a) a population of CMV-specific T cells expressing chimeric antigen receptor comprising: an scFv that binds HIV Env; a spacer domain; a transmembrane domain; a costimulatory domain; and a CD3ζ signaling domain; and, optionally, (b) at least one CMV antigen or a nucleic acid molecule encoding at least one CMV antigen.

25. The method of claim 24, wherein the at least one CMV antigen comprises at least one of a CMV protein, a fragment of a CMV protein, a modified CMV protein, a fragment of a modified CMV protein, a mutated CMV protein or a fragment thereof, a fusion CMV protein or a fragment thereof, and combinations thereof.

26. The method of claim 24, wherein the at least one CMV antigen comprises at least one of pp65, IE1 exon 4 (IE1/e4), IE2 exon 5 (IE2/e5), fusions thereof, antigenic fragments thereof, and variants thereof having 1, 2, 3, 4, or 5 amino acid modifications.

27. The method of claim 24, wherein the at least one CMV antigen comprises a CMVpp65 peptide.

28. The method of claim 24, wherein the nucleic acid molecule comprises a viral vector encoding (a) a CMV pp65 peptide or protein and (b) a fusion protein comprising exon 4 of CMV protein 1E1 (e4) and exon 5 of CMV protein 1E2 (e5).

29. The method of claim 24, wherein the at least one antigen comprises at least one sequence selected from SEQ ID NOs: 57-64 and variants thereof having 1, 2, 3, 4, or 5 amino acid modifications.

30. The method of claim 24, wherein the at least one CMV antigen comprises at least one fragment of any of SEQ ID NOs: 57-64, wherein the fragment comprises of consists of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 contiguous amino acids of any of SEQ ID NOs: 57-64.

31. The method of claim 24, wherein the at least one CMV antigen or a nucleic acid molecule encoding at least one CMV antigen is administered prior to or subsequent to administering the population of CMV-specific T cells.

32. The method of claim 31, wherein the at least one CMV antigen or a nucleic acid molecule encoding at least one CMV antigen is administered about 1, 2, 3, 4, 5, 6, 7, 8, 9, 10, 11, 12, 14, 16, 18, 20, 21, 22, 23, 24, 25, 26, 28, 36, 48, 60, 75, 90, 120, 150, 180, 210, 240, 270, 300, 330, and/or 360 hours prior to administering the population of CMV-specific T cells.

33. The method of claim 31, wherein the at least one CMV antigen or a nucleic acid molecule encoding at least one CMV antigen is administered about 1, 2, 3, 4, 5, 6, 7, 8, 9, 10, 11, 12, 14, 16, 18, 20, 21, 22, 23, 24, 25, 26, 28, 36, 48, 60, 75, 90, 120, 150, 180, 210, 240, 270, 300, 330, and/or 360 days subsequent to administering the population of CMV-specific T cells.

34. The method of claim 24, wherein the at least one CMV antigen or a nucleic acid molecule encoding at least one CMV antigen is administered in an amount sufficient to illicit an immune response in the subject.

35. The method of claim 24, wherein the CAR comprises: a scFv comprising or consisting of SEQ ID NO:9 or a variant thereof having 1, 2, 3, 4, or 5, amino acid substitutions that are not in a CDR; a spacer comprising a sequence selected from the group consisting of: SEQ ID NOs: 24-34 or a variant thereof having 1, 2, 3, 4, or 5, amino acid substitutions; a transmembrane domain comprising a sequence selected from the group consisting of SEQ ID NOs: 15-23 or a variant thereof having 1, 2, 3, 4, or 5, amino acid substitutions; a costimulatory domain comprising a sequence selected from the group consisting of SEQ ID NOs: 36-40 or a variant thereof having 1, 2, 3, 4, or 5, amino acid substitutions; and a CD3ζ signaling domain comprising SEQ ID NO: 35 or a variant thereof having 1, 2, 3, 4, or 5, amino acid substitutions.

36. The method of claim 24, wherein the CAR comprises SEQ ID NO:10 or a variant thereof having 1, 2, 3, 4, or 5, amino acid substitutions, wherein the amino acid substitutions are not in the CDRs.

37. The method of claim 24, wherein the population of CMV-specific T cells is at least 40%, 50%, 60%, or 70% IFN-γ positive.

38. The method of claim 24, wherein the population of CMV-specific T cells is at least 20%, 30%, 35% CD8 positive.

39. The method of claim 24, wherein the population of CMV-specific T cells is at least 20%, 25%, 30%, 35% CD4 positive.

40. The method of claim 24, wherein the population of CMV-specific T cells is at least 40% IFN-γ positive, at least 20% CD8 positive, and at least 20% CD4 positive.

41. The method of claim 24, wherein the population of CMV-specific T cells is at least 20% CD8 positive and at least 20% CD4 positive.

42. The method of claim 41, wherein the population of CMV-specific T cells is at least 30% CD8 positive and at least 30% CD4 positive.
